(19) United States
(12) Patent Application Publication
(10) Pub. No.: US 2025/0114350 A1
(43) Pub. Date: Apr. 10, 2025

(54) INHIBITORS OF PRENYLTRANSFERASES AS ANTIFUNGAL AGENTS

(71) Applicants: Regents of the University of Minnesota, Minneapolis, MN (US); Duke University, Durham, NC (US)

(72) Inventors: Mark Dewey Distefano, Minneapolis, MN (US); Feng Xu, Minneapolis, MN (US); Lorena S. Beese, Durham, NC (US); Homme W. Hellinga, Durham, NC (US); You Wang, Durham, NC (US); Andrew A. Alspaugh, Durham, NC (US)

(73) Assignees: Regents of the University of Minnesota, Minneapolis, MN (US); Duke University, Durham, NC (US)

(21) Appl. No.: 18/907,040

(22) Filed: Oct. 4, 2024

Related U.S. Application Data
(60) Provisional application No. 63/542,834, filed on Oct. 6, 2023.

(51) Int. Cl. A61K 31/496; A61P 31/10

(52) U.S. Cl. CPC A61K 31/496; A61P 31/10

(57) ABSTRACT

The invention provides competitive inhibitors of farnesyl transferase, compositions comprising the competitive inhibitors of farnesyl transferase, and their use as antifungal agents and as agents for the prevention of the formation or growth of biofilms.

### CROSS-REFERENCE TO RELATED APPLICATION

This application claims priority to U.S. Provisional Application No. 63/542,834 that was filed on Oct. 6, 2023. The entire content of the applications referenced above is hereby incorporated by reference herein.

### STATEMENT REGARDING FEDERALLY SPONSORED RESEARCH

This invention was made with government support under AI104533 and GM141853 awarded by the National Institutes of Health. The government has certain rights in the invention.

### BACKGROUND

Farnesyl transferase (FTase) catalyzes the sequence-specific covalent addition of a 15-carbon farnesyl moiety to a cysteine located within C-terminal tetra-peptide sequences in a protein (FIG. 1). Of particular interest is the farnesylation of Ras, which is required for its membrane localization, without which Ras does not function (Zhang, F. L, et al., Annual Review of Biochemistry, 1996, 65, 241-269; and Jiang, H.; Zhang, et al., Chemical reviews, 2018, 118, 919-988). Because Ras-mediated signaling controls a wide range of essential cellular activities in organisms as divergent as humans and fungi (Pentland, D. R, et al., Microbial Cell 2018, 5, 63-73), interference with its function can be a powerful therapeutic strategy. Genetic disruption of FTase in C. neoformans and other pathogenic fungi resulted in diminished stress tolerance and reduced virulence in animal models of infection (Esher, S. K, et al., mSphere, 2016, 1; Vallim, M. A, et al., Microbiology, 2004, 150, 1925-35; and Norton, T. S, et al., Virulence, 2017, 8, 1401-1416). Targeting FTase therefore is a promising strategy for antifungal discovery.

The structures of FTases of mammalian and fungal FTases bound to substrates and inhibitors have been studied extensively (Park, H. W, et al., Science, 1997, 275, 1800-4; Taylor, J. S, et al., EMBO J, 2003, 22, 5963-74; Long, S. B, et al., Biochemistry, 1998, 37, 9612-8; Long, S. B, et al., Structure, 2000, 8, 209-22; Long, S. B, et al., Nature, 2002, 419, 645-50; Strickland, C. L, et al., Biochemistry, 1998, 37, 16601-11; Mabanglo, M. F, et al., Protein Sci, 2014, 23, 289-301; Hast, M. A.; Beese, L. S., J Biol Chem, 2008, 283, 31933-40; Hast, M. A, et al., J Biol Chem, 2011, 286, 35149-62; and Reid, T. S, et al., Biochemistry, 2004, 43, 9000-8). The α/β FTase heterodimer is structurally highly conserved. The active site is positioned within the interface between the subunits (FIG. 1C). Farnesylation of the peptide substrate cysteine is catalyzed by a Zn²⁺ ion coordinated by three invariant residues in the β subunit. The peptide and farnesyl diphosphate (FPP) substrates bind adjacently within a deep groove at the interface. The prenylated peptide translocates to a second, orthogonal groove prior to release. The peptide-binding and exit groove surfaces are the only regions of significant structural divergence between FTase homologs.

Invasive fungal infections affect over a billion people worldwide, resulting in over 1.6 million deaths each year (Almeida, F., et al., Front Microbiol 2019, 10, 214). Of these, cryptococcosis, caused by members of the encapsulated yeast genus Cryptococcus, including C. neoformans, is associated with one million symptomatic infections and 181,000 deaths (Iyer, K. R, et al., Nature Reviews Microbiology, 2021, 19, 454-466). It is 100% lethal if left untreated. Most of these deaths occur in vulnerable populations with limited access to treatment or impaired immunity due to HIV infection, organ transplantation, or various types of malignancies (Schmiedel, Y.; Zimmerli, S., Swiss Med Wkly, 2016, 146, w14281). As with many fungal infections, the therapeutic arsenal to treat cryptococcosis is limited, and resistant strains are on the rise (Zafar, H, et al., Current Opinion in Microbiology, 2019, 52, 158-164; and Revie, N. M, et al., Current Opinion in Microbiology 45, 70-76). New antifungals to treat this and other fungal infections therefore are urgently needed.

### SUMMARY

In one aspect the present invention provides a method comprising, contacting a fungus (e.g., Cryptococcus neoformans) with an inhibitor of a fungal farnesyl transferase that is competitive with respect to a farnesyl disphosphate substrate.

In another aspect, the invention provides a method comprising, treating a fungal infection in a mammal by administering to the mammal an inhibitor of a fungal farnesyl transferase that is competitive with respect to farnesyl disphosphate substrate.

In another aspect, the invention provides a Compound 2f:

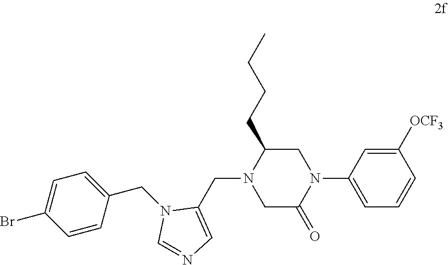 [SMILES: CCCC[C@H]1CN(c2cccc(OC(F)(F)F)c2)C(=O)CN1Cc1cncn1Cc1ccc(Br)cc1]

or a pharmaceutically acceptable salt thereof.

In another aspect, the invention provides a pharmaceutical composition comprising Compound 2f:

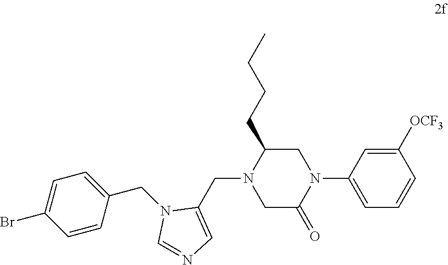 [SMILES: CCCC[C@H]1CN(c2cccc(OC(F)(F)F)c2)C(=O)CN1Cc1cncn1Cc1ccc(Br)cc1]

or a pharmaceutically acceptable salt thereof and a pharmaceutically acceptable excipient.

In another aspect, the invention provides a competitive inhibitor of a fungal farnesyl transferase for use in medical therapy.

In another aspect, the invention provides a competitive inhibitor of a fungal farnesyl transferase for the prophylactic or therapeutic treatment of a fungal infection.

In another aspect, the invention provides a composition comprising a competitive inhibitor of a fungal farnesyl transferase for promoting an antifungal effect.

In another aspect, the invention provides the use of a competitive inhibitor of a fungal farnesyl transferase to prepare a medicament for the treatment of a fungal infection in a mammal.

The invention also provides processes and intermediates disclosed herein that are useful for preparing competitive inhibitors of fungal farnesyl transferase or salts thereof.

### BRIEF DESCRIPTION OF THE DRAWINGS

FIG. 1. Structures. (A) The farnesyl diphosphate substrate. (B) The L-778,123 inhibitor comprises four flexibly connected rings (A-D), with five freely rotatable bonds. Rings A-C fold to form a compact core. The D ring is located on the surface of that core and free to rotate around χ₂₃. Within the core, the A ring can fold with two orientations, by rotation around χ₁₂. The locations of the R¹—R⁴ sites that were modified re indicated. (C) The CnFTase active site with bound FPP and L-778,123 (PDB 3Q7A). The B ring binds to the catalytic Zn²⁺ (magenta). The locations of the four rings and their modification sites relative to the peptide substrate binding-site and product exit groove are shown.

FIG. 2. The effects of the C325A mutation in the CnFTase β subunit on C. neoformans. The ram1Δ deletion strain was transformed with either wild-type RAM1 or mutant ram1-C325A. (A) Thermotolerance. Each strain was incubated in five-fold serial dilutions on solid YPD medium at 25° C., 30° C., and 37° C. Growth was assessed by inspection after 48 hours. (B) Mating. The two MATα strains (ram1Δ+RAM1 or ram1Δ+ram1-C325A) were incubated in mating co-cultures with an opposite mating type strain (KN99a-“MATa”) on MS mating medium. Mating competence was determined after 7 days by assessing the presence (wild-type, right) or absence (C325A mutant, left) of mating hyphae and sexual spore formation (inset).

FIG. 3. CnFTase is the in vivo target of 2f. (A) Ras cellular mislocalization, assessed by epifluorescent microscopy (1000×). Bar=10 μm. Top. In an untreated C. neoformans strain GFP-Ras1 localizes predominantly at the plasma membrane (left); in the presence of 0.78 μM 2f (sub-inhibitory), the fusion protein remains in the cytoplasm (right). Bottom. Control patterns of Ras localization with wild-type (left) and ram1Δ deletion (right) strains expressing mCherry-Ras1. (B) Overexpression of Ram1 negates 2f antifungal activity. A C. neoformans strain transformed with the galactose-regulated pGAL7-RAM1 over-expression construct was incubated with glucose (uninduced, bottom), and galactose (induced, top). Antifungal activity was assessed by the relative size of the zone of fungal growth inhibition surrounding sterile discs impregnated with 10 μL of 20 mM 2f dissolved in DMSO (bottom disk) or DMSO alone (top disc).

FIG. 4. The effects of phosphate on the steady-state kinetics of CnFTase. (A-C) FPP steady-binding. (D-F) 2f inhibition. (G-I) 2q inhibition. (A) Michaelis-Menten curves fit (2, 3) to the initial rate dependence on FPP concentration (1) determined at six phosphate concentrations (solid lines, fit; circles, observed initial rates). (D, G) Initial rates (1) measured at 1 uM FPP at six different phosphate concentrations (circles), fit (lines) to extract ^{1μM}IC₅₀ values (2, 3). (B, E, H) Extracted K_{M} and ^{1μM}IC₅₀ values were fit a two-state, single-site, isothermal phosphate-binding curves (2, 3) with phosphate dissociation constants varying 0.1-0.8 μM (lines, fit; circles, extracted values). For FFP (B) and 2f (E), the two states were modeled as constant α and β values in 2. For 2q (H) a linear post-transition baseline (4) was used for β in 2. (C, F, I) The extracted Vₘₐₓ values (C, circles) and inhibitor-free rates (F and I, circles) were fit (lines) to a two-state, single-site, isothermal phosphate-binding curve (2, 3) using linear post-transition baselines (4). The average mid-point of the effect of phosphate on affinities (B, E, H) is ˜0.5 mM. (J) A model for the effects of phosphate binding on enzyme activity and inhibition. The enzyme comprises two conformations, L and H, that are in equilibrium with each other. In the absence of phosphate, L (blue). The enzyme has a binding site for phosphate (P) at a location outside the active site (i.e. allosteric), with higher affinity for H than L. Consequently, phosphate binding shifts the equilibrium to H (green). The L and H conformations differ in catalytic efficiency (Vₘₐₓ), FPP affinity (K_{M}), and the ^{1μM}IC₅₀ values of 2f and 2q. (K) The occupancies of binding sites for FPP, (black) inhibitor sites I (green) and II (blue) and two phosphate sites (orange: the pseudo-product inhibitor in the active site, PP, and the allosteric site, aP). Circles indicate occupancy: open, empty; filled, bound ligand. Only one occupancy state corresponds to active enzyme. Inhibitor binding at I and II are mutually exclusive. Binding at I requires FPP occupancy (uncompetitive inhibition). Binding at II is mutually exclusive with FPP binding (competitive inhibition). Pseudo-product inhibition occurs only in the H state with occupied aP, because the affinity of aP is ˜500-fold higher than ΨP.

FIG. 5. Inhibition by 2f and 2q. Initial velocities, v, (circles) were extracted from progress curves (1) measured as a function of FPP (log scale) and different inhibitor concentrations (labeled, nM) at 0 mM (left column) or 10 mM (right column) phosphate, and fit (lines) to inhibition models (5) with binding polynomials for the different inhibition modes: 2f, mixed competitive and uncompetitive (8); 2q, uncompetitive (7). The values of the fit parameters and their error estimates were determined by bootstrapping (insets: distribution histograms of 10,000 trials); affinities are shown as their dissociation constants (nM): K_{F}, FPP affinity; K_{C}, competitive inhibitor affinity; K_{U}, uncompetitive inhibitor affinity). The 2f data set measured in the absence of phosphate was fit with ideal conditions (10); the other three required non-ideal treatment (11-13), because the total enzyme concentration, p_{T}, was ˜10 nM (see insets), close to value of at least one dissociation constant so that the ideality condition (9) does not hold.

FIG. 6. Electron density maps of 2q and 2f bound to CnFTase. Simulated annealing Fₒ-F_{c} electron density difference maps (gray, contoured at 4.0 σ; only positive densities are shown) revealed bound compounds, FPP (if present), Zn²⁺ (magenta), and sulfate. (A) 2q binds in the presence of FPP (7T08). (B) 2f displaces FPP (7T0A).

FIG. 7. Structures of inhibitor complexes. (A) 2q bound at site I (7T08) in an orientation similar to L-778, 123. R² butyl modification (dotted circle), present in most compounds, extends the contacts between the inhibitor and exit groove. The R¹-trifluoromethoxy group (dotted circle) forms halogen bonds (dotted lines) with terminal isoprene 3 of the FPP substrate, with the indole ring of Trp90 β, and within the inhibitor to the R³ chloride. These interactions strengthen the interactions of the inhibitor both with the bound substrate and with the protein. The internal halogen bond reduces flexibility. (B) 2f bound at site II (7T0A). The R¹-trifluoromethoxy swaps halogen bonds with the bound FPP terminal isoprene 3 at site I for bonds with Cys201 β and Cys272 β. The R⁴ bromide intercalates between Trp329 β and Tyr269 β, and forms a third halogen bond with Cys272 β. R² butyl group forms contacts in the exit groove. The sulfate occupies the bisphosphate-binding site and corresponds to the putative location of the phosphate pseudo-product inhibitor. (C) and (D) Superposition of 2f and 2q emphasize the differences in the locations of sites I and II, and the pivoting motion of the B ring around the catalytic Zn²⁺. If site II is occupied, the FPP substrate (outline in D) cannot bind. The R² butyl group binds to the exit groove in both locations but is less deeply inserted at site II. (D) Superposition of 2f and 2q emphasizes the rotation of the D ring around χ₂₃.

FIG. 8. The effects of inhibition mode and concentrations of substrate and enzyme on inhibitor efficacy. IC₅₀ values were calculated (3) for competitive (6) and uncompetitive (7) inhibition of a single-substrate enzyme. Substrate (K_{M}) and inhibitor affinities for both inhibition modes (K_{I}) were set to 1 (concentration units are the same for parameters and variables, and therefore omitted). Enzyme concentrations are presented as copy number per cell. Given a cell diameter of 10 μm (approximate size of C. neoformans), the volume is

$${V}_{c} = \frac{4}{3} π {r}^{3} = 523 {µm}^{3} = 5 \times {10}^{- 13} L .$$

The concentration of a single protein in that cell therefore is

$$\frac{1}{{N}_{A}} \times \frac{1}{{V}_{R}} = \frac{1}{6 \times {10}^{38}} \times 5 \times {10}^{- 13} \approx {10}^{- 10} = 0.1 nM .$$

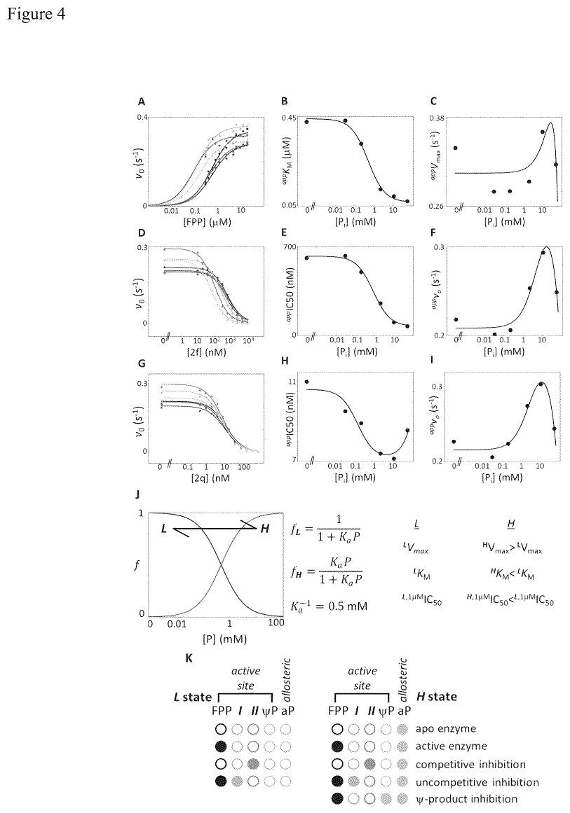

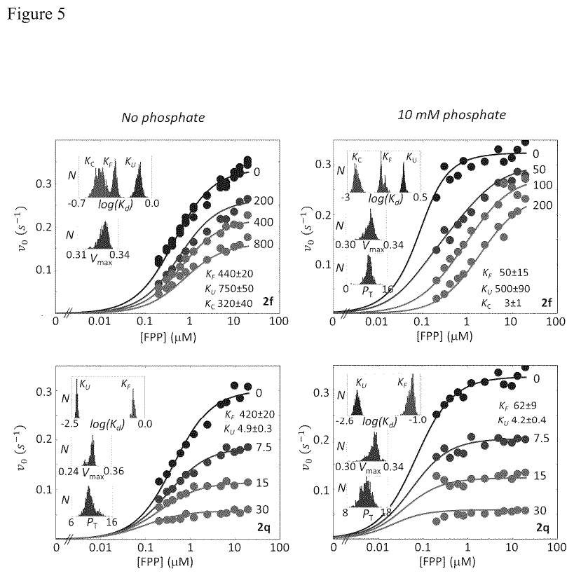

(A) The effect of substrate concentration ([S]) on IC₅₀ values for competitive (blue) and uncompetitive (green) inhibition at vanishingly small enzyme concentrations where the ideal numerical conditions apply (9). With these simulation parameters, the critical substrate concentration, S*, where the superior efficacy of these inhibition modes interchange corresponds to the K_{M} value of the enzyme. In general, S* depends on the three-way relationship between K_{M} and the two inhibition mode affinities. (B) The effect of enzyme concentration ([E]) on competitive inhibition IC₅₀ values, simulated using solvers for non-ideal numerical conditions (11-13). The phase space has been partitioned into four quadrants, using substrate and enzyme concentrations corresponding to K_{M} as the dividing lines: −/− (low substrate, low enzyme), −/+ (low substrate, high enzyme), +/− (high substrate, low enzyme), and +/+ (high substrate, high enzyme). The −/+ and +/− quadrants behave as the ideal case shown in panel (A). In the −/+ quadrant, the increase in available binding sites enables the competitive and active occupancies (FIG. 4K) to co-exist and rather than compete. Consequently, the inhibitor loses efficacy. In the +/+ quadrant the effects of increased protein and substrate concentration reinforce each other. Accordingly, competitive inhibition is most effective only at low substrate and protein concentrations. (C) The effect of enzyme concentration on uncompetitive inhibition IC₅₀ values. The −/+ and +/− quadrants also behave as the ideal case shown in panel (A). In the −/+ quadrant inhibitor efficacy improves because increased enzyme concentrations capture all free substrate. Given that the inhibitor binds only to the enzyme-substrate complex (FIG. 4K), IC₅₀ values approach K_{I}, as is the case in the +/− quadrant. In the +/+ quadrant, high substrate concentrations enable competition between the active and uncompetitive occupancies (FIG. 4K), thereby reducing inhibitor efficacy. Accordingly, uncompetitive inhibition is effective either at high substrate, or high enzyme concentrations. (D) Comparison of inhibitor effectiveness under plausible in vivo conditions. Contour plot of 2f IC₅₀ values (contour labels are μM) as a function of FPP concentration and enzyme copy number. The plot was generated by applying 5 with the mixed inhibition polynomial, Q_{M} (8), with affinities for the high-phosphate, H conformation (FIG. 5), and using solvers for non-ideal conditions 10-13. The efficacy quadrants are indicated. (E) Contour plot of 2q IC₅₀ values under plausible in vivo conditions, using the uncompetitive polynomial, Q_{U} (7). (F) Ratio of IC₅₀ (2q)/IC₅₀ (2f). Grey marks the region where 2f outperforms 2q at least two-fold in the joint −/− efficacy quadrant (it is asymmetrical because 2f has a mixed inhibition mode).

FIG. 9. shows the primers used for C. neoformans gene cloning.

FIG. 10. shows structural and functional properties of representative compounds.

FIG. 11. shows X-ray crystallography data collection and refinement statistics for representative compounds.

### DETAILED DESCRIPTION

Given the many challenges facing drug discovery, modifying high-affinity inhibitors that successfully passed stringent demands such as bioavailability and toxicity can be a fruitful strategy for discovering new inhibitors of similar targets in different pathogens (Iyer, K. R, et al., Nature Reviews Microbiology, 2021, 19, 454-466; and Privalsky, T. M, et al., Journal of the American Chemical Society, 2021, 143, 21127-21142).

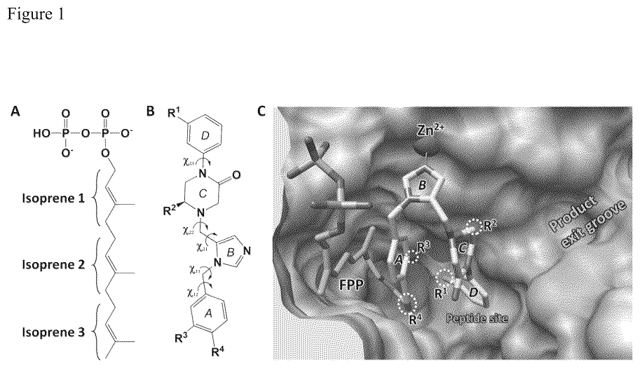

Human FTase, L-778,123 (FIG. 1B) was developed originally by Merck and Co, as an anti-tumor therapeutic that was well tolerated (Kohl, N. E, et al., Nat Med, 1995, 1, 792-7; Lobell, R. B, et al., Mol Cancer Ther, 2002, 1, 747-58; and Williams, T. M, et al., J Med Chem, 1999, 42, 3779-3784). It was subsequently found to exhibit modest inhibition of C. neoformans FTase (CnFTase), but it did not have antifungal activity (Hast, M. A, et al., J Biol Chem, 2011, 286, 35149-62). The structure of L-778,123 bound to CnFTase (Hast, M. A, et al., J Biol Chem, 2011, 286, 35149-62) was used to identify four sites in this inhibitor for construction of a focused collection of modifications, that provided derivatives that, unlike L-778, 123, exhibited potent antifungal activity.

Characterization of their structures and inhibition mechanisms revealed that the inhibitors bound at two, alternative, partially overlapping sites, accessed via different inhibitor conformations. At one site inhibitors bound uncompetitively, forming a farnesyl diphosphate-inhibitor complex: at the other competitively, displacing this substrate. Inhibitor affinities for these sites were determined by interactions that can be mediated only by the unique geometries of non-covalent halogen bonds. It was determined that the intracellular milieu has profound effects on enzyme activity and inhibition. First, it was determined that phosphate binding selects a higher-activity enzyme conformation. Second, only competitive inhibitors elicited antifungal activity despite having weaker affinity constants than uncompetitive inhibitors.

Statistical thermodynamics was used to develop computational tools that explicitly take into account enzyme as well as ligand and inhibitor concentrations to calculate the populations of inhibited enzyme for arbitrarily complex binding schemes. These tools demonstrated that only for a limited combination of protein, ligand and inhibitor concentrations competitive inhibition outperforms uncompetitive inhibition (at the low farnesyl transferase and FPP substrate concentrations expected to prevail in C. neoformans). These structural and mechanistic insights enabled the identification of lead compounds for urgently needed next-generation antifungals and establish design criteria that are generalizable for the development of other FTase inhibitors.

The following definitions are used, unless otherwise described.

The terms “treat”, “treatment”, or “treating” to the extent it relates to a disease or condition includes inhibiting the disease or condition, eliminating the disease or condition, and/or relieving one or more symptoms of the disease or condition. The terms “treat”, “treatment”, or “treating” also refer to both therapeutic treatment and/or prophylactic treatment or preventative measures, wherein the object is to prevent or slow down (lessen) an undesired physiological change or disorder, such as, for example, the development or spread of cancer. For example, beneficial or desired clinical results include, but are not limited to, alleviation of symptoms, diminishment of extent of disease or disorder, stabilized (i.e., not worsening) state of disease or disorder, delay or slowing of disease progression, amelioration or palliation of the disease state or disorder, and remission (whether partial or total), whether detectable or undetectable. “Treat”, “treatment”, or “treating,” can also mean prolonging survival as compared to expected survival if not receiving treatment. Those in need of treatment include those already with the disease or disorder as well as those prone to have the disease or disorder or those in which the disease or disorder is to be prevented. In one embodiment “treat”, “treatment”, or “treating” does not include preventing or prevention,

The phrase “therapeutically effective amount” or “effective amount” includes but is not limited to an amount of a compound of the that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “mammal” as used herein refers to humans, higher non-human primates, rodents, domestic, cows, horses, pigs, sheep, dogs and cats. In one embodiment, the mammal is a human. The term “patient” as used herein refers to any animal including mammals. In one embodiment, the patient is a mammalian patient In one embodiment, the patient is a human patient.

The compounds disclosed herein can also exist as tautomeric isomers in certain cases. Although only one delocalized resonance structure may be depicted, all such forms are contemplated within the scope of the invention.

It is understood by one skilled in the art that this invention also includes any compound claimed that may be enriched at any or all atoms above naturally occurring isotopic ratios with one or more isotopes such as, but not limited to, deuterium (²H or D). As a non-limiting example, a —CH₃ group may be substituted with —CD₃.

The pharmaceutical compositions of the invention can comprise one or more excipients. When used in combination with the pharmaceutical compositions of the invention the term “excipients” refers generally to an additional ingredient that is combined with the compound of formula (I) or the pharmaceutically acceptable salt thereof to provide a corresponding composition. For example, when used in combination with the pharmaceutical compositions of the invention the term “excipients” includes, but is not limited to: carriers, binders, disintegrating agents, lubricants, sweetening agents, flavoring agents, coatings, preservatives, and dyes.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or dis dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95 the absolute stereoisomer depicted. In another embodiment, the compound may be at least 99% the absolute stereoisomer depicted.

In cases where compounds are sufficiently basic or acidic, a salt of a compound of formula I can be useful as an intermediate for isolating or purifying a compound of formula I. Additionally, administration of a compound of formula I as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

In one embodiment, the competitive inhibitor of farnesyl transferase is competitive with respect to farnesyl disphosphate substrate

The invention will now be illustrated by the following non-limiting Examples.

### EXAMPLES

### Synthesis of Compounds

All chemicals and solvents were obtained from commercial sources in reagent grade and used without further purification. Chemicals 9a (Pingali, S. R. K, et al., Green Chem., 2011, 13, 928-933), 9c-d (Pingali, S. R. K, et al., Green Chem., 2011, 13, 928-933), 9h (Xu, X, et al., Eur. J. Med. Chem., 2018, 143, 1325-1344), and 9i (Betts, H. M, et al., J. Med. Chem., 2016, 59, 9422-9430) were prepared according to the reported literature.

Flash chromatography silica gel (60-200 mesh, 75-250 mm) was obtained from Mallinckrodt Inc.

¹H NMR, ¹³C NMR and ¹⁹F NMR spectra were recorded on a Bruker Avance 500 (500 MHz) spectrometer. Chemical shifts are reported in parts per million (ppm, δ) downfield from tetramethylsilane. Proton coupling patterns are described as singlet(s), doublet (d), triplet (t), quartet (q), multiplet (m), and broad (br). High-resolution mass spectrometry (HRMS) measurements were performed using a Bruker BioTOF II (ESI-TOF) instrument in electrospray ionization (ESI) mode. PEG or PPG was used as an internal standard/calibrant. Samples were introduced as solutions in CH₃OH (CD₃OD) or, when CH₃OH-solubility was poor, in methylene chloride (CD₂Cl₂).

High-performance liquid chromatography (HPLC) analysis (analytical and preparative) was performed using a Beckman model 125/166 instrument, equipped with a UV detector and C18 columns (Varian Microsorb-MV, 5 μm, 4.6×250 mm and Phenomenex Luna, 10 μm, 10×250 mm, respectively) or an Agilent 1100 series instrument, equipped with a diode-array detector and C18 columns (Agilent ZORBA X 300SB—C18 5 μM 9.4×250 mm, or Agilent Pursuit C18 5 μM 250×21.2 mm, respectively), and using an H₂O/CH₃CN system containing trifluoroacetic acid (TFA) consisting of solvent A (H₂O with 0.1% TFA) and solvent B (CH₃CN with 0.1% TFA).

### Examples 1-9

Synthetic route for the preparation of Compounds 2d-i and 20-q.

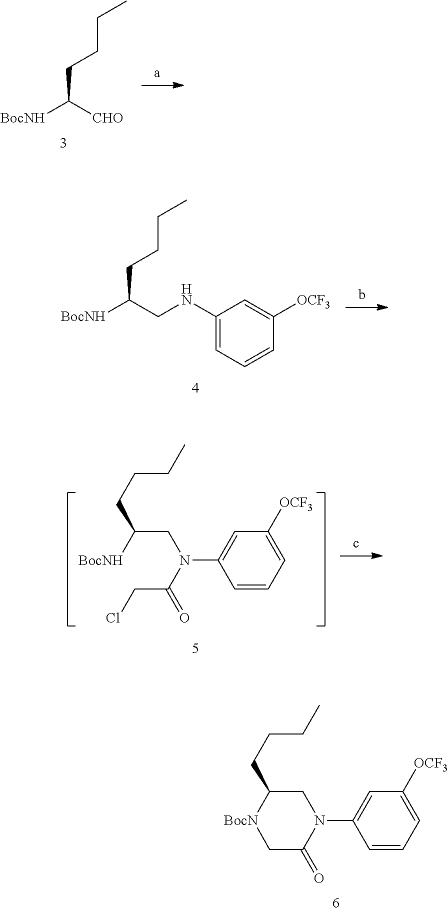

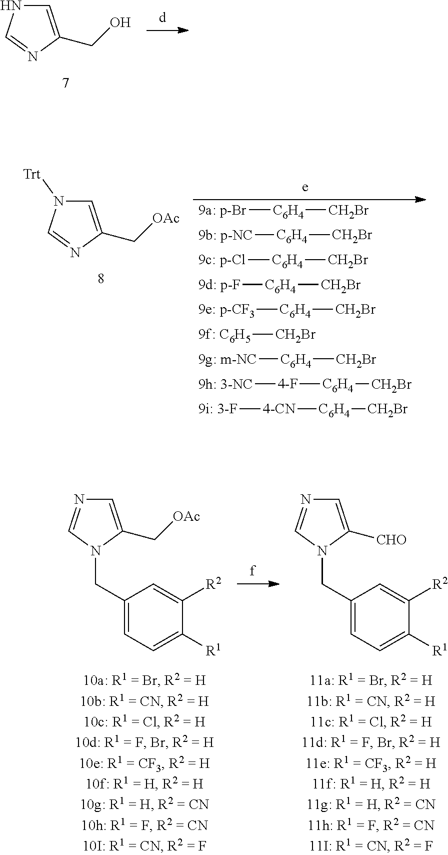

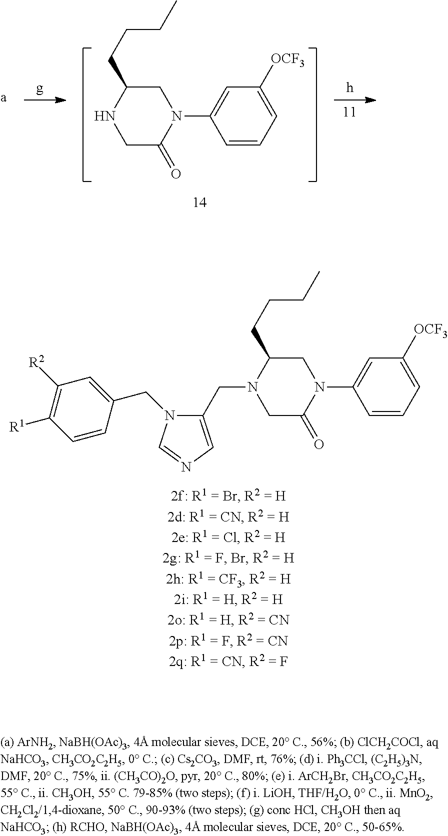

### Example 1. Synthesis of(S)-4-((5-((2-butyl-5-oxo-4-(3-(trifluoromethoxy)-phenyl)piperazin-1-yl)methyl)-1H-imidazol-1-yl)methyl)benzonitrile (2d)

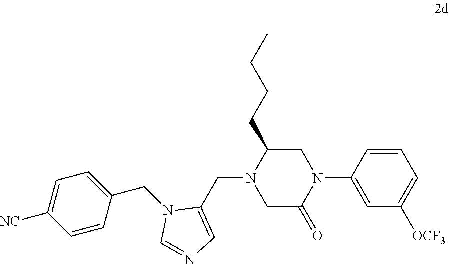 [SMILES: CCCC[C@H]1CN(c2cccc(OC(F)(F)F)c2)C(=O)CN1Cc1cncn1Cc1ccc(C#N)cc1]

Compound 2d was prepared via the synthetic procedure described for compound 2f (see below), except that compound 11b was used instead of compound 11a (Suryadevara, P. K, et al., J. Med. Chem., 2009, 52, 3703-3715). Yield: 42% (43 mg on a 0.2 mmol reaction scale). Colorless oil. Purity: 97% by HPLC. ¹H NMR (CD₃OD, 500 MHZ) δ 9.12 (s, 1H), 7.83 (d, J=8.3 Hz, 2H), 7.73 (s, 1H), 7.56 (t, J=8.7 Hz, 1H), 7.46 (d, J=8.2 Hz, 2H), 7.29-7.27 (m, 3H), 5.74 (s, 2H), 3.94 (d, J=14.7 Hz, 1H), 3.83 (d, J=14.6 Hz, 1H), 3.72 (dd, J=4.2, 12.2 Hz, 1H), 3.45 (dd, J=5.0, 12.2 Hz, 1H), 3.34 (d, J=17.5 Hz, 1H), 3.24 (d, J=17.5 Hz, 1H), 3.04-2.99 (m, 1H), 1.71-1.64 (m, 1H), 1.59-1.51 (m, 1H), 1.41-1.27 (m, 4H), 0.93 (t, J=7.3 Hz, 3H). ¹³C{¹H} NMR (CD₃OD 125 MHZ) δ 169.8, 151.5 (q, J=3.2 Hz), 145.3, 142.2, 139.8, 135.0, 133.3, 132.6, 129.8, 126.4, 123.7 (q, J=255.1 Hz), 122.3, 121.6, 121.0, 119.9, 114.5, 58.5, 54.2, 54.0, 52.1, 47.5, 30.6, 28.2, 24.6, 15.1. ¹⁹F{¹H} NMR (CD₃OD, 470 MHZ) δ −59.5. HRMS (ESI-TOF): Calcd for (C₂₇H₂₉F₃N₅O₂)⁺ 512.2268; found: 512.2285.

The intermediate compound 6 was prepared as follows.

### a. Synthesis of tert-Butyl(S)-(1-((3-(trifluoromethoxy)phenyl)amino)hexan-2-yl)-carbamate (4)

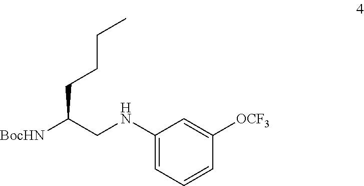 [SMILES: CCCC[C@@H](CNc1cccc(OC(F)(F)F)c1)NC(=O)OC(C)(C)C]

A solution of aldehyde 3 (645 mg, 3 mmol; Douangamath, A, et al., J. Med. Chem., 2004, 47, 1325-1328) in ClCH₂CH₂Cl (6 mL, 0.5 M) and aniline (637 mg, 3.6 mmol) was stirred for 1 h, and NaBH(OAc)₃ (1.27 g, 6 mmol), followed by catalytic glacial acetic acid (2 drops), was added. The reaction was allowed to stir at rt for 48 h, then added to CH₂Cl₂ (10 mL) and saturated aqueous Na₂CO₃ (5 mL), and the mixture was extracted with EtOAc (3×50 mL). The organic phase was washed with 1 M HCl (50 mL), brine (50 mL), dried with anhydrous MgSO₄, evaporated to dryness, and purified by column chromatography (Hexanes/EtOAc, 8:1, v/v) to yield the desired product, 4, as a pale-yellow oil in 56% yield (633 mg). ¹H NMR (CDCl₃, 500 MHz) δ 7.12 (t, J=8.1 Hz, 1H), 6.52-6.48 (m, 2H), 6.38 (s, 1H), 4.45 (bs, 2H), 3.81 (bs, 1H), 3.22-3.19 (m, 1H), 3.02-2.98 (m, 1H), 1.58-1.33 (m, 6H), 1.44 (s, 9H), 0.91 (t, J=6.9 Hz, 3H). ¹⁹F{¹H} NMR (CDCl₃, 470 MHZ) δ −59.5. HRMS (ESI-TOF): Calcd for (C₁₈H₂₈F₃N₂O₃)⁺ 377.2047; found: 377.2058.

### b. Synthesis of tert-Butyl(S)-2-butyl-5-oxo-4-(3-(trifluoromethoxy)phenyl) piperazine-1-carboxylate (6)

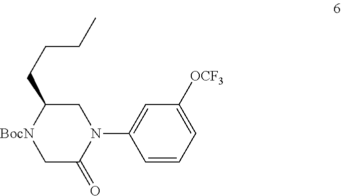 [SMILES: CCCC[C@H]1CN(c2cccc(OC(F)(F)F)c2)C(=O)CN1C(=O)OC(C)(C)C]

Aniline intermediate 4 (752 mg, 2 mmol) was dissolved in a mixture of EtOAc (0.2 M) and sat. NaHCO₃ solution (0.1 M), then cooled to 0° C. With vigorous stirring, chloroacetyl chloride (336 mg, 3 mmol) was added dropwise. After 3 hours, the reaction was diluted with H₂O (50 mL) and EtOAc (50 mL). The organic phase was washed with H₂O (100 mL), brine (100 mL), dried with anhydrous MgSO₄, evaporated to dryness to yield the desired intermediates. The crude product was used for the next step without purification due to its limited stability.

To a DMF solution (0.2 M) of the above crude product was added Cs₂CO₃ (650 mg, 2 mmol), the reaction was allowed to stir at rt for 24 hours, then added to 1 M HCl (100 mL), and extracted with EtOAc (3×50 mL). The organic phase was washed with H₂O (50 mL), brine (50 mL), dried with anhydrous MgSO₄, evaporated to dryness, and purified by column chromatography (Hexanes/EtOAc, 6:1, v/v) to yield the desired product, 6 (667 mg), as a pale-yellow oil in 76% yield. ¹H NMR (CDCl₃, 500 MHZ) δ 7.45 (t, J=8.1 Hz, 1H), 7.25 (d, J=8.8 Hz, 1H), 7.18 (s, 1H), 7.16 (d, J=8.4 Hz, 1H), 4.48 (bs, 2H), 4.06 (dd, J=4.0, 12.2 Hz, 1H), 3.90 (d, J=18.8 Hz, 1H), 3.47 (d, J=12.2 Hz, 1H), 1.87-1.79 (m, 1H), 1.66-1.62 (m, 1H), 1.52 (s, 9H), 1.45-1.26 (m, 4H), 0.94 (t, J=7.0 Hz, 3H). ¹³C{¹H} NMR (CDCl₃, 125 MHZ) δ 165.6, 153.7, 149.5 (q, J=3.0 Hz), 143.3, 130.3, 123.7, 120.4 (q, J=255.0 Hz), 119.3, 118.4, 80.9, 53.1, 29.4, 28.5, 28.1, 22.4, 13.9. ¹⁹F{¹H} NMR (CDCl₃, 470 MHz) δ −57.8. HRMS (ESI-TOF): Calcd for (C₂₀H₂₇F₃N₂NaO₄)⁺ 439.1815; found: 439.1817.

The intermediate compound 11b was prepared as follows.

### c. Synthesis of (1-Trityl-1H-imidazol-4-yl)methyl acetate (8; Millet, R, et al., J. Med. Chem. 2004, 47, 6812-6820)

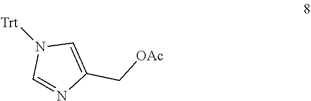

Trityl chloride (12 g, 40.7 mmol), and NEt₃ (15.5 mL, 111 mmol) were added to a suspension of 1H-4-imidazolylmethanol hydrochloride (7, 5 g, 37 mmol) in DMF (100 mL). After stirring at 45° C. for 12 h, the solution was poured into H₂O (1.5 L). The precipitate was filtered, and washed first with H₂O and then with Et₂O, and then recrystallized from dioxane to give an intermediate alcohol as a white solid in 75% yield. To a solution of that compound in pyridine (60 mL) was added Ac₂O (5 mL). The solution was stirred at rt for 24 h and then concentrated under reduced pressure. The solid obtained was recrystallized from cyclohexane to give 8 (11.3 g) as a white powder in 80% yield. ¹H NMR (CDCl₃, 500 MHZ) δ 7.42 (s, 1H), 7.35-7.32 (m, 9H), 7.14-7.12 (m, 6H), 6.87 (s, 1H), 5.01 (s, 2H), 2.07 (s, 3H).

### d. Synthesis of 4-((5-Formyl-1H-imidazol-1-yl)methyl)benzonitrile (11b, from 13, Suryadevara, P. K, et al., J. Med. Chem., 2009, 52, 3703-3715; and J. Med. Chem. 2002, 45, 177-188)

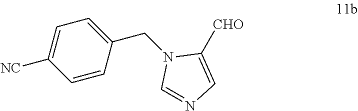 [SMILES: N#Cc1ccc(Cn2cncc2C=O)cc1]

Compound 11b was prepared via the synthetic procedure for compound 11a (from 13), except that compound 9b was used instead of compound 9a. Yield: 79% (250 mg). Pale yellow solid. ¹H NMR (CDCl₃, 500 MHZ) δ 9.74 (s, 1H), 7.87 (s, 1H), 7.77 (s, 1H), 7.63 (d, J=8.1 Hz, 2H), 7.25 (d, J=8.1 Hz, 2H), 5.58 (s, 2H).

### Example 2. Synthesis of(S)-(5-Butyl-4-((1-(4-chlorobenzyl)-1H-imidazol-5-yl)methyl)-1-(3-(trifluoromethoxy)phenyl)piperazin-2-one (2e)

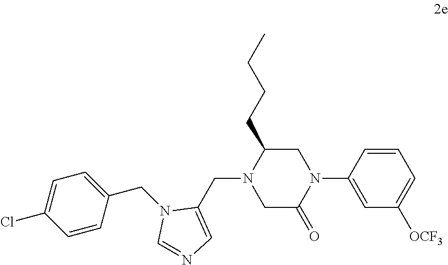 [SMILES: CCCC[C@H]1CN(c2cccc(OC(F)(F)F)c2)C(=O)CN1Cc1cncn1Cc1ccc(Cl)cc1]

Compound 2e was prepared via the synthetic procedure described for compound 2f, except that compound 11c (Suryadevara, P. K, et al., J. Med. Chem., 2009, 52, 3703-3715) was used instead of compound 11a. Yield: 52% (54 mg on a 0.2 mmol reaction scale). Colorless oil. Purity: 99% by HPLC. ¹H NMR (CD₃OD, 500 MHZ) δ 9.09 (s, 1H), 7.70 (s, 1H), 7.63 (d, J=8.2 Hz, 2H), 7.46 (t, J=7.6 Hz, 2H), 7.35 (d, J=7.4 Hz, 1H), 7.26-7.22 (m, 3H), 5.60 (s, 2H), 3.94 (d, J=14.6 Hz, 1H), 3.85 (d, J=14.6 Hz, 1H), 3.69 (dd, J=4.0, 12.4 Hz, 1H), 3.42 (dd, J=4.6, 12.4 Hz, 1H), 3.41 (d, J=17.4 Hz, 1H), 3.26 (d, J=17.4 Hz, 1H), 3.03-2.99 (m, 1H), 1.73-1.67 (m, 1H), 1.61-1.54 (m, 1H), 1.41-1.28 (m, 4H), 0.94 (t, J=6.9 Hz, 3H). ¹³C{¹H} NMR (CD₃OD 125 MHz) δ 166.5, 149.8 (q, J=3.0 Hz), 143.0, 137.6, 136.0, 131.6, 130.7, 130.2, 129.7, 128.9, 124.2, 121.6 (q, J=260.3 Hz), 119.9, 118.7, 56.4, 53.4, 52.4, 50.7, 46.6, 29.0, 26.0, 23.0, 14.2. ¹⁹F{¹H} NMR (CD₃OD, 470 MHZ) δ −59.5. HRMS (ESI-TOF): Calcd for (C₂₆H₂₉ClF₃N₄O₂)⁺ 521.1926; found: 521.1953.

The intermediate compound 11c was prepared as follows.

### a. Synthesis of 1-(4-Chlorobenzyl)-1H-imidazole-5-carbaldehyde (11c, from 13 Suryadevara, P. K, et al., J. Med. Chem., 2009, 52, 3703-3715)

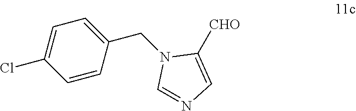 [SMILES: O=Cc1cncn1Cc1ccc(Cl)cc1]

Compound 11c was prepared via the synthetic procedure for compound 11a (from 13), except that compound 9c (Pingali, S. R. K, et al., Green Chem., 2011, 13, 928-933) was used instead of compound 9a. Yield: 83% (274 mg). Pale yellow solid. ¹H NMR (CDCl₃, 500 MHz) δ 9.75 (s, 1H), 7.84 (s, 1H), 7.71 (s, 1H), 7.31 (d, J=8.1 Hz, 2H), 7.15 (d, J=8.1 Hz, 2H), 5.48 (s, 2H).

### Example 3. Synthesis of(S)-4-((1-(4-Bromobenzyl)-1H-imidazol-5-yl)methyl)-5-butyl-1-(3-(trifluoromethoxy)phenyl)piperazin-2-one (2f)

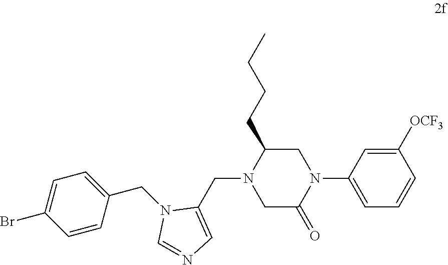 [SMILES: CCCC[C@H]1CN(c2cccc(OC(F)(F)F)c2)C(=O)CN1Cc1cncn1Cc1ccc(Br)cc1]

To a THF solution (0.5 mL) of Compound 6 (83.2 mg, 0.2 mmol) was added concentrated HCl aq (0.5 mL), and the mixture was maintained at rt for 12 hours. The reaction mixture was poured into saturated aqueous NaHCO₃ solution and extracted with EtOAc (3×50 mL). The combined extracts were dried over anhydrous MgSO₄, filtered, and concentrated under reduced pressure. To the crude product above dissolved in ClCH₂CH₂Cl (3 mL) was added 11a (43.8 mg, 0.17 mmol), NaBH (OAc) 3 (90.1 mg, 0.425 mmol), 4 Å sieves (broken, 200 mg), and AcOH (1 drop), and the mixture allowed to react at room temperature for 24 hours at which point it was quenched by addition of aqueous NaHCO₃ solution (10 mL), and filtered through celite to remove the molecular sieves. The filtrate was poured into H₂O, extracted with EtOAc (3×50 mL), and the combined extracts were dried over anhydrous MgSO₄, filtered, and concentrated under reduced pressure. The final product was purified using preparative HPLC (Solvent A: H₂O—0.1% TFA, Solvent B: CH₃CN—0.1% TFA, Solvent gradient: 0% B for 5 minutes followed by 0-100% B over 60 min) to afford pure Compound 2f (50.8 mg) as a colorless oil in 45% yield. Purity: 99% by HPLC. ¹H NMR (CD₃OD, 500 MHz) δ 9.09 (s, 1H), 7.70 (s, 1H), 7.62 (d, J=8.4 Hz, 2H), 7.56 (dd, J=8.5 Hz, 1H), 7.31 (d, J=8.6 Hz, 1H), 7.30 (s, 1H), 7.27 (d, J=8.6 Hz, 1H), 7.23 (d, J=8.4 Hz, 2H), 5.60 (s, 2H), 3.94 (d, J=14.6 Hz, 1H), 3.84 (d, J=14.6 Hz, 1H), 3.74 (dd, J=4.1, 12.2 Hz, 1H), 3.46 (dd, J=4.8, 12.2 Hz, 1H), 3.37 (d, J=17.4 Hz, 1H), 3.27 (d, J=17.5 Hz, 1H), 3.02 (m, 1H), 1.72-1.66 (m, 1H), 1.60-1.53 (m, 1H), 1.39-1.29 (m, 4H), 0.92 (dd, J=6.95 Hz, 3H). ¹³C{¹H} NMR (CD₃OD 125 MHZ) δ 167.6, 146.2 (q, J=3.0 Hz), 143.1, 137.2, 133.6, 132.0, 130.9, 130.3, 128.8, 124.3, 122.3, 121.5 (q, J=256.2 Hz), 119.8, 119.3, 118.8, 56.1, 52.0, 51.8, 49.8, 45.3, 28.4, 25.8, 22.4. ¹⁹F{¹H} NMR (CD₃OD, 470 MHz) δ −59.5. HRMS (ESI-TOF): Calcd for (C₂₆H₂₉BrF₃N₄O₂)⁺ 565.1420; found: 565.1423.

The intermediate compound 11a was prepared as follows.

### a. Synthesis of (1-(4-Bromobenzyl)-1H-imidazol-5-yl)methyl acetate (10a)

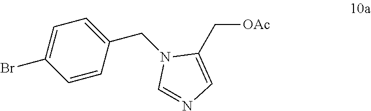 [SMILES: CC(=O)OCc1cncn1Cc1ccc(Br)cc1]

Compound 8 (1.9 g, 5 mmol) was added to a solution of commercial benzyl bromide derivative, 9a (1.36 g, 5.5 mmol; Pingali, S. R. K, et al., Green Chem., 2011, 13, 928-933) in EtOAc (20 mL). The mixture was stirred at 55° C. for 48 hours, and after being cooled to room temperature, the resulting imidazolium salt was isolated by filtration and washed with EtOAc (3×80 mL). The resulting imidazolium salt was dissolved in CH₃OH (100 mL) and stirred at reflux for 12 hours. The solvent was evaporated under reduced pressure, and the residue was triturated with a 5% citric acid solution. The solution was filtered and adjusted to pH 6 with 5% Na₂CO₃. The resulting precipitate was filtered, washed with Et₂O, to give 10a (1.3 g) as a white solid in 85% yield. ¹H NMR (CD₃OD, 500

### b. Synthesis of 1-(4-Bromobenzyl)-1H-imidazole-5-carbaldehyde (11a (Suryadevara, P. K, et al., J. Med. Chem., 2009, 52, 3703-3715) from 10a)

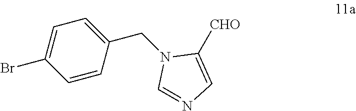 [SMILES: O=Cc1cncn1Cc1ccc(Br)cc1]

Compound 10a (1.54 g, 5 mmol) was dissolved in THF (20 mL), 2 N LiOH (20 mmol, 20 mL) was added, and the resulting mixture was stirred at 0° C. for 2 hours. The solution was neutralized with 2 N HCl to pH 7, and the solvents were removed under reduced pressure, the products were passed through a thin pad of SiO₂ using CH₃OH/CH₂Cl₂ (1/20, v/v) to remove the resulting inorganic salts, and the solvents were removed under reduced pressure. To a solution of the resulting unpurified imidazole in dioxane and CH₂Cl₂ (15 mL, 1/1, v/v) was added MnO₂ (4.35 g, 50 mmol). The suspension was stirred at 45° C. for 12 hours, then the MnO₂ was filtered and washed with CH₂Cl₂. The resulting solution was evaporated under reduced pressure, and the residue was purified by column chromatography on silica gel using CH₂Cl₂/CH₃OH as eluent to give the product (11a, 1.18 g) in 90% yield as a white powder. ¹H NMR (CDCl₃, 500 MHZ) δ 9.75 (s, 1H), 7.84 (s, 1H), 7.71 (s, 1H), 7.47 (d, J=8.1 Hz, 2H), 7.08 (d, J=8.1 Hz, 2H), 5.47 (s, 2H).

### c. Alternate Synthesis of 1-(4-Bromobenzyl)-1H-imidazole-5-carbaldehyde (11a from 13)

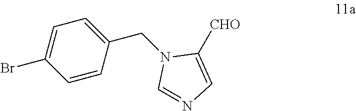 [SMILES: O=Cc1cncn1Cc1ccc(Br)cc1]

A solution of compound 13 (507 mg, 1.5 mmol) in CH₃CN (10 mL) was treated with alkyl bromide 9a (372 mg, 1.5 mmol) at room temperature and heated to 60° C. and stirred overnight under nitrogen. The reaction mixture was cooled and concentrated under vacuum, and the resulting paste was triturated with acetone (20 mL) and stirred for 2-3 hours. The resulting solid was isolated by filtration and extracted with CH₂Cl₂ (2×25 mL) and washed with saturated NaHCO₃ (25 mL). The organic layers were combined, dried over Na₂SO₄, and concentrated to give 11a (362 mg) in 91% yield.

### d. Synthesis of 1-Triphenylmethyl-4-imidazole carboxaldehyde (13)

To a 1 L three-necked round-bottomed flask with an addition funnel was added 3H-imidazole-4-carbaldehyde (1.2 g, 12.5 mol), trityl chloride (3.8 g, 13.7 mol), and CH₃CN (50 mL). The mixture was stirred at rt to give a slurry. Triethylamine (3 mL, 22 mol) was added dropwise over 20 min and the resulting reaction mixture was stirred at rt for 20 h. Hexane (20 mL) and H₂O (100 mL) were added. The slurry was stirred for 30 min and filtered. The cake was washed with H₂O (3×50 mL) and dried in a vacuum oven at 50° C. for 20 h to give 13 as a white solid (3.9 g) in 94% yield. ¹H NMR (CDCl₃) δ 9.81 (s, 1H), 7.54 (s, 1H), 7.46 (s, 1H), 7.29 (m, 10H), 7.04 (m, 5H).

### Example 4. Synthesis of(S)-5-butyl-4-((1-(4-fluorobenzyl)-1H-imidazol-5-yl)methyl)-1-(3-(trifluoromethoxy)phenyl)piperazin-2-one (2g)

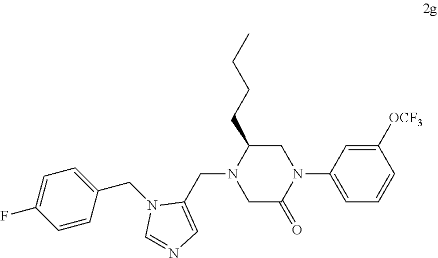 [SMILES: CCCC[C@H]1CN(c2cccc(OC(F)(F)F)c2)C(=O)CN1Cc1cncn1Cc1ccc(F)cc1]

Compound 2g was prepared via the synthetic procedure described for compound 2f, except that compound 11d was used instead of compound 11a. Yield: 50% (63 mg on a 0.25 mmol reaction scale). Colorless oil. Purity: 99% by HPLC. ¹H NMR (CD₃OD, 500 MHZ) δ 9.03 (s, 1H), 7.68 (s, 1H), 7.56 (t, J=8.1 Hz, 1H), 7.38-7.36 (m, 2H), 7.33-7.26 (m, 3H), 7.20 (t, J=8.7 Hz, 2H), 5.60 (s, 2H), 3.95 (d, J=14.6 Hz, 1H), 3.85 (d, J=14.5 Hz, 1H), 3.81 (dd, J=4.0, 12.2 Hz, 1H), 3.51 (dd, J=4.9, 12.2 Hz, 1H), 3.40 (d, J=17.4 Hz, 1H), 3.30 (d, J=17.5 Hz, 1H), 3.08-3.04 (m, 1H), 1.76-1.69 (m, 1H), 1.63-1.55 (m, 1H), 1.41-1.30 (m, 4H), 0.93 (t, J=7.0 Hz, 3H). ¹³C{¹H} NMR (CD₃OD 125 MHz) δ 166.1, 164.1, 151.5 (q, J=3.2 Hz), 145.3, 139.2, 133.1, 132.6, 132.3 (d, J=2.9 Hz), 131.6 (d, J=8.2 Hz), 126.5, 122.7 (q, J=256.1 Hz), 122.1, 121.6, 121.1, 118.0 (d, J=22.1 Hz), 58.4, 54.3, 54.1, 52.0, 47.5, 30.6, 28.2, 24.6, 15.1. ¹⁹F{¹H} NMR (CD₃OD, 470 MHz) δ −59.5, −114.8. HRMS (ESI-TOF): Calcd for (C₂₆H₂₉F₄N₄O₂)⁺505.2221; found: 505.2257.

The intermediate compound 11d was prepared as follows.

### a. Synthesis of (1-(4-Fluorobenzyl)-1H-imidazol-5-yl)methyl acetate (10d)

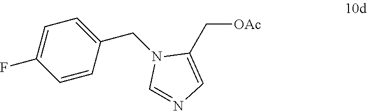 [SMILES: CC(=O)OCc1cncn1Cc1ccc(F)cc1]

Compound 10d was prepared via the synthetic procedure reported for compound, except that compound 9d was used instead of compound 9a. Yield: 79% (983 mg on a 5 mmol reaction scale). White powder. ¹H NMR (CD₃OD, 500 MHz) δ 8.96 (s, 1H), 7.70 (s, 1H), 7.39 (m, 2H), 7.20 (m, 2H), 5.52 (s, 2H), 5.16 (s, 2H), 1.95 (s, 3H). HRMS (ESI-TOF): Calcd for (C₁₃H₁₄FN₂O₂)⁺ 249.1034; found: 249.1036.

### b. Synthesis of 1-(4-Fluorobenzyl)-1H-imidazole-5-carbaldehyde (11d, from 10d; Suryadevara, P. K, et al., J. Med. Chem. 2009, 52, 3703-3715)

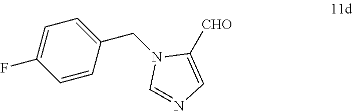 [SMILES: O=Cc1cncn1Cc1ccc(F)cc1]

Compound 11d was prepared via the synthetic procedure reported for compound 11a, except that compound 10d was used instead of compound 10a. Yield: 93% (948 mg). White powder. ¹H NMR (CDCl₃, 500 MHZ) δ 9.75 (s, 1H), 7.83 (s, 1H), 7.70 (s, 1H), 7.23-7.20 (m, 2H), 7.05-7.02 (m, 2H), 5.48 (s, 2H).

### c. Alternate Synthesis of 1-(4-Fluorobenzyl)-1H-imidazole-5-carbaldehyde (11d from 13)

Compound 11d was prepared via the synthetic procedure for compound 11a (from 13, except that compound 9d (Pingali, S. R. K, et al., Green Chem., 2011, 13, 928-933) was used instead of compound 9a. Yield: 91% (278 mg). White solid. ¹H NMR (CDCl₃, 500 MHZ) δ 9.75 (s, 1H), 7.83 (s, 1H), 7.70 (s, 1H), 7.23-7.20 (m, 2H), 7.05-7.02 (m, 2H), 5.48 (s, 2H).

### Example 5. Synthesis of(S)-5-Butyl-1-(3-(trifluoromethoxy)phenyl)-4-((1-(4-(trifluoromethyl)benzyl)-1H-imidazol-5-yl)methyl)piperazin-2-one (2h)

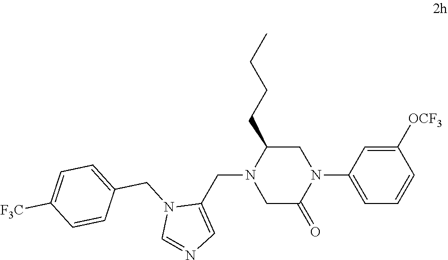 [SMILES: CCCC[C@H]1CN(c2cccc(OC(F)(F)F)c2)C(=O)CN1Cc1cncn1Cc1ccc(C(F)(F)F)cc1]

Compound 2h was prepared via the synthetic procedure described for compound 2f, except that compound 11e was used instead of compound 11a. Yield: 52% (58 mg on a 0.2 mmol reaction scale). Colorless oil. Purity: 99% by HPLC. ¹H NMR (CD₃OD, 500 MHZ) δ 9.15 (s, 1H), 7.77 (t, J=8.2 Hz, 2H), 7.73 (s, 1H), 7.54 (t, J=9.1 Hz, 1H), 7.48 (d, J=8.1 Hz, 2H), 7.28 (s, 1H), 7.27 (t, J=7.5 Hz, 2H), 5.75 (s, 2H), 3.95 (d, J=14.7 Hz, 1H), 3.84 (d, J=14.6 Hz, 1H), 3.73 (dd, J=4.0, 12.2 Hz, 1H), 3.47 (dd, J=4.9, 12.2 Hz, 1H), 3.38 (d, J=17.5 Hz, 1H), 3.28 (d, J=17.5 Hz, 1H), 3.08-3.01 (m, 1H), 1.69-1.64 (m, 1H), 1.59-1.51 (m, 1H), 1.38-1.27 (m, 4H), 0.91 (t, J=7.2 Hz, 3H). ¹³C{¹H} NMR (CD₃OD 125 MHZ) δ 169.9, 151.5 (q, J=3.2 Hz), 145.3, 141.1, 139.7, 133.2, 122.7 (q, J=32.2 Hz), 132.6, 129.7, 128.1 (q, J=3.6 Hz), 126.5, 126.2 (q, J=271.2 Hz), 122.7 (q, J=256.2 Hz), 122.1, 121.5, 121.0, 58.5, 54.3, 54.0, 52.1, 47.6, 30.6, 28.2, 24.6, 15.1. ¹⁹F{¹H} NMR (CD₃OD, 470 MHz) δ −59.5, −64.2. HRMS (ESI-TOF): Calcd for (C₂₇H₂₉F₆N₄O₂)⁺ 555.2189; found: 555.2213.

The intermediate compound 11e was prepared as follows.

### a. Synthesis of 1-(4-(Trifluoromethyl)benzyl)-1H-imidazole-5-carbaldehyde (11e, from 13; Millet, R, et al., J. Med. Chem. 2004, 47, 6812-6820)

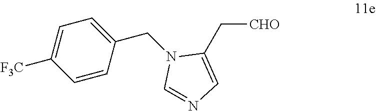 [SMILES: O=CCc1cncn1Cc1ccc(C(F)(F)F)cc1]

Compound 11e was prepared via the synthetic procedure for compound 11a (from 13) described above, except that compound 9e was used instead of compound 9a. Yield: 70% (267 mg). Pale yellow solid. ¹H NMR (CDCl₃, 500 MHz) δ 9.75 (s, 1H), 7.86 (s, 1H), 7.76 (s, 1H), 7.60 (d, J=8.0 Hz, 1H), 7.29 (d, J=8.0 Hz, 1H), 5.58 (s, 2H).

### Example 6. Synthesis of(S)-4-((1-Benzyl-1H-imidazol-5-yl)methyl)-5-butyl-1-(3-(trifluoromethoxy)phenyl)piperazin-2-one (2i)

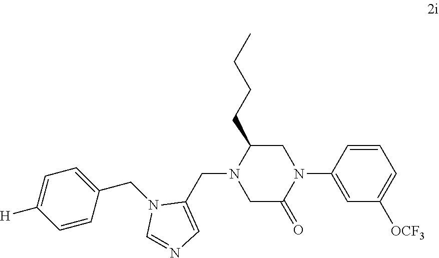 [SMILES: CCCC[C@H]1CN(c2cccc(OC(F)(F)F)c2)C(=O)CN1Cc1cncn1Cc1ccccc1]

Compound 2i was prepared via the synthetic procedure described for compound 2f, except that compound 11f was used instead of compound 11a. Yield: 41% (40 mg on a 0.2 mmol reaction scale). Colorless oil. Purity: 98% by HPLC. ¹H NMR (CD₃OD, 500 MHz) δ 9.05 (s, 1H), 7.69 (s, 1H), 7.56 (t, J=8.9 Hz, 2H), 7.50-7.41 (m, 3H), 7.34-7.26 (m, 4H), 5.64 (s, 2H), 3.93 (d, J=14.8 Hz, 1H), 3.83 (d, J=14.6 Hz, 1H), 3.77 (dd, J=4.0, 12.2 Hz, 1H), 3.48 (dd, J=5.0, 12.1 Hz, 1H), 3.39 (d, J=17.5 Hz, 1H), 3.28 (d, J=17.4 Hz, 1H), 3.07-3.01 (m, 1H), 1.77-1.66 (m, 1H), 1.63-1.52 (m, 1H), 1.41-1.29 (m, 4H), 0.93 (t, J=7.1 Hz, 3H). ¹³C{¹H} NMR (CD₃OD 125 MHZ) δ 170.0, 162.5, 151.5 (q, J=3.2 Hz), 145.4, 138.9, 133.3, 132.6, 131.0, 127.9, 126.6, 121.9, 121.6, 121.1 (q, J=258.6 Hz), 116.6, 56.4, 54.3, 52.4, 50.7, 47.6, 30.6, 25.1, 24.6, 15.1. ¹⁹F{¹H} NMR (CD₃OD, 470 MHz) δ −59.5. HRMS (ESI-TOF): Calcd for (C₂₆H₃₀F₃N₄O₂)⁺ 487.2315; found: 487.2329.

The intermediate compound 11f was prepared as follows.

### a. Synthesis of 1-Benzyl-1H-imidazole-5-carbaldehyde (11f, from 13; Millet, R, et al., J. Med. Chem., 2004, 47, 6812-6820)

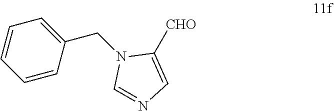 [SMILES: O=Cc1cncn1Cc1ccccc1]

Compound 11f was prepared via the synthetic procedure for compound 11a (from 13) described above, except that compound 9f was used instead of compound 9a. Yield: 75% (209 mg). Pale yellow oil. ¹H NMR (CDCl₃, 500 MHz) δ 9.76 (s, 1H), 7.86 (s, 1H), 7.76 (s, 1H), 7.37-7.29 (m, 3H), 7.22-7.20 (m, 2H), 5.52 (s, 2H).

### Example 7. Synthesis of(S)-3-((5-((2-Butyl-5-oxo-4-(3-(trifluoromethoxy)-phenyl)piperazin-1-yl)methyl)-1H-imidazol-1-yl)methyl)benzonitrile (20)

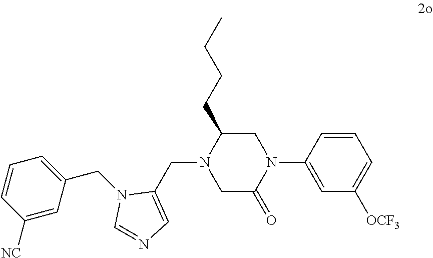 [SMILES: CCCC[C@H]1CN(c2cccc(OC(F)(F)F)c2)C(=O)CN1Cc1cncn1Cc1cccc(C#N)c1]

Compound 20 was prepared via the synthetic procedure described for compound 2f, except that compound 11g was used instead of compound 11a. Yield: 45% (48 mg on a 0.21 mmol reaction scale). Colorless oil. Purity: 99% by HPLC. ¹H NMR (CD₃OD, 500 MHZ) δ 9.11 (s, 1H), 7.80 (d, J=7.4 Hz, 1H), 7.75 (s, 1H), 7.72 (s, 1H), 7.67-7.62 (m, 2H), 7.57 (t, J=8.1 Hz, 1H), 7.34-7.27 (m, 3H), 5.70 (s, 2H), 3.96 (d, J=14.6 Hz, 1H), 3.86 (d, J=14.6 Hz, 1H), 3.79 (dd, J=4.1, 12.2 Hz, 1H), 3.50 (dd, J=5.0, 12.2 Hz, 1H), 3.37 (d, J=17.5 Hz, 1H), 3.27 (d, J=17.5 Hz, 1H), 3.07-3.02 (m, 1H), 1.73-1.66 (m, 1H), 1.61-1.54 (m, 1H), 1.41-1.31 (m, 4H), 0.94 (t, J=7.3 Hz, 3H). ¹³C NMR (CD₃OD 125 MHZ) § 169.9, 151.5 (q, J=3.4 Hz), 145.4, 139.6, 138.4, 134.4, 133.9, 133.2, 133.0, 132.6, 132.3, 126.5, 122.3, 121.7 (q, J=256.1 Hz), 121.6, 121.1, 119.9, 115.3, 58.5, 54.3, 54.0, 51.8, 47.5, 30.6, 28.4, 24.6, 15.1. ¹⁹F{¹H} NMR (CD₃OD, 470 MHz) δ −59.5. HRMS (ESI-TOF): Calcd for (C₂₇H₂₉F₃N₅O₂)⁺ 512.2268; found: 512.2285.

The intermediate compound 11g was prepared as follows.

### a. Synthesis of 3-((5-Formyl-1H-imidazol-1-yl)methyl)benzonitrile (11g, from 13; Millet, R, et al., J. Med. Chem., 2004, 47, 6812-6820)

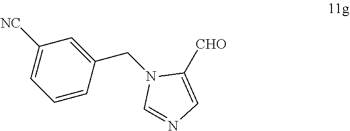 [SMILES: N#Cc1cccc(Cn2cncc2C=O)c1]

Compound 11g was prepared via the synthetic procedure for compound 11a (from 13) described above, except that compound 9g was used instead of compound 9a. Yield: 68% (215 mg). White solid. ¹H NMR (CDCl₃, 500 MHZ) δ 9.75 (s, 1H), 7.88 (s, 1H), 7.77 (s, 1H), 7.60 (d, J=7.6 Hz, 1H), 7.49-7.42 (m, 3H), 5.56 (s, 2H).

### Example 8. Synthesis(S)-5-((5-((2-Butyl-5-oxo-4-(3-(trifluoromethoxy)phenyl) piperazin-1-yl)methyl)-1H-imidazol-1-yl)methyl)-2-fluorobenzonitrile (2p)

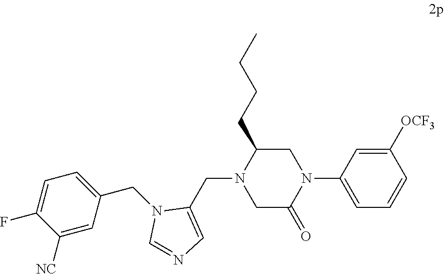 [SMILES: CCCC[C@H]1CN(c2cccc(OC(F)(F)F)c2)C(=O)CN1Cc1cncn1Cc1ccc(F)c(C#N)c1]

Compound 2p was prepared via the synthetic procedure described for compound 2f, except compound 11h was used instead of compound 11a. Yield: 53% (70 mg on a 0.25 mmol reaction scale). Colorless oil. Purity: 98% by HPLC. ¹H NMR (CD₃OD, 500 MHz) δ 9.1 (s, 1H), 7.82 (dd, J=2.4, 5.9 Hz, 1H), 7.73-7.70 (m, 2H), 7.57 (t, J=8.1 Hz, 1H), 7.48 (t, J=8.9 Hz, 1H), 7.35-7.27 (m, 3H), 5.66 (s, 2H), 3.98 (d, J=14.7 Hz, 1H), 3.88 (d, J=14.8 Hz, 1H), 3.85 (dd, J=4.2, 12.2 Hz, 1H), 3.53 (dd, J=5.0, 12.3 Hz, 1H), 3.38 (d, J=17.5 Hz, 1H), 3.28 (d, J=17.5 Hz, 1H), 3.08-3.05 (m, 1H), 1.65-1.69 (m, 1H), 1.54-1.56 (m, 1H), 1.45-1.30 (m, 4H), 0.95 (t, J=7.0 Hz, 3H). ¹³C NMR (CD₃OD 125 MHZ) δ 169.9, 165.2 (d, J=254.8 Hz), 151.5 (q, J=3.2 Hz), 145.3, 139.5, 136.9 (d, J=8.5 Hz), 135.1, 134.3 (d, J=3.7 Hz), 133.1, 132.6, 126.5, 122.7 (q, J=253.8 Hz), 122.3, 121.7, 121.0, 119.3 (d, J=20.2 Hz), 115.0, 104.0 (d, J=16.2 Hz), 58.5, 54.2, 54.0, 51.2, 47.5, 30.6, 28.4, 24.6, 15.1. ¹⁹F{¹H} NMR (CD₃OD, 470 MHz) δ −59.5, −109.5. HRMS (ESI-TOF): Calcd for (C₂₇H₂₈F₄N₅O₂)⁺ 530.2174; found: 530.2177.

The intermediate compound 11h was prepared as follows.

### a. Synthesis of 2-Fluoro-5-((5-Formyl-1H-imidazol-1-yl)methyl)benzonitrile (11h, from 13; Bergman, J. M, et al., Bioorg. Med. Chem., 2001, 11, 1411-1415)

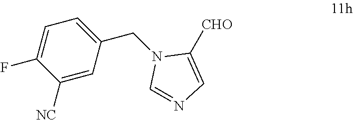 [SMILES: N#Cc1cc(Cn2cncc2C=O)ccc1F]

Compound 11h was prepared via the synthetic procedure for compound 11a (from 13) described above, except that compound 9h (Xu, X, et al., Eur. J. Med. Chem., 2018, 143, 1325-1344) was used instead of compound 9a. Yield: 72% (247 mg). Pale yellow solid. ¹H NMR (CDCl₃, 500 MHZ) δ 9.74 (s, 1H), 7.89 (s, 1H), 7.79 (s, 1H), 7.61 (dd, J=6.5, 8.1 Hz, 1H), 7.05 (dd, J=1.5, 8.0 Hz, 1H), 6.98 (dd, J=1.2, 8.5 Hz, 1H), 5.57 (s, 2H).

### Example 9. Synthesis of(S)-4-((5-((2-Butyl-5-oxo-4-(3-(trifluoromethoxy)-phenyl)piperazin-1-yl)methyl)-1H-imidazol-1-yl)methyl)-2-fluorobenzonitrile (2q)

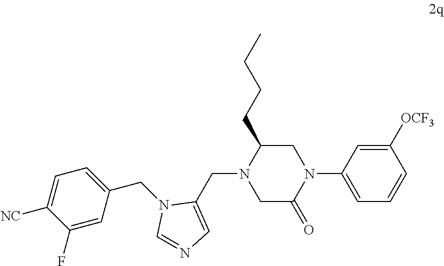 [SMILES: CCCC[C@H]1CN(c2cccc(OC(F)(F)F)c2)C(=O)CN1Cc1cncn1Cc1ccc(C#N)c(F)c1]

Compound 2q was prepared according to the synthetic procedure for compound 2f described above, except that compound 11i was used instead of compound 11a. Yield: 60% (80 mg on a 0.25 mmol reaction scale). Colorless oil. Purity: 99% by HPLC. ¹H NMR (CD₃OD, 500 MHz) δ 9.2 (s, 1H), 7.85 (t, J=7.9 Hz, 1H), 7.74 (s, 1H), 7.57 (t, J=8.2 Hz, 1H), 7.37 (dd, J=1.5, 9.9 Hz, 1H), 7.30-7.27 (m, 4H), 5.75 (s, 2H), 3.96 (d, J=14.6 Hz, 1H), 3.85 (d, J=14.6 Hz, 1H), 3.75 (dd, J=4.1, 12.2 Hz, 1H), 3.47 (dd, J=5.0, 12.2 Hz, 1H), 3.34 (d, J=17.4 Hz, 1H), 3.24 (d, J=17.4 Hz, 1H), 3.06-3.01 (m, 1H), 1.72-1.65 (m, 1H), 1.60-1.52 (m, 1H), 1.43-1.29 (m, 4H), 0.94 (t, J=7.0 Hz, 3H). ¹³C NMR (CD₃OD 125 MHz) δ 169.8, 166.5 (d, J=258.3 Hz), 151.5 (q, J=3.2 Hz), 145.5 (d, J=8.1 Hz), 145.3, 139.9, 136.6, 132.6, 126.4, 125.7 (d, J=3.5 Hz), 122.7 (q, J=256.8 Hz), 122.4, 121.6, 120.1, 117.1 (d, J=21.2 Hz), 115.0, 103.2 (d, J=15.4 Hz), 58.6, 54.2, 54.0, 51.7, 47.5, 30.6, 28.3, 24.6, 15.1. ¹⁹F{¹H} NMR (CD₃OD, 470 MHz) δ −59.5, −108.1. HRMS (ESI-TOF): Calcd for (C₂₇H₂₈F₄N₅O₂)⁺ 530.2174; found: 530.2193. MHz) δ 8.89 (s, 1H), 7.66 (s, 1H), 7.61 (d, J=8.3 Hz, 2H), 7.24 (d, J=8.2 Hz, 2H), 5.49 (s, 2H), 5.14 (s, 2H), 1.90 (s, 3H). HRMS (ESI-TOF): Calcd for (C₁₃H₁₄BrN₂O₂)⁺309.0233; found: 309.0237.

The intermediate compound 11i was prepared as follows.

### a. Synthesis of 2-Fluoro-4-((5-formyl-1H-imidazol-1-yl)methyl)benzonitrile (11i, from 13; Bergman, J. M, et al., Bioorg. Med. Chem. 2001, 11, 1411-1415)

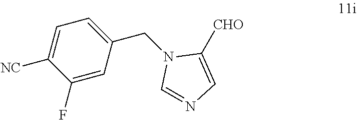 [SMILES: N#Cc1ccc(Cn2cncc2C=O)cc1F]

Compound 11i was prepared via the synthetic procedure for compound 11a (from 13) described above, except that compound 9i was used instead of compound 9a. Yield: 70% (240 mg). Pale yellow solid. ¹H NMR (CD₃OD, 500 MHz) δ 9.84 (s, 1H), 8.31 (s, 1H), 8.08 (s, 1H), 7.85 (dd, J=6.8, 8.0 Hz, 1H), 7.32 (d, J=10.0 Hz, 1H), 7.29 (dd, J=8.1 Hz, 1H), 5.79 (s, 2H).

### Examples 10-12

Synthetic route for the preparation of 2a-c.

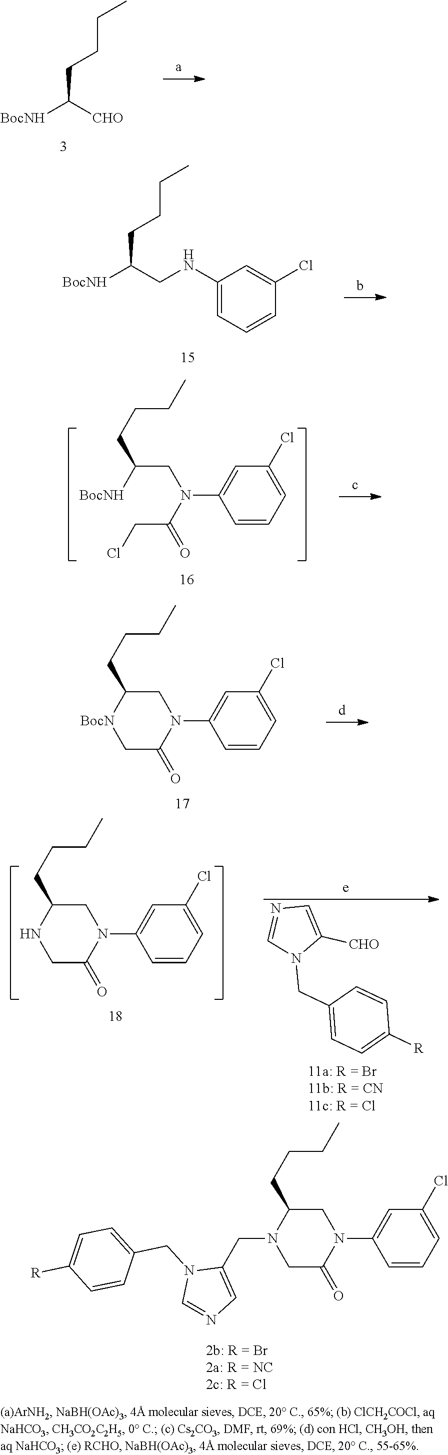

### Example 10. Synthesis of(S)-4-((5-((2-Butyl-4-(3-chlorophenyl)-5-oxopiperazin-1-yl)methyl)-1H-imidazol-1-yl)methyl)benzonitrile (2a)

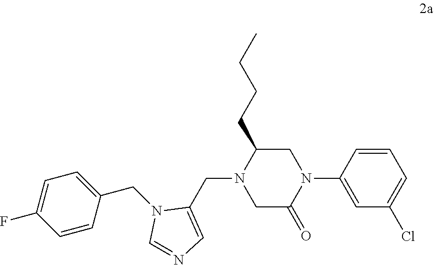 [SMILES: CCCC[C@H]1CN(c2cccc(Cl)c2)C(=O)CN1Cc1cncn1Cc1ccc(F)cc1]

Compound 2a was prepared via the synthetic procedure for compound 2f described above except using compound 11b instead of compound 11a and compound 17 in lieu of compound 6. Yield: 55% (51 mg on a 0.2 mmol reaction scale). Colorless oil. Purity: 98% by HPLC. ¹H NMR (CD₃OD, 500 MHz) δ 9.15 (s, 1H), 7.83 (d, J=8.3 Hz, 2H), 7.73 (s, 1H), 7.47-7.44 (m, 3H), 7.37-7.34 (m, 2H), 7.20 (d, J=7.8 Hz, 1H), 5.74 (s, 2H), 3.93 (d, J=14.7 Hz, 1H), 3.83 (d, J=14.7 Hz, 1H), 3.68 (dd, J=4.2, 12.2 Hz, 1H), 3.41 (dd, J=5.0, 12.2 Hz, 1H), 3.33 (d, J=16.4 Hz, 1H), 3.22 (d, J=17.1 Hz, 1H), 3.03-2.98 (m, 1H), 1.70-1.64 (m, 1H), 1.58-1.50 (m, 1H), 1.41-1.27 (m, 4H), 0.93 (t, J=7.3 Hz, 3H). ¹³C{¹H} NMR (CD₃OD 125 MHZ) δ 169.8, 145.1, 142.2, 139.8, 136.5, 135.0, 133.3, 132.5, 129.8, 129.4, 128.3, 126.2, 122.3, 119.9, 114.5, 58.5, 54.2, 54.1, 52.1, 47.5, 30.6, 28.2, 24.6, 15.1. HRMS (ESI-TOF): Calcd for (C₂₆H₂₉ClN₅O)⁺462.2055; found: 462.2059.

The intermediate compound 17 was prepared as follows.

### a. Synthesis of tert-Butyl(S)-(1-((3-chlorophenyl)amino) hexan-2-yl) carbamate (15)

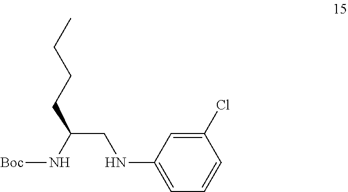 [SMILES: CCCC[C@@H](CNc1cccc(Cl)c1)NC(=O)OC(C)(C)C]

Compound 15 was prepared using the procedure noted for compound 4 described above, except that 3-chloroaniline was used instead of 3-(trifluoromethoxy) aniline. Yield: 65% (744 mg on a 3.5 mmol reaction scale). Pale yellow oil. ¹H NMR (CDCl₃, 500 MHZ) δ 7.05 (t, J=8.0 Hz, 1H), 6.63 (d, J=7.8 Hz, 1H), 6.54 (s, 1H), 6.46 (t, J=8.2 Hz, 1H), 4.45 (bs, 2H), 3.80 (bs, 1H), 3.20-3.17 (m, 1H), 3.01-2.97 (m, 1H), 1.57-1.54 (m, 1H), 1.45 (s, 9H), 1.39-1.31 (m, 5H), 0.91 (t, J=6.6 Hz, 3H). ¹³C{¹H} NMR (CDCl₃, 125 MHZ) δ 156.5, 149.6, 135.0, 130.1, 116.8, 112.0, 111.0, 79.7, 50.6, 49.4, 33.0, 28.4, 28.2, 22.6, 13.9. HRMS (ESI-TOF): Calcd for (C₁₇H₂₈ClN₂O₂)⁺ 327.1834; found: 327.1853.

### b. Synthesis of tert-Butyl(S)-2-butyl-4-(3-chlorophenyl)-5-oxopiperazine-1-carboxylate (17)

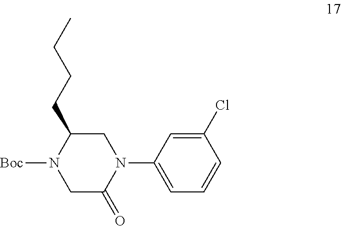 [SMILES: CCCC[C@H]1CN(c2cccc(Cl)c2)C(=O)CN1C(=O)OC(C)(C)C]

Compound 17 was prepared via the synthetic procedure for compound 6 described above by using compound 15 instead of compound 4. Yield: 69% (537 mg on a 2 mmol reaction scale). Pale yellow oil. ¹H NMR (CDCl₃, 500 MHZ) δ 7.34 (t, J=7.9 Hz, 1H), 7.29-7.26 (m, 2H), 7.17 (d, J=8.0 Hz, 1H), 4.43 (bs, 2H), 4.01 (dd, J=4.0, 12.3 Hz, 1H), 3.89 (d, J=18.8 Hz, 1H), 3.42 (d, J=12.3 Hz, 1H), 1.85-1.77 (m, 1H), 1.65-1.58 (m, 1H), 1.50 (s, 9H), 1.44-1.25 (m, 4H), 0.92 (t, J=6.9 Hz, 3H). ¹³C{¹H} NMR (CDCl₃, 125 MHz) δ 165.7, 153.8, 143.2, 134.9, 130.4, 127.5, 126.1, 123.9, 80.9, 53.3, 31.1, 29.6, 28.5, 28.3, 22.5, 14.1. HRMS (ESI-TOF): Calcd for (C₁₉H₂₇ClN₂NaO₃)⁺ 389.1602; found: 389.1617.

### Example 11. Synthesis of(S)-4-((1-(4-Bromobenzyl)-1H-imidazol-5-yl)methyl)-5-butyl-1-(3-chlorophenyl)piperazin-2-one (2b)

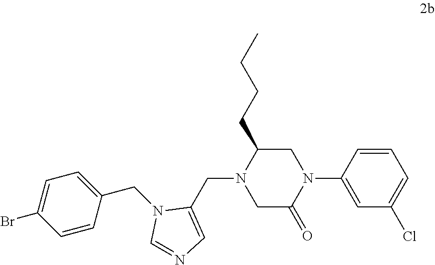 [SMILES: CCCC[C@H]1CN(c2cccc(Cl)c2)C(=O)CN1Cc1cncn1Cc1ccc(Br)cc1]

Compound 2b was prepared via the synthetic procedure for compound 2f described above except that compound 17 was used in lieu of compound 6. Yield: 51% (58 mg in a 0.22 mmol reaction scale). Colorless oil. Purity: 99% by HPLC. ¹H NMR (CD₃OD, 500 MHz) δ 9.09 (s, 1H), 7.70 (s, 1H), 7.63 (d, J=8.4 Hz, 2H), 7.46 (t, J=7.9 Hz, 1H), 7.37 (s, 1H), 7.36 (d, J=7.8 Hz, 1H), 7.24-7.21 (m, 3H), 5.60 (s, 2H), 3.93 (d, J=14.6 Hz, 1H), 3.83 (d, J=14.6 Hz, 1H), 3.69 (dd, J=4.2, 12.2 Hz, 1H), 3.42 (dd, J=4.9, 12.2 Hz, 1H), 3.36 (d, J=14.9 Hz, 1H), 3.26 (d, J=17.5 Hz, 1H), 3.03-2.99 (m, 1H), 1.71-1.66 (m, 1H), 1.60-1.52 (m, 1H), 1.41-1.28 (m, 4H), 0.94 (t, J=6.9 Hz, 3H). ¹³C{¹H} NMR (CD₃OD 125 MHZ) δ 169.9, 145.1, 139.4, 136.5, 135.9, 134.3, 133.3, 132.5, 131.0, 129.4, 128.4, 126.3, 124.6, 122.1, 58.4, 54.2, 52.0, 47.5, 30.6, 28.1, 24.6, 15.1. HRMS (ESI-TOF): Calcd for (C₂₅H₂₉BrClN₄O)⁺515.1208; found: 515.1203.

### Example 12. Synthesis of(S)-5-Butyl-4-((1-(4-chlorobenzyl)-1H-imidazol-5-yl)methyl)-1-(3-chlorophenyl)piperazin-2-one (2c)

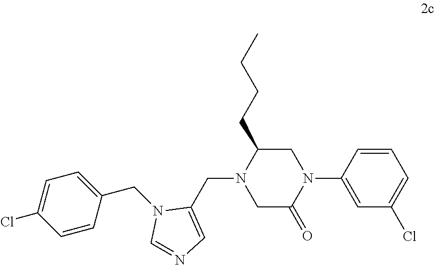 [SMILES: CCCC[C@H]1CN(c2cccc(Cl)c2)C(=O)CN1Cc1cncn1Cc1ccc(Cl)cc1]

Compound 2c was prepared via the synthetic procedure for compound 2f described above using compound 11c instead of compound 11a and compound 17 in lieu of compound 6. Yield: 46% (50 mg in a 0.23 mmol reaction scale). Colorless oil. Purity: 98% by HPLC. ¹H NMR (CD₃OD, 500 MHz) δ 9.08 (s, 1H), 7.69 (s, 1H), 7.47 (d, J=8.2 Hz, 2H), 7.44 (t, J=8.0 Hz, 1H), 7.36 (s, 1H), 7.35 (d, J=7.8 Hz, 1H), 7.29 (d, J=8.1 Hz, 2H), 7.21 (d, J=7.9 Hz, 1H), 5.61 (s, 2H), 3.93 (d, J=14.6 Hz, 1H), 3.84 (d, J=14.6 Hz, 1H), 3.70 (dd, J=4.0, 12.6 Hz, 1H), 3.43 (dd, J=4.7, 12.2 Hz, 1H), 3.36 (d, J=17.5 Hz, 1H), 3.26 (d, J=17.5 Hz, 1H), 3.04-2.98 (m, 1H), 1.73-1.66 (m, 1H), 1.60-1.52 (m, 1H), 1.42-1.28 (m, 4H), 0.93 (t, J=7.2 Hz, 3H). ¹³C{¹H} NMR (CD₃OD 125 MHz) δ 169.9, 145.1, 139.4, 136.6, 136.5, 135.4, 133.2, 132.5, 131.3, 130.8, 129.4, 128.4, 126.3, 122.1, 58.4, 54.2, 52.0, 47.5, 30.6, 28.1, 24.6, 15.1. HRMS (ESI-TOF): Calcd for (C₂₅H₂₉Cl₂N₄O)⁺ 471.1713; found: 471.1719.

### Examples 13-15

Synthetic route for the preparation of 2j and 2r-s.

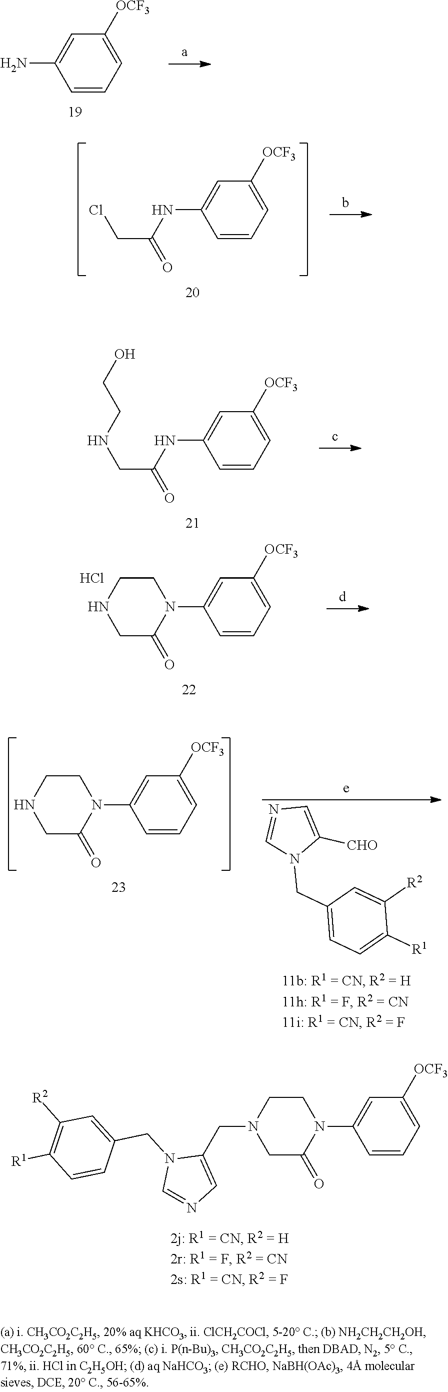

### Example 13. Synthesis of (S)-4-((5-((3-Oxo-4-(3-(trifluoromethoxy)phenyl) piperazin-1-yl)methyl)-1H-imidazol-1-yl)methyl)benzonitrile (2j)

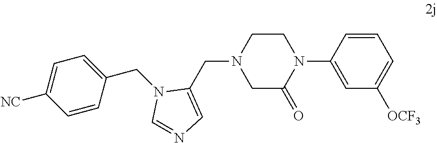 [SMILES: N#Cc1ccc(Cn2cncc2CN2CCN(c3cccc(OC(F)(F)F)c3)C(=O)C2)cc1]

Compound 22 (62.4 mg, 0.24 mmol) was added into saturated aqueous NaHCO₃ solution (50 mL), and extracted with EtOAc (3×50 mL), the combined extracts were dried over anhydrous MgSO₄, filtered, and concentrated under reduced pressure. To the crude product in ClCH₂CH₂Cl (3 mL) above was added 11b (42 mg, 0.2 mmol), NaBH(OAc)₃ (106 mg, 0.5 mmol), 4 Å sieves (broken, 200 mg), and AcOH (1 drop), and the mixture was kept at rt for 1 day, the reaction was quenched by the addition of aqueous NaHCO₃ solution, and filtered through celite to remove 4 Å sieves. The filtrate was poured into H₂O, and extracted with EtOAc (3×50 mL), the combined extracts were dried over anhydrous MgSO₄, filtered, and concentrated under reduced pressure. The final product was purified using preparative HPLC (Solvent A: H₂O—0.1% TFA, Solvent B: ACN—0.1% TFA, Solvent gradient: 0% B—5 min followed by 0-100% B over 60 min) to afford 60 mg pure product (66% yield) as colorless oil. Purity: 99% by HPLC. ¹H NMR (CD₃OD, 500 MHz) δ 9.2 (s, 1H), 7.81 (d, J=8.3 Hz, 2H), 7.71 (s, 1H), 7.56 (t, J=8.0 Hz, 1H), 7.49 (d, J=8.4 Hz, 2H), 7.29-7.26 (m, 3H), 5.74 (s, 2H), 3.71 (s, 2H), 3.52 (t, J=5.2 Hz, 2H), 3.23 (s, 2H), 2.84 (t, J=5.3 Hz, 2H). ¹³C {¹H} NMR (CD₃OD 125 MHz) δ 169.3, 151.5 (q, J=3.3 Hz), 145.1, 142.5, 139.8, 134.9, 132.5, 130.0, 126.4, 122.7 (q, J=256.1 Hz) 122.5, 121.5, 121.0, 120.0, 114.4, 58.4, 52.1, 51.6, 50.9, 50.6. ¹⁹F{¹H} NMR (CD₃OD, 470 MHz) δ −59.5. HRMS (ESI-TOF): Calcd for (C₂₃H₂₁F₃N₅O₂)⁺ 456.1642; found: 456.1637.

The intermediate compound 22 was prepared as follows.

### a. Synthesis of 2-((2-Hydroxyethyl)amino)-N-(3-(trifluoromethoxy)phenyl) acetamide (21; Weissman, S. A, et al., Tet. Lett., 1998, 39, 7459-7462)

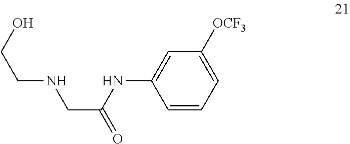 [SMILES: O=C(CNCCO)Nc1cccc(OC(F)(F)F)c1]

Aniline (19.8 mmol, 3.5 g) was combined with EtOAc (25 mL) and 20% aq KHCO₃ (25 mL). The biphasic mixture was cooled to 5° C. and treated with chloroacetyl chloride (25.1 mmol, 2.84 g, 2.0 mL) dropwise over 30 min. The reaction mixture was warmed to 25° C. and the aqueous layer was removed. The organic layer was combined with ethanolamine (70.5 mmol, 4.3 g, 4.3 mL), heated to 60° C. and stirred for 2 h. Water (6 mL) and EtOAC (5 mL) were added to the mixture. The solution was heated again to 60° C. and the aqueous layer removed. The organic layer was cooled to 0° C. over 1 h and stirred for 1 h. The solids were collected and washed with EtOAC (3×25 mL). The product was dried in vacuo at 40° C. to a constant weight to provide the desired intermediate (13 mmol, 3.6 g). Yield: 65%. White solid. ¹H NMR (CD₃OD, 500 MHz) δ 6.24 (s, 1H), 5.98 (d, J=8.5 Hz, 1H), 5.87 (t, J=8.2 Hz, 1H), 5.47 (d, J=8.2 Hz, 1H), 2.12 (t, J=5.2 Hz, 2H), 1.91 (s, 2H), 1.25 (t, J=5.3 Hz, 2H). ¹³C{¹H} NMR (CD₃OD 125 MHz) δ 173.6, 151.6 (q, J=3.0 Hz), 142.1, 132.0, 123.7 (q, J=255.9 Hz), 120.0, 117.9, 114.3, 62.8, 54.2, 53.4. ¹⁹F{¹H} NMR (CD₃OD, 470 MHz) δ −55.5. HRMS (ESI-TOF): Calcd for (C₁₁H₁₃F₃N₂NaO₃)⁺ 301.0770; found: 301.0770.

### b. Synthesis of 1-(3-(Trifluoromethoxy)phenyl) piperazin-2-one (22; Weissman, S. A, et al., Tet. Lett., 1998, 39, 7459-7462)

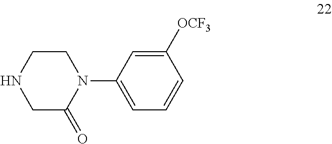 [SMILES: O=C1CNCCN1c1cccc(OC(F)(F)F)c1]

Aminoalcohol 21 (18 mmol, 5 g) was combined with EtOAc (32 mL) and tributylphosphine (26 mmol, 5.2 g, 6.4 mL) under N₂ at 0° C. A di-butylazodicarboxamide solution in EtOAc (20 mL) (25.6 mmol, 5.9 g) was added dropwise over 20 min. The reaction mixture was kept at 5° C. for 15 min and then warmed to 40° C. Ethanolic HCl (5 mL; 4.2 N solution) was added. The resulting slurry was cooled to 5° C. and kept at this temperature for 4 h. The product (12.8 mmol, 3.78 g) was isolated by filtration and washed by EtOAc. Yield: 71%. White solid. ¹H NMR (CD₃OD, 500 MHz) δ 6.15 (t, J=8.1 Hz, 1H), 6.01 (d, J=8.0 Hz, 1H), 5.99 (s, 1H), 5.88 (d, J=8.4 Hz, 1H), 2.63 (s, 2H), 2.61 (t, J=5.3 Hz, 2H), 2.29 (t, J=5.9 Hz, 2H). ¹³C{¹H} NMR (CD₃OD 125 MHZ) δ 164.5, 151.5 (q, J=3.0 Hz), 144.5, 132.8, 126.8, 122.7 (q, J=256.1 Hz), 122.2, 121.3, 48.6, 47.3, 42.8. ¹⁹F{¹H} NMR (CD₃OD, 470 MHz) δ −59.5. HRMS (ESI-TOF): Calcd for (C₁₁H₁₂F₃N₂O₂)⁺ 261.0846; found: 261.0862.

### Example 14. Synthesis of 2-Fluoro-5-((5-((3-oxo-4-(3-(trifluoromethoxy)phenyl) piperazin-1-yl)methyl)-1H-imidazol-1-yl)methyl)benzonitrile (2r)

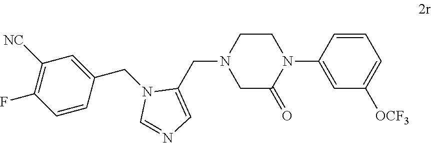 [SMILES: N#Cc1cc(Cn2cncc2CN2CCN(c3cccc(OC(F)(F)F)c3)C(=O)C2)ccc1F]

Compound 2r was prepared using the synthetic procedure for compound 2j described above except using compound 11h instead of compound 11b. Yield: 65% (77 mg on a 0.25 mmol reaction scale). Colorless oil. Purity: 99% by HPLC. ¹H NMR (CD₃OD, 500 MHZ) δ 9.1 (s, 1H), 7.86 (dd, J=2.2, 5.9 Hz, 1H), 7.78-7.74 (m, 1H), 7.71 (s, 1H), 7.56 (t, J=8.1 Hz, 1H), 7.47 (t, J=8.8 Hz, 1H), 7.34-7.27 (m, 3H), 5.66 (s, 2H), 3.75 (s, 2H), 3.64 (t, J=5.2 Hz, 2H), 3.27 (s, 2H), 2.90 (t, J=5.4 Hz, 2H). ¹³C NMR (CD₃OD 125 MHZ) δ 169.3, 165.8 (d, J=258.7 Hz), 151.5 (q, J=3.2 Hz), 145.2, 139.6, 137.9 (d, J=8.9 Hz), 135.3, 134.3 (d, J=3.6 Hz), 132.5, 132.4, 126.5, 122.7 (q, J=256.2 Hz), 122.4, 121.6, 121.1, 119.2 (d, J=20.2 Hz), 115.1, 103.9 (d, J=16.2 Hz), 58.4, 51.7, 51.2, 50.9, 50.7. ¹⁹F{¹H} NMR (CD₃OD, 470 MHz) δ −59.5, −109.6. HRMS (ESI-TOF): Calcd for (C₂₃H₂₀F₄N₅O₂)⁺ 474.1548; found: 474.1539.

### Example 15. Synthesis of 2-Fluoro-4-((5-((3-Oxo-4-(3-(trifluoromethoxy)phenyl) piperazin-1-yl)methyl)-1H-imidazol-1-yl)methyl)benzonitrile (2s)

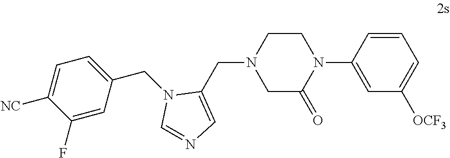 [SMILES: N#Cc1ccc(Cn2cncc2CN2CCN(c3cccc(OC(F)(F)F)c3)C(=O)C2)cc1F]

Compound 2s was prepared via the synthetic procedure for compound 2j described above except using compound 11i instead of compound 11b. Yield: 71% (77.5 mg on a 0.23 mmol reaction scale). Colorless oil. Purity: 99% by HPLC. ¹H NMR (CD₃OD, 500 MHZ) δ 9.2 (s, 1H), 7.83 (t, J=8.0 Hz, 1H), 7.73 (s, 1H), 7.56 (t, J=8.0 Hz, 1H), 7.41 (dd, J=1.7, 9.0 Hz, 1H), 7.32-7.26 (m, 4H), 5.75 (s, 2H), 3.75 (s, 2H), 3.56 (t, J=5.2 Hz, 2H), 3.23 (s, 2H), 2.87 (t, J=5.5 Hz, 2H). 13C NMR (CD₃OD 125 MHz) δ 169.2, 166.5 (d, J=257.2 Hz), 151.5 (q, J=3.2 Hz), 145.6 (d, J=8.1 Hz), 145.1, 139.9, 136.4, 132.6, 126.4, 125.9 (d, J=3.5 Hz), 122.7 (q, J=256.2 Hz), 122.4, 121.6, 121.0, 117.4 (d, J=21.4 Hz), 115.0, 103.2 (d, J=15.4 Hz), 58.3, 51.7, 51.6, 50.9, 50.7. ¹⁹F{¹H} NMR (CD₃OD, 470 MHz) δ −59.5, −108.1. HRMS (ESI-TOF): Calcd for (C₂₃H₂₀F₄N₅O₂)⁺ 474.1548; found: 474.1535.

### Examples 16-17

Synthetic route for the preparation of 2k-l.

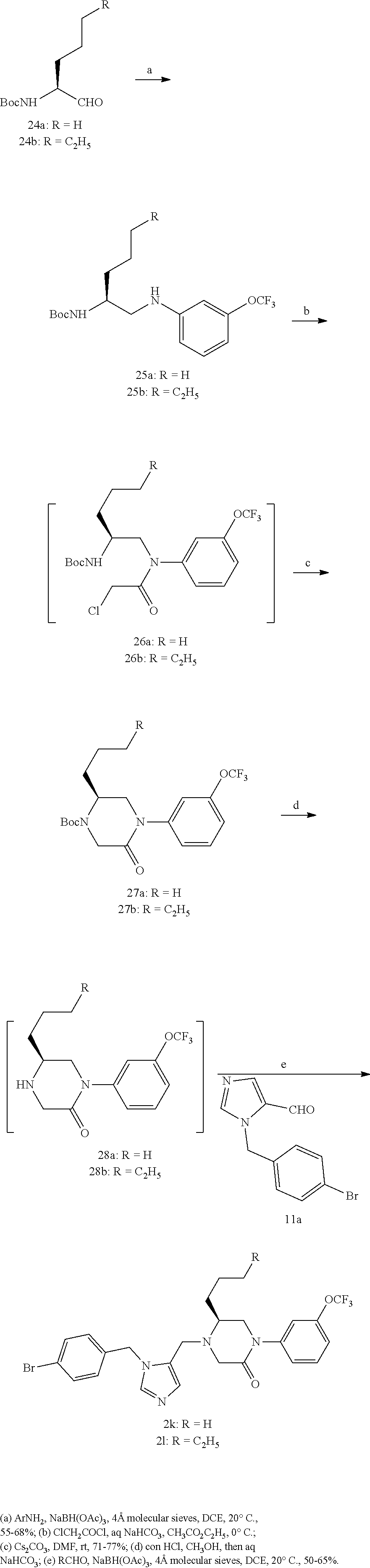

### Example 16. Synthesis of(S)-4-((1-(4-Bromobenzyl)-1H-imidazol-5-yl)methyl)-5-propyl-1-(3-(trifluoromethoxy)phenyl) piperazin-2-one (2k)

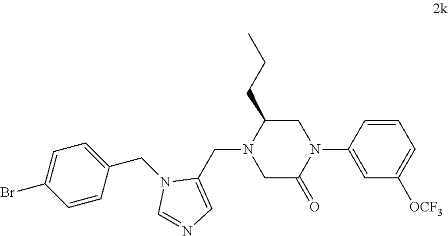 [SMILES: CCC[C@H]1CN(c2cccc(OC(F)(F)F)c2)C(=O)CN1Cc1cncn1Cc1ccc(Br)cc1]

Compound 2k was prepared using the synthetic procedure for compound 2f described above except that compound 27a was used instead of compound 6. Yield: 61% (67 mg on a 0.2 mmol reaction scale). Colorless oil. Purity: 99% by HPLC. ¹H NMR (CD₃OD, 500 MHZ) δ 9.09 (s, 1H), 7.70 (s, 1H), 7.62 (d, J=8.5 Hz, 2H), 7.57 (t, J=8.5 Hz, 1H), 7.31-7.23 (m, 5H), 5.60 (s, 2H), 3.94 (d, J=14.6 Hz, 1H), 3.84 (d, J=14.6 Hz, 1H), 3.73 (dd, J=4.2, 12.2 Hz, 1H), 3.46 (dd, J=4.9, 12.2 Hz, 1H), 3.37 (d, J=17.5 Hz, 1H), 3.27 (d, J=17.5 Hz, 1H), 3.06-3.01 (m, 1H), 1.71-1.64 (m, 1H), 1.59-1.51 (m, 1H), 1.43-1.31 (m, 2H), 0.97 (dd, J=7.4 Hz, 3H). ¹³C{¹H} NMR (CD₃OD 125 MHZ) δ 169.9, 151.5 (q, J=3.2 Hz), 145.4, 139.4, 135.9, 134.3, 133.2, 132.6, 131.1, 126.6, 124.6, 122.7 (q, J=254.2 Hz), 122.1, 121.5, 121.1, 58.1, 54.3, 54.1, 52.0, 47.5, 30.6, 21.6, 15.3. ¹⁹F{¹H} NMR (CD₃OD, 470 MHZ) δ −59.5. HRMS (ESI-TOF): Calcd for (C₂₅H₂₇BrF₃N₄O₂)⁺ 551.1264; found: 551.1279.

The intermediate compound 27a was prepared as follows.

### a. Synthesis of tert-Butyl(S)-(1-((3-(trifluoromethoxy)phenyl)amino)pentan-2-yl)carbamate (25a)

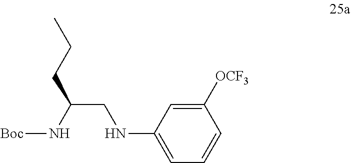 [SMILES: CCC[C@@H](CNc1cccc(OC(F)(F)F)c1)NC(=O)OC(C)(C)C]

Compound 25a was prepared via the synthetic procedure for compound 4 described above, except that 24a was used instead of compound 3. Yield: 68% (785 mg on a 3 mmol reaction scale). Pale yellow oil. ¹H NMR (CDCl₃, 500 MHZ) δ 7.14 (t, J=8.1 Hz, 1H), 6.54-6.50 (m, 2H), 6.41 (s, 1H), 4.49 (bs, 2H), 3.86 (bs, 1H), 3.24-3.20 (m, 1H), 3.05-3.00 (m, 1H), 1.56-1.41 (m, 4H), 1.47 (s, 9H), 0.97 (t, J=6.9 Hz, 3H). ¹³C{¹H} NMR (CDCl₃, 125 MHZ) δ 156.6, 150.5, 149.8 (q, J=3.0 Hz), 130.1, 120.5 (q, J=256.8 Hz), 110.9, 108.8, 104.5, 79.7, 50.3, 49.5, 35.4, 28.3, 19.3, 13.9. ¹⁹F{¹H} NMR (CDCl₃, 470 MHz) δ −57.5. HRMS (ESI-TOF): Calcd for (C₁₇H₂₅F₃N₂NaO₃)⁺385.1709; found: 385.1686.

### b. Synthesis of tert-Butyl(S)-5-oxo-2-propyl-4-(3-(trifluoromethoxy)phenyl)piperazine-1-carboxylate (27a)

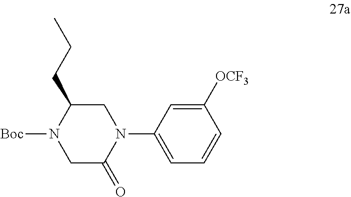 [SMILES: CCC[C@H]1CN(c2cccc(OC(F)(F)F)c2)C(=O)CN1C(=O)OC(C)(C)C]

Compound 27a was prepared via the synthetic procedure for compound 6 described previously by using compound 25a instead of compound 4. Yield: 71% (453 mg on a 1.5 mmol reaction scale). Pale yellow oil. ¹H NMR (CDCl₃, 500 MHz) δ 7.45 (t, J=8.1 Hz, 1H), 7.26 (d, J=8.1 Hz, 1H), 7.18-7.16 (m, 2H), 4.49 (bs, 2H), 4.07 (dd, J=3.9, 12.2 Hz, 1H), 3.92 (d, J=18.8 Hz, 1H), 3.46 (d, J=12.2 Hz, 1H), 1.85-1.81 (m, 1H), 1.64-1.58 (m, 1H), 1.52 (s, 9H), 1.42-1.36 (m, 2H), 0.99 (t, J=7.3 Hz, 3H). ¹³C{¹H} NMR (CDCl₃, 125 MHZ) δ 165.5, 153.8, 149.5 (q, J=3.0 Hz), 143.3, 130.3, 123.8, 120.4 (q, J=257.8 Hz), 119.3, 118.3, 80.9, 53.1, 31.9, 30.9, 28.3, 19.2, 13.8. ¹⁹F{¹H} NMR (CDCl₃, 470 MHz) δ −57.8. HRMS (ESI-TOF): Calcd for (C₁₉H₂₅F₃N₂NaO₄)⁺ 425.1658; found: 425.1660.

### Example 17. Synthesis of(S)-4-((1-(4-Bromobenzyl)-1H-imidazol-5-yl)methyl)-5-pentyl-1-(3-(trifluoromethoxy)phenyl)piperazin-2-one (21)

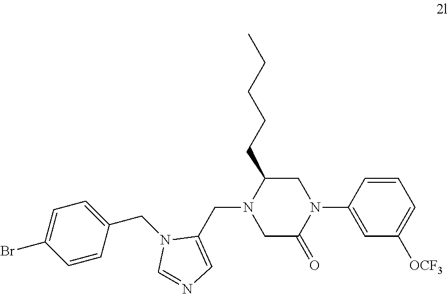 [SMILES: CCCCC[C@H]1CN(c2cccc(OC(F)(F)F)c2)C(=O)CN1Cc1cncn1Cc1ccc(Br)cc1]

Compound 21 was prepared using the synthetic procedure for compound 2f described above except that compound 27b was used instead of compound 6. Yield: 53% (64 mg on a 0.21 mmol reaction scale). Colorless oil. Purity: 98% by HPLC. ¹H NMR (CD₃OD, 500 MHZ) δ 9.09 (s, 1H), 7.70 (s, 1H), 7.63 (d, J=8.2 Hz, 2H), 7.46 (t, J=7.6 Hz, 2H), 7.31-7.27 (m, 3H), 7.23 (d, J=8.4 Hz, 1H), 5.61 (s, 2H), 3.94 (d, J=14.6 Hz, 1H), 3.84 (d, J=14.6 Hz, 1H), 3.74 (dd, J=4.1, 12.4 Hz, 1H), 3.47 (dd, J=5.0, 12.2 Hz, 1H), 3.38 (d, J=17.5 Hz, 1H), 3.28 (d, J=17.5 Hz, 1H), 3.05-3.00 (m, 1H), 1.70-1.65 (m, 1H), 1.59-1.52 (m, 1H), 1.37-1.29 (m, 6H), 0.92 (t, J=6.6 Hz, 3H). ¹³C{¹H} NMR (CD₃OD 125 MHZ) δ 169.9, 151.5 (q, J=3.0 Hz), 145.3, 139.4, 135.8, 134.3, 133.2, 132.6, 131.1, 126.6, 124.6, 122.7 (q, J=256.2 Hz), 122.1, 121.6, 121.1, 58.5, 54.3, 54.1, 52.0, 47.5, 33.8, 28.4, 28.1, 24.4, 15.2. ¹⁹F{¹H} NMR (CD₃OD, 470 MHZ) δ −59.5. HRMS (ESI-TOF): Calcd for (C₂₇H₃₁BrF₃N₄O₂)⁺ 579.1577; found: 579.1586.

The intermediate compound 27b was prepared as follows.

Synthetic procedure for the preparation of 24b used in the preparation of 21.

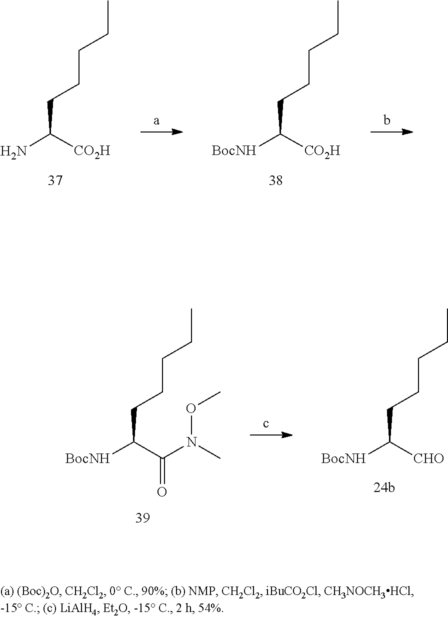

### a. Synthesis of (S)-2-((tert-butoxycarbonyl)amino)heptanoic acid (38)

Boc₂O (10.3 mmol, 2.26 g) was added to an H₂O/acetone (20/20 mL) solution of 2-aminoheptanoic acid 37 (6.9 mmol, 1 g) and Na₂CO₃ (13.8 mmol, 1.46 g). The reaction was stirred at rt for 24 h and then Et₂O was added. The layers were separated and aqueous layer was acidified to pH˜1 with an 1M aqueous solution of KHSO₄. The acid aqueous layer was washed with Et₂O (3×100 mL) and then the organic layers were combined, dried over MgSO₄, and concentrated in vacuo to give desired product 38 (1.54 g) as a colorless oil in 91% yield. ¹H NMR (CDCl₃, 500 MHz) δ 4.96 (bs, 1H), 4.28-4.35 (m, 1H), 1.93-1.83 (m, 1H), 1.73-1.63 (m, 1H), 1.48 (s, 9H), 1.41-1.31 (m, 6H), 0.91 (t, J=6.8 Hz, 3H). HRMS (ESI-TOF): Calcd for (C₁₂H₂₃NNaO₄)⁺268.1519; found: 268.1517.

### b. Synthesis of tert-Butyl(S)-(1-oxoheptan-2-yl)carbamate (24b)

2-((tert-butoxycarbonyl)amino) heptanoic acid 38 (4.08 mmol, 1g) was dissolved in a mixture of dry CH₂Cl₂ (100 mL) and N-methylmorpholine (8.16 mmol, 824 mg). This mixture was cooled to −15° C. and isobutyl chloroformate (4.08 mmol, 558 mg, 0.53 mL) was added. After stirring for 30 min at −15° C., N,O-dimethylhydroxylamine hydrochloride (4.08 mmol, 396 mg) was added to the mixture. It was stirred for an additional 1 h at −15° C. and then for 1 day at rt, followed by the addition of EtOAc (100 mL). The organic layer was extracted with 1N HCl (100 mL), saturated NaHCO₃ solution (100 mL) and brine (100 mL). After drying the organic layer over MgSO₄, the solvents were removed in vacuo. The crude product 39 was used for the next step without purification. Crude 39 from last step was dissolved in dry Et₂O (150 mL) and cooled to −20° C. LiAlH₄ (100 mmol, 380 mg) was added in portion to the solution. After stirring for 2 h, the solution was hydrolyzed with saturated KHCO₃ solution (100 mL) and any precipitated solids were removed by filtration. The aqueous layer was extracted with Et₂O (3×100 mL). The combined organic extracts were washed with 1N HCl (3×100 mL), saturated NaHCO₃ (100 mL) and brine (100 mL). The organic layer was dried over MgSO₄ and evaporated to dryness. The crude product was purified by flash chromatography on SiO₂ (Hexanes/EtOAc, 10:1, v/v) to give 24b (504 mg) as a colorless oil in 54% yield. ¹H NMR (CDCl₃, 500 MHZ) δ 9.61 (s, 1H), 5.04 (bs, 1H), 4.27-4.23 (m, 1H), 1.91-1.86 (m, 1H), 1.63-1.57 (m, 1H), 1.47 (s, 9H), 1.41-1.28 (m, 6H), 0.91 (t, J=6.8 Hz, 3H). HRMS (ESI-TOF): Calcd for (C₁₂H₂₃NNaO₃)⁺ 252.1570; found: 252.1559.

### c. Synthesis of tert-Butyl(S)-(1-((3-(trifluoromethoxy)phenyl)amino)heptan-2-yl)carbamate (25b)

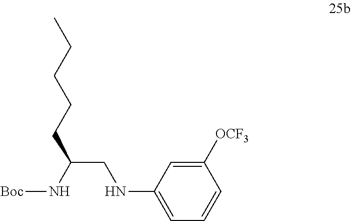 [SMILES: CCCCC[C@@H](CNc1cccc(OC(F)(F)F)c1)NC(=O)OC(C)(C)C]

Compound 25b was prepared via the synthetic procedure for compound 4 described previously, except that 24b was used instead of compound 3. Yield: 55% (860 mg on a 4 mmol reaction scale). Pale yellow oil. ¹H NMR (CDCl₃, 500 MHz) δ 7.15 (t, J=8.1 Hz, 1H), 6.54-6.50 (m, 2H), 6.41 (s, 1H), 4.45 (bs, 2H), 3.83 (bs, 1H), 3.24-3.21 (m, 1H), 3.00-2.99 (m, 1H), 1.60-1.31 (m, 8H), 1.47 (s, 9H), 0.92 (t, J=6.9 Hz, 3H). ¹³C NMR (CDCl₃, 125 MHZ) δ 156.6, 150.5, 149.7 (q, J=3.0 Hz), 130.1, 120.5 (q, J=256.8 Hz), 110.9, 108.8, 104.5, 79.7, 50.6, 49.5, 33.2, 31.6, 28.3, 25.7, 22.5, 14.0. ¹⁹F{¹H} NMR (CDCl₃, 470 MHz) δ −57.5. HRMS (ESI-TOF): Calcd for (C₁₉H₃₀F₃N₂O₃)⁺ 391.2203; found: 391.2204.

### d. Synthesis of tert-Butyl(S)-5-oxo-2-pentyl-4-(3-(trifluoromethoxy)phenyl)piperazine-1-carboxylate (27b)

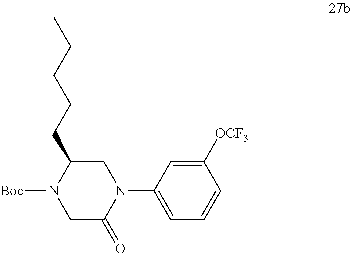 [SMILES: CCCCC[C@H]1CN(c2cccc(OC(F)(F)F)c2)C(=O)CN1C(=O)OC(C)(C)C]

Compound 27b was prepared via the synthetic procedure for compound 6 described above except that compound 25b was used instead of compound 4. Yield: 77% (523 mg on a 1.5 mmol reaction scale). Pale yellow oil. ¹H NMR (CDCl₃, 500 MHZ) δ 7.45 (t, J=8.1 Hz, 1H), 7.25 (d, J=8.8 Hz, 1H), 7.18 (s, 1H), 7.16 (d, J=8.4 Hz, 1H), 4.48 (bs, 2H), 4.06 (dd, J=4.0, 12.2 Hz, 1H), 3.92 (d, J=18.8 Hz, 1H), 3.47 (d, J=12.2 Hz, 1H), 1.82-1.80 (m, 1H), 1.63-1.59 (m, 1H), 1.51 (s, 9H), 1.36-1.28 (m, 6H), 0.90 (t, J=6.7 Hz, 3H). ¹³C NMR {¹H} (CDCl₃, 125 MHz) δ 165.6, 153.7, 149.5 (q, J=3.0 Hz), 143.3, 130.3, 123.8, 120.4 (q, J=257.9 Hz), 119.3, 118.3, 80.8, 53.0, 31.4, 30.9, 29.6, 28.3, 25.6, 22.5, 13.9. ¹⁹F{¹H} NMR (CDCl₃, 470 MHZ) δ −57.8. HRMS (ESI-TOF): Calcd for (C₂₁H₂₉F₃N₂NaO₄)⁺ 453.1972; found: 453.1969.

### Example 18

Synthetic route for the preparation of 2m.

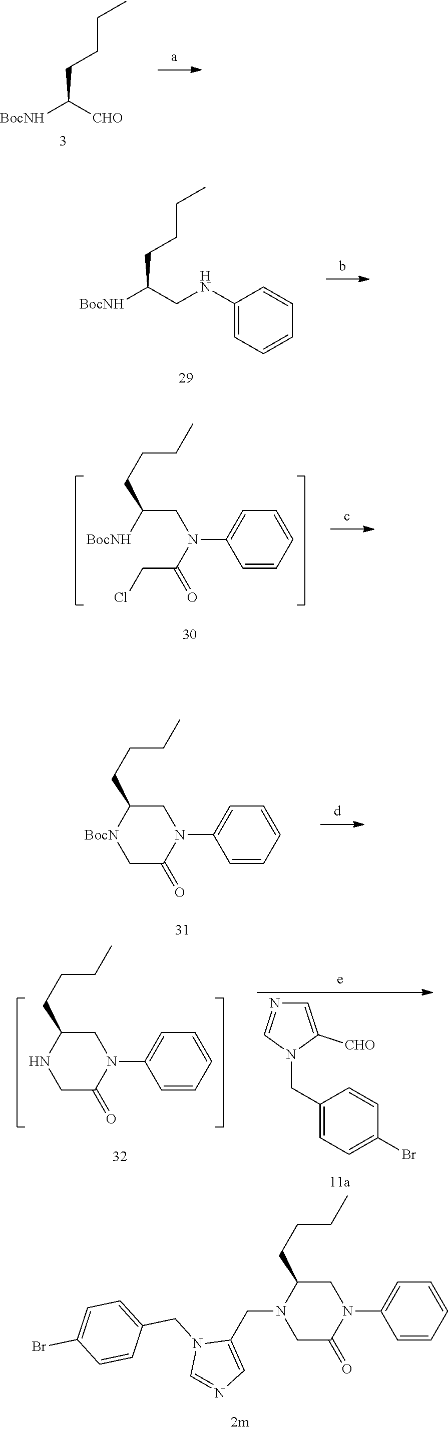

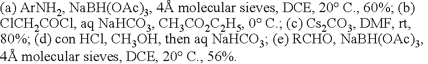

### Example 18. Synthesis of(S)-4-((1-(4-Bromobenzyl)-1H-imidazol-5-yl)methyl)-5-butyl-1-phenylpiperazin-2-one (2m)

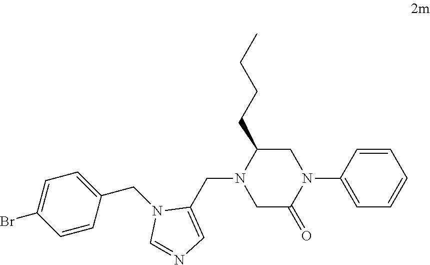 [SMILES: CCCC[C@H]1CN(c2ccccc2)C(=O)CN1Cc1cncn1Cc1ccc(Br)cc1]

Compound 2m was prepared via the synthetic procedure noted for compound 2f described above, except that compound 31 was used instead of compound 6. Yield: 56% (65 mg on a 0.24 mmol reaction scale). Colorless oil. Purity: 99% by HPLC. ¹H NMR (CD₃OD, 500 MHz) δ 9.09 (s, 1H), 7.70 (s, 1H), 7.63 (d, J=8.2 Hz, 2H), 7.46 (t, J=7.6 Hz, 2H), 7.35 (d, J=7.4 Hz, 1H), 7.26-7.22 (m, 4H), 5.60 (s, 2H), 3.94 (d, J=14.6 Hz, 1H), 3.85 (d, J=14.6 Hz, 1H), 3.69 (dd, J=4.0, 12.4 Hz, 1H), 3.42 (dd, J=4.6, 12.4 Hz, 1H), 3.41 (d, J=17.4 Hz, 1H), 3.26 (d, J=17.4 Hz, 1H), 3.03-2.99 (m, 1H), 1.73-1.67 (m, 1H), 1.61-1.54 (m, 1H), 1.41-1.28 (m, 4H), 0.94 (t, J=6.9 Hz, 3H). ¹³C{¹H} NMR (CD₃OD 125 MHZ) δ 169.8, 143.9, 139.4, 135.9, 134.3, 133.2, 131.3, 131.0, 129.4, 128.1, 124.6, 122.1, 58.4, 54.5, 54.1, 52.0, 47.6, 30.7, 27.8, 24.6, 15.1. HRMS (ESI-TOF): Calcd for (C₂₅H₃₀BrN₄O)⁺ 481.1598; found: 481.1641.

The intermediate Compound 31 was prepared as follows.

### a. Synthesis of tert-Butyl(S)-(1-(phenylamino) hexan-2-yl) carbamate (29)

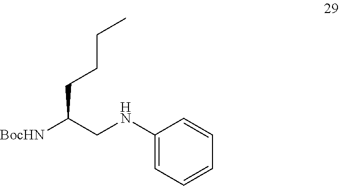 [SMILES: CCCC[C@@H](CNc1ccccc1)NC(=O)OC(C)(C)C]

Compound 29 was prepared using the procedure noted for compound 4 described above, except that aniline was used instead of 3-(trifluoromethoxy) aniline. Yield: 60% (756 mg on a 4 mmol reaction scale). Pale yellow oil. ¹H NMR (CDCl₃, 500 MHZ) δ 7.16 (t, J=8.5 Hz, 2H), 6.67 (t, J=7.3 Hz, 1H), 6.6 (t, J=7.7 Hz, 2H), 4.45 (bs, H), 4.08 (bs, H), 3.81 (bs, 1H), 3.24-3.21 (m, 1H), 3.06-3.02 (m, 1H), 1.50-1.56 (m, 1H), 1.45 (s, 9H), 1.41-1.31 (m, 5H), 0.91 (t, J=6.9 Hz, 3H). HRMS (ESI-TOF): Calcd for (C₁₇H₂₈N₂NaO₂)⁺ 315.2043; found: 315.2051.

### b. Synthesis of tert-Butyl(S)-2-butyl-5-oxo-4-phenylpiperazine-1-carboxylate (31)

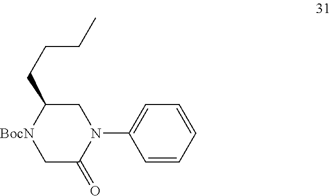 [SMILES: CCCC[C@H]1CN(c2ccccc2)C(=O)CN1C(=O)OC(C)(C)C]

Compound 31 was prepared via the synthetic procedure for compound 6 described above except that compound 29 was used instead of compound 4. Yield: 80% (568 mg on a 2 mmol reaction scale). Pale yellow oil. ¹H NMR (CDCl₃, 500 MHz) δ 7.43-7.40 (m, 2H), 7.31-7.24 (m, 3H), 4.43 (bs, 2H), 4.03 (dd, J=4.1, 12.4 Hz, 1H), 3.91 (d, J=18.7 Hz, 1H), 3.44 (d, J=12.3 Hz, 1H), 1.85-1.77 (m, 1H), 1.68-1.60 (m, 1H), 1.50 (s, 9H), 1.41-1.25 (m, 4H), 0.92 (t, J=7.0 Hz, 3H). HRMS (ESI-TOF): Calcd for (C₁₉H₂₈N₂NaO₃)⁺ 355.1992; found: 355.1991.

### Example 19

Synthetic route for the preparation of 2n.

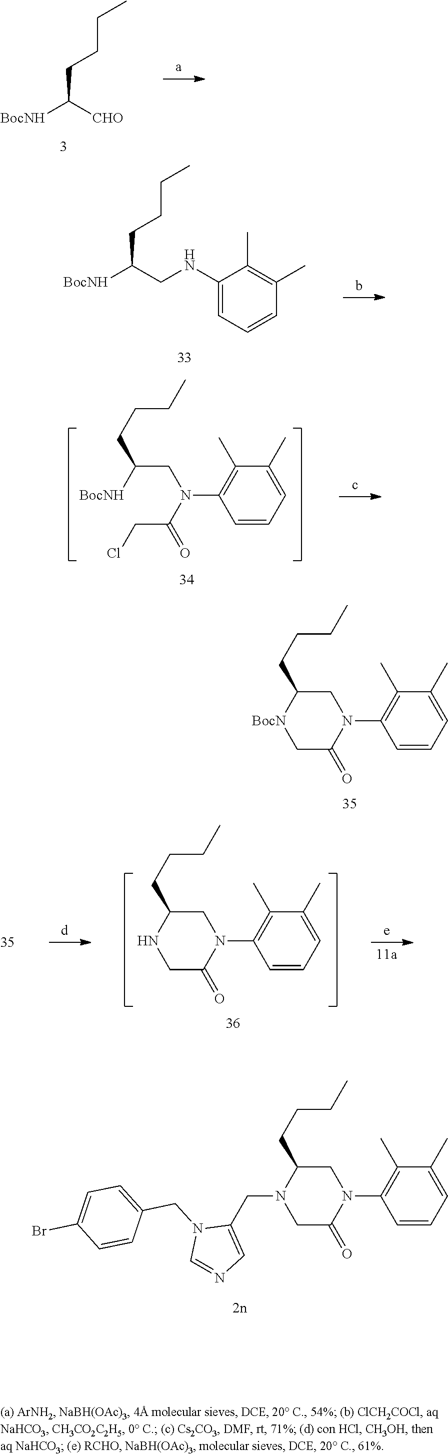

### Example 19. Synthesis of(S)-4-((5-((2-butyl-4-(2,3-dimethylphenyl)-5-oxopiperazin-1-yl)methyl)-1H-imidazol-1-yl)methyl)benzonitrile (2n)

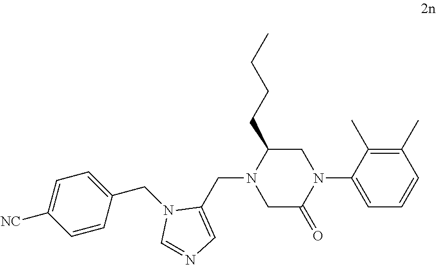 [SMILES: CCCC[C@H]1CN(c2cccc(C)c2C)C(=O)CN1Cc1cncn1Cc1ccc(C#N)cc1]

Compound 2n was prepared via the synthetic procedure noted for compound 2f described above, except that compound 35 was used instead of compound 6. Yield: 61% (56 mg on a 0.2 mmol reaction scale). Colorless oil. Purity: 95% by HPLC. ¹H NMR (CD₃OD, 500 MHz) δ 8.91 (s, 1H), 7.69 (d, J=7.9 Hz, 2H), 7.41 (s, 1H), 7.26 (d, J=7.9 Hz, 2H), 7.17-7.14 (m, 2H), 6.94-6.86 (m, 1H), 5.58 (s, 2H), 3.81-3.55 (m, 3H), 3.46-3.23 (m, 3H), 2.91-2.87 (m, 1H), 2.29 (s, 3H), 2.06 (s, 3H), 1.66-1.53 (m, 1H), 1.37-1.22 (m, 4H), 0.91-0.87 (m, 3H). ¹³C NMR (CD₃OD 125 MHz) δ 166.4 (166.3), 139.8 (139.7), 138.78 (138.75), 138.35 (138.32), 137.7, 133.5 (133.4), 133.2, 130.1, 129.4 (129.3), 127.74 (127.70), 127.0 (126.9), 124.2 (124.1), 121.1 (121.0), 117.8, 113.3, 56.4 (56.1), 52.6, 52.1 (52.0), 50.2, 48.2 (45.9), 28.55 (28.50), 26.4 (25.9), 22.66 (22.64), 20.3, 13.9 (13.7), 13.8. HRMS (ESI-TOF): Calcd for (C₂₈H₃₄N₅O)⁺ 456.2758; found: 456.2750.

The intermediate compound 35 was prepared as follows.

### a. Synthesis of tert-Butyl(S)-(1-((2,3-dimethylphenyl)amino)hexan-2-yl)carbamate (33)

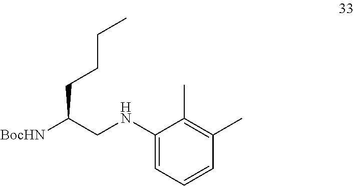 [SMILES: CCCC[C@@H](CNc1cccc(C)c1C)NC(=O)OC(C)(C)C]

Compound 33 was prepared using the the synthetic procedure noted for compound 4 described above, except that 2,3-dimethylaniline was used instead of 3-(trifluoromethoxy)-aniline. Yield: 54% (556 mg on a 3 mmol reaction scale). Pale yellow oil. ¹H NMR (CDCl₃, 500 MHz) δ 7.00 (t, J=7.6 Hz, 1H), 6.56 (d, J=7.5 Hz, 1H), 6.45 (d, J=7.7 Hz, 1H), 4.46 (bs, 1H), 4.20 (bs, 1H), 3.90 (bs, 1H), 3.25-3.55 (m, 1H), 3.02-2.99 (m, 1H), 2.27 (s, 3H), 2.04 (s, 3H), 1.61-1.57 (m, 1H), 1.45 (s, 9H), 1.49-1.31 (m, 5H), 0.91 (t, J=6.8 Hz, 3H). HRMS (ESI-TOF): Calcd for (C₁₉H₃₂N₂NaO₂)⁺ 343.2356; found: 343.2355.

### b. Synthesis of tert-Butyl(S)-2-butyl-4-(2,3-dimethylphenyl)-5-oxopiperazine-1-carboxylate (35)

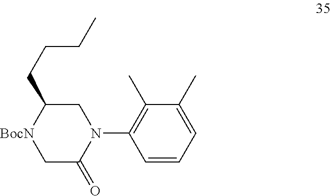 [SMILES: CCCC[C@H]1CN(c2cccc(C)c2C)C(=O)CN1C(=O)OC(C)(C)C]

Compound 35 was prepared using the procedure noted for compound 6 described above, except that compound 33 was used instead of compound 4. Yield: 71% (408 mg on a 1.5 mmol reaction scale). Pale yellow oil. ¹H NMR (CDCl₃, 500 MHz) δ 7.18-7.13 (m, 2H), 6.98-6.90 (m, 1H), 4.58-4.35 (m, 2H), 3.97-3.83 (m, 2H), 3.31-3.20 (m, 1H), 2.31 (s, 3H), 2.13 (s, 1.5H), 2.10 (s, 1.5H), 1.90-1.86 (m, 1H), 1.71-1.67 (m, 1H), 1.50 (s, 9H), 1.46-1.31 (m, 4H), 0.95-0.91 (m, 3H). HRMS (ESI-TOF): Calcd for (C₂₁H₃₂N₂NaO₃)⁺ 383.2305; found: 383.2298.

### Examples 20-23

Synthetic route for the preparation of 2t-w.

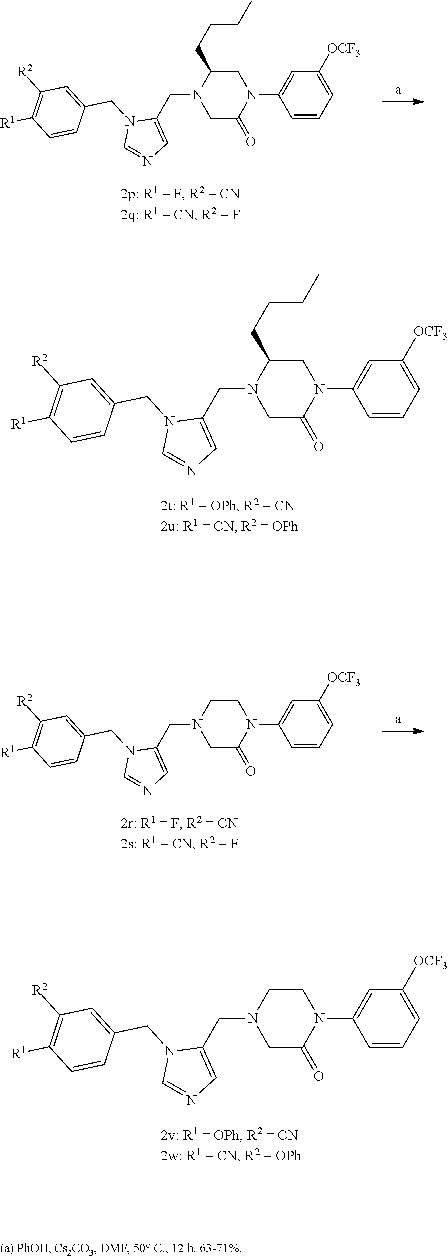

### Example 20. Synthesis of(S)-5-((5-((2-Butyl-5-oxo-4-(3-(trifluoromethoxy)phenyl)-piperazin-1-yl)methyl)-1H-imidazol-1-yl)methyl)-2-phenoxybenzonitrile (2t)

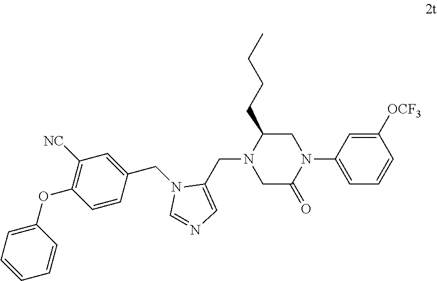 [SMILES: CCCC[C@H]1CN(c2cccc(OC(F)(F)F)c2)C(=O)CN1Cc1cncn1Cc1ccc(Oc2ccccc2)c(C#N)c1]

To a DMF solution (2 mL) of 2p (95 mg, 0.18 mmol) above was added phenol (169 mg, 1.8 mmol), and Cs₂CO₃ (70.4 mg, 0.22 mmol), and the mixture was kept at 50° C. for 24 h. The reaction mixture was quenched with aqueous NH₄Cl solution, and extracted with EtOAc (3×50 mL), the combined extracts were dried over anhydrous MgSO₄, filtered, and concentrated under reduced pressure. The final product was purified using preparative HPLC (Solvent A: H₂O—0.1% TFA, Solvent B: ACN—0.1% TFA, Solvent gradient: 0% B—5 min followed by 0-100% B over 60 min) to afford pure product in 71% yield (77 mg) as colorless oil. Purity: 98% by HPLC. ¹H NMR (CD₃OD, 500 MHz) δ 9.1 (s, 1H), 7.81 (d, J=2.3 Hz, 1H), 7.68 (s, 1H), 7.57-7.51 (m, 2H), 7.46-7.43 (m, 2H), 7.35-7.32 (m, 2H), 7.30-7.25 (m, 2H), 7.09-7.07 (m, 2H), 6.93 (d, J=8.8 Hz, 1H), 5.60 (d, J=2.5 Hz, 2H), 3.97 (d, J=14.6 Hz, 1H), 3.86 (d, J=14.6 Hz, 1H), 3.84 (dd, J=4.1, 12.2 Hz, 1H), 3.54 (dd, J=5.1, 12.2 Hz, 1H), 3.33 (d, J=17.4 Hz, 1H), 3.24 (d, J=17.5 Hz, 1H), 3.08-3.06 (m, 1H), 1.76-1.69 (m, 1H), 1.53-1.56 (m, 1H), 1.42-1.31 (m, 4H), 0.93 (t, J=6.9 Hz, 3H). ¹³C NMR (CD₃OD 125 MHZ) δ 167.6, 160.0, 154.6, 149.2 (q, J=3.0 Hz), 143.1, 137.1, 133.7, 133.1, 130.8, 130.3, 130.1, 129.2, 125.3, 124.1, 120.4 (q, J=253.7 Hz), 120.0, 119.7, 119.3, 118.8, 117.1, 114.8, 103.6, 56.3, 52.1, 51.8, 49.1, 45.2, 28.4, 26.1, 22.4, 12.9. ¹⁹F{¹H} NMR (CD₃OD, 470 MHz) δ −59.4. HRMS (ESI-TOF): Calcd for (C₃₃H₃₃F₃N₅O₃)⁺ 604.2530; found: 604.2520.

### Example 21. Synthesis of(S)-4-((5-((2-butyl-5-oxo-4-(3-(trifluoromethoxy)phenyl)-piperazin-1-yl)methyl)-1H-imidazol-1-yl)methyl)-2-phenoxybenzonitrile (2u)

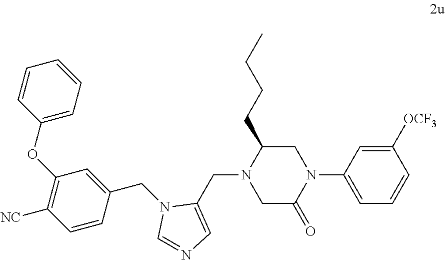 [SMILES: CCCC[C@H]1CN(c2cccc(OC(F)(F)F)c2)C(=O)CN1Cc1cncn1Cc1ccc(C#N)c(Oc2ccccc2)c1]

Compound 2u was prepared via the synthetic procedure noted for compound 2t described above, except that compound 2q was used instead of compound 2p. Yield: 65% (55 mg on a 0.14 mmol reaction scale). Colorless oil. Purity: 99% by HPLC. ¹H NMR (CD₃OD, 500 MHz) δ 9.1 (s, 1H), 7.83 (d, J=8.0 Hz, 1H), 7.68 (s, 1H), 7.60-7.57 (m, 1H), 7.49 (t, J=7.8 Hz, 2H), 7.32-7.29 (m, 4H), 7.14 (d, J=7.9 Hz, 2H), 7.08 (d, J=7.9 Hz, 1H), 6.77 (s, 1H), 5.61 (s, 2H), 3.87 (d, J=14.6 Hz, 1H), 3.79 (d, J=14.6 Hz, 1H), 3.69 (dd, J=4.1, 12.2 Hz, 1H), 3.41 (dd, J=4.8, 12.2 Hz, 1H), 3.26 (d, J=17.5 Hz, 1H), 3.13 (d, J=17.5 Hz, 1H), 2.99-2.92 (m, 1H), 1.69-1.62 (m, 1H), 1.56-1.49 (m, 1H), 1.42-1.25 (m, 4H), 0.93 (t, J=7.1 Hz, 3H). ¹³C NMR (CD₃OD 125 MHZ) δ 169.7, 162.5, 156.9, 151.6 (q, J=3.0 Hz), 145.3, 144.3, 139.7, 136.9, 133.1, 132.7, 132.5, 127.7, 126.5, 123.3, 122.7 (q, J=253.2 Hz), 122.3, 122.1, 121.6, 121.1, 117.2, 116.9, 105.5, 58.5, 54.0, 53.9, 51.9, 47.5, 30.6, 28.1, 24.6, 15.1. ¹⁹F{¹H} NMR (CD₃OD, 470 MHz) δ −59.5. HRMS (ESI-TOF): Calcd for (C₃₃H₃₃F₃N₅O₃)⁺ 604.2530; found: 604.2516.

### Example 22. Synthesis of 4-((5-((3-Oxo-4-(3-(trifluoromethoxy)phenyl) piperazin-1-yl)methyl)-1H-imidazol-1-yl)methyl)-2-phenoxybenzonitrile (2v)

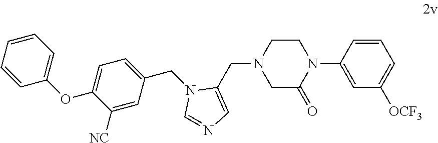 [SMILES: N#Cc1cc(Cn2cncc2CN2CCN(c3cccc(OC(F)(F)F)c3)C(=O)C2)ccc1Oc1ccccc1]

Compound 2v was prepared via the synthetic procedure noted for compound 2t described above, except that compound 2r was used instead of compound 2p. Yield: 66% (72 mg on a 0.2 mmol reaction scale). Colorless oil. Purity: 98% by HPLC. ¹H NMR (CD₃OD, 500 MHz) δ 9.1 (s, 1H), 7.85 (s, 1H), 7.69 (s, 1H), 7.55 (t, J=8.5 Hz, 2H), 7.44 (t, J=7.7 Hz, 2H), 7.36-7.52 (m, 2H), 7.30-7.26 (m, 2H), 7.07 (d, J=7.9 Hz, 2H), 6.94 (d, J=8.7 Hz, 1H), 5.61 (s, 2H), 3.77 (s, 2H), 3.66 (t, J=5.1 Hz, 2H), 3.22 (s, 2H), 2.91 (t, J=5.4 Hz, 2H). ¹³C NMR (CD₃OD 125 MHz) δ 169.3, 162.2, 156.9, 151.5 (q, J=3.0 Hz), 145.2, 139.5, 136.1, 135.6, 132.5, 132.4, 132.3, 131.8, 127.5, 126.4, 122.7 (q, J=256.2 Hz), 122.6, 122.5, 121.1, 119.2, 117.2, 105.8, 58.4, 51.7, 51.3, 51.0, 50.8. ¹⁹F{¹H} NMR (CD₃OD, 470 MHz) δ −59.5. HRMS (ESI-TOF): Calcd for (C₂₉H₂₅F₃N₅O₃)⁺ 548.1904; found: 548.1905.

### Example 23. Synthesis of 4-((5-((3-Oxo-4-(3-(trifluoromethoxy)phenyl) piperazin-1-yl)methyl)-1H-imidazol-1-yl)methyl)-2-phenoxybenzonitrile (2w)

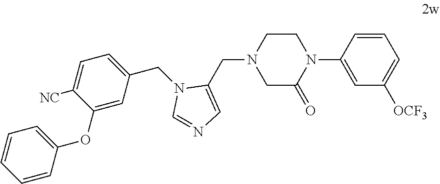

Compound 2w was prepared via the synthetic procedure noted for compound 2t described above, except that compound 2s was used instead of compound 2p. Yield: 63% (52 mg on a 0.15 mmol reaction scale). Colorless oil. Purity: 97% by HPLC. ¹H NMR (CD₃OD, 500 MHz) δ 9.1 (s, 1H), 7.82 (d, J=8.0 Hz, 1H), 7.68 (s, 1H), 7.57 (t, J=8.0 Hz, 1H), 7.47 (t, J=7.7 Hz, 2H), 7.31-7.28 (m, 4H), 7.14 (d, J=7.8 Hz, 2H), 7.01 (d, J=8.0 Hz, 1H), 6.82 (s, 1H), 5.61 (s, 2H), 3.65 (s, 2H), 3.47 (t, J=5.2 Hz, 2H), 3.18 (s, 2H), 2.78 (t, J=5.5 Hz, 2H). ¹³C NMR (CD₃OD 125 MHz) δ 169.1, 162.5, 157.0, 151.5 (q, J=3.0 Hz), 145.1, 144.4, 139.8, 136.8, 132.6, 132.5, 127.6, 126.5, 123.4, 122.7 (q, J=253.2 Hz), 122.3, 122.1, 121.6, 121.1, 117.5, 117.0, 105.4, 58.3, 52.1, 51.6, 50.9, 50.6. ¹⁹F{¹H} NMR (CD₃OD, 470 MHz) δ −59.4. HRMS (ESI-TOF): Calcd for (C₂₉H₂₅F₃N₅O₃)⁺ 548.1904; found: 548.1892.

### Example 24. Biological Assays

### Results

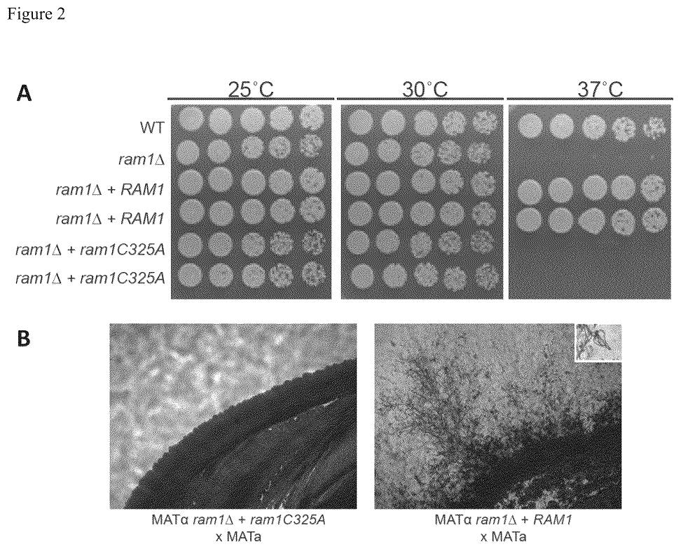

An active site point mutant confirmed that CnFTase is a good drug target. A C. neoformans strain in which the CnFTase β subunit was deleted (ram1Δ) neither survived above 37° C., nor was virulent in animal models, nor exhibited the filamentous differentiation required for fungal mating (Esher, S. K, et al., mSphere, 2016, 1; and Vallim, M. A, et al., Microbiology, 2004, 150, 1925-35). These phenotypes are consistent with lack of Ras farnesylation (Nichols, C. B, et al., J Biol Chem, 1996, 271, 28541-8). To pinpoint these phenotypes specifically to loss of CnFTase activity, rather than another mechanism that involves participation of the β subunit in formation of unknown protein complexes, an intact but enzymatically inactive holoenzyme was constructed by introducing a C325A point mutation into the β subunit (ram1-C325A). This mutation prevents binding of the catalytically essential Zn²⁺ cofactor, resulting in loss of all enzyme activity (Kral, A. M, et al., J Biol Chem, 1997, 272, 27319-23). Transformation of ram1-C325A into the ram1Δ mutant strain restored neither thermotolerant growth nor mating competence, whereas full restoration was observed by complementation with wild-type enzyme in ram1Δ+RAM1 transformants as expected (FIG. 2).

The inability of the inactive mutant holoenzyme to support the Ras activities essential for C. neoformans pathogenesis shows that developing small molecule inhibitors which target the active site is an appropriate strategy for eliminating CnFTase activity.

The complex of CnFTase with L-778,123 suggested four points for lead elaboration. L-778,123 is a linear, flexible four-ring system (FIG. 1B). It binds to the catalytic Zn²⁺ ion at the center of the active site through chelation of the B ring imidazole (FIG. 1C) (Hast, M. A, et al., J Biol Chem, 2011, 286, 35149-62). Two arms of unequal size flank the chelated ring, the smaller of which comprises the A ring, the larger the C and D rings. The small arm interacts predominantly with the FPP substrate; the large arm with the product exit groove (C ring) and both the peptide- and FPP-binding sites (D ring). The two arms fold together in the enzyme active site such that their tips meet at the junction between the FPP- and peptide-binding sites, with the A, B and C rings forming a compact core. The D ring is located on the outside of this core and is free to rotate.

In CnFTase, L-778,123 forms extensive interactions with the entire aliphatic chain of the bound FPP (Hast, M. A, et al., J Biol Chem 2011, 286, 35149-62). The face of the A ring on the surface of the core stacks against isoprene 2 (FIG. 1C). Furthermore, the flexible D ring adopts a g⁻ rotamer such that the fluorine at its tip forms a non-covalent halogen bond (Cavallo, G, et al., Chemical Reviews, 2016, 116, 2478-2601) with the terminus of isoprene 3.

Analysis of this complex indicated four synthetically accessible points for further elaboration of L-778,123, which each explore different aspects of the enzyme-inhibitor interface: the R¹ position points towards the peptide substrate binding site; R² points towards the product exit groove which exhibits the greatest structural and sequence diversion between species (Mabanglo, M. F, et al., Protein Sci, 2014, 23, 289-301; Hast, M. A.; Beese, L. S., J Biol Chem, 2008, 283, 31933-40; and Hast, M. A, et al., J Biol Chem, 2011, 286, 35149-62); the R³ position abuts the FPP substrate binding site; the R⁴ position points at the junction between the peptide and the FPP substrates, and therefore can potentially modulate binding of either ligand (or both). The R¹ and R⁴ positions are adjacent to each other in the CnFTase active site, located at the tips of the long and short arms, respectively.

Synthesis of L-778,123 derivatives. A focused collection of L-778, 123-related compounds was prepared by modifying all four positions singly and in several combinations (FIG. 10), initially using a route developed at Merck (Williams, T. M, et al., J Med Chem, 1999, 42, 3779-3784) and later via a more efficient approach (Suryadevara, P. K, et al., J Med Chem, 2009, 52, 3703-3715).

The library of 23 compounds contained leads with 60- to 150-fold enhanced ^{1μM}IC₅₀ values. The affinities of the new compounds were initially estimated by measuring the concentration at which inhibition resulted in half-maximal enzyme rates at 1 μM FPP (^{1μM}IC₅₀), using an assay based on farnesylation of a fluorescent peptide (Cassidy, P. B, et al., Methods Enzymol, 1995, 250, 30-43). More than two-thirds of the compounds exhibited at least six-fold enhanced ^{1μM}IC₅₀ values compared to the starting point, with the top two exhibiting 60- (2a) and 150-fold (2q) improvements (Table 1, FIG. 10).

Five leads exhibited MIC values superior to an antifungal in clinical use. Antifungal activity was determined by measuring minimum inhibitory concentrations (MIC) of C. neoformans by two methods: a solid medium disc diffusion assay and broth microdilution assay. Ten compounds exhibited measurable antifungal activity to various degrees, whereas L-778,123 did not (Table 1, FIG. 10). Five compounds (Compound 2f, Compound 21, Compound 2e, Compound 2h, and Compound 2k) had MIC values (3-6 μM) that are superior to fluconazole (8 μg/mL or more, i.e. ≥26 μM; Bongomin, F, et al., Mycoses 2018, 61, 290-297).

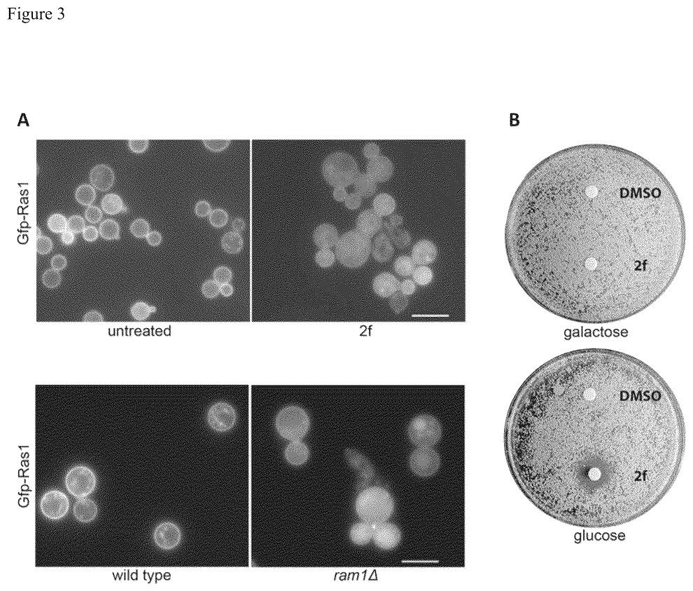

Compound 2f inhibited FTase activity in vivo. At sub-lethal concentrations of the most effective anti-fungal compound in our collection, Compound 2f, an N-terminal fusion between Ras1 and green fluorescent protein (Gfp-Ras1) does not localize to the plasma membrane (FIG. 3A), consistent with loss of farnesylation (Nichols, C. B, et al., J Biol Chem, 1996, 271, 28541-8). Over-expression of CnFTase using a galactose-inducible RAM1 construct negated antifungal activity of Compound 2f (FIG. 3B). Both effects are consistent with CnFTase being the target of Compound 2f, as expected.

Compound 2f is subject to efflux effects in Schizosaccharomyces pombe. The S. pombe fission yeast has been used extensively to test the effect of efflux systems on antifungal activities (Kawashima, S. A, et al., Chem Biol, 2012, 19, 893-901). Wild-type S. pombe was sensitive to Compound 2f, with a MIC of 12.5 μM. In the efflux-defective strain MJ-1682 the MIC improved eight-fold to 1.5 μM. The efficacy of Compound 2f therefore is limited by efflux systems, like other antifungals.

Compound 2f is relatively less toxic to cultured mammalian cells. The MIC of Compound 2f for J774A. 1 macrophage-like murine cells was found to be 200 μM (close to its maximal solubility in aqueous solution). This ˜30-fold decrease in toxicity relative to the pathogen suggests that it may be possible to establish a therapeutic window in animals.

MIC and ^{1μM}IC₅₀ values are not correlated. Surprisingly, there was no direct correlation between the in vitro ^{1μM}IC₅₀ and in vivo MIC values of the compounds (Table 1, FIG. 10). For instance, Compound 2q had the best ^{1μM}IC₅₀ value (0.04 μM; 150-fold improvement over L-778,123), but barely exhibited antifungal activity, whereas the compounds with the best antifungal activity (Compound 2f and Compound 21) showed lesser ^{1μM}IC₅₀ improvements (30-40 fold). The analysis of enzyme kinetics and the structures of CnFTase-inhibitor complexes revealed that this phenomenon is a consequence of differences in their inhibitory mechanisms (see below).

Phosphate is an allosteric effector that alters FPP affinity, catalytic rate, and ^{1μM}IC₅₀. Since phosphate can modulate the kinetics of prenyl transferases (Scholten, J. D, et al., Journal of Biological Chemistry, 1997, 272, 18077-18081; and Huber, H. E, et al., Journal of Biological Chemistry, 2001, 276, 24457-24465), its effects on the steady-state kinetics of CnFTase (FIG. 4) were investigated with respect to FPP and the _{1μM}IC₅₀ values of two interesting compounds, Compound 2f (best MIC) and Compound 2q (best ^{1μM}IC₅₀). At concentrations above 0.1 mM phosphate switches CnFTase to a state exhibiting an eight-fold improved FPP-binding affinity (FIG. 4B) and higher maximal velocity (FIG. 4C) with improved ^{1μM}IC₅₀ values for both inhibitors (FIGS. 4E and H). This activated state is dominant at 10 mM phosphate; at higher concentrations phosphate is weakly inhibitory.

A statistical thermodynamic model that is consistent with these observations (FIGS. 4J and K) was developed by applying the logic of conformational coupling in complex systems (Allert, M. J.; Hellinga, H. W., J Mol Biol, 2020, 432, 1926-1951) to account for the phosphate dependencies of the enzyme properties. In this model, phosphate acts as an allosteric effector that binds at an unknown location (aP) outside the active site and mediates a two-state transition between two CnFTase conformations, L(ow phosphate), and H(igh phosphate), which differ in substrate binding (K_{M}), catalytic efficiency (Vₘₐₓ), and the ^{1μM}IC₅₀ values of Compound 2f and Compound 2q. At another location, within the active site, ψP, phosphate also acts as a weak product-analog inhibitor (the true product is pyrophosphate).

In the absence of phosphate, L dominates; phosphate binding at aP shifts the equilibrium to H (FIG. 4J). The apparent Vₘₐₓ, K_{M}, and ^{1μM}IC₅₀ values therefore shift upon addition of phosphate as the weighted average of the fraction of the two conformations f_{L} and f_{H},

$$e . g . {}_{}^{app} {K}_{M}^{} = {f}_{L} {}_{}^{L} {K}_{M}^{} + {f}_{H} {}_{}^{H} {K}_{M}^{} .$$

Pseudo-product inhibition occurs only in the H state with occupied aP (FIG. 4K), because the affinity of aP is ˜500-fold higher than ψP. Consequently, in the absence of inhibitors, Vₘₐₓ values peak at intermediate phosphate concentrations where aP is fully occupied and H is dominant, but ψP is still empty, and decrease at high phosphate where both aP and ψP are occupied (FIGS. 4C, F and I).

Competitive and uncompetitive binding modes of 2f and 2q. The effects of Compound 2f and Compound 2q on CnFTase kinetics in the absence (L state) and presence of 10 mM phosphate (H state) were measured. All the Compound 2q data and the Compound 2f data in the presence of phosphate required explicit treatment of the enzyme concentration (“non-ideal” conditions, 9), precluding the use of traditional double reciprocal plots for analyzing inhibition, which require ideal numerical conditions. The data was fit to binding polynomials for competitive (6), uncompetitive (7), and mixed (8) inhibition modes, using a specialized solver (10-13). This analysis (FIG. 5) revealed that Compound 2f and Compound 2q exhibit different inhibition modes (FIG. 4K). Compound 2q is a purely uncompetitive inhibitor: it binds only in the presence of FPP. By contrast Compound 2f exhibits a mixture of two binding modes: uncompetitive like Compound 2q, and a competitive mode that displaces FPP. These inhibitor binding properties are different in the L and H states. For Compound 2q the uncompetitive binding affinity improves ˜10% in the H state (^{H}K_{U}(2q)=4 nM). For 2f the change is more dramatic. In the L state, the uncompetitive and competitive affinities are similar (^{L}K_{U}(2f)=750 nM, ^{L}K_{C}(2f)=320 nM), but in the H state, the competitive affinity improves by ˜100-fold, and is ˜170-fold better than the uncompetitive value (^{H}K_{U}(2f)=500 nM, ^{H}K_{C}(2f)=3 nM). In the presence of phosphate, the binding affinities of Compound 2f and Compound 2q therefore are approximately equal (^{H}K_{C}(2f)=3 nM, ^{H}K_{U}(2q)=4 nM), yet, as discussed below, only Compound 2f is effective in vivo.

The binding model invokes two distinct binding sites for uncompetitive (site I) and competitive (site II) inhibition, as supported by the structural observations (see below). Product inhibition by pyrophosphate occurs only if the FPP substrate is bound and has reacted; binding of phosphate at ψP therefore competitively interferes with FPP binding (FIG. 4K). Consequently, elevated phosphate also alters Compound 2q ^{1μM}IC₅₀ values (FIG. 4H), because uncompetitive inhibition occurs only in the presence of FPP. By contrast, the ^{1μM}IC₅₀ values of competitive inhibitors such as Compound 2f are unaffected (FIG. 4E).

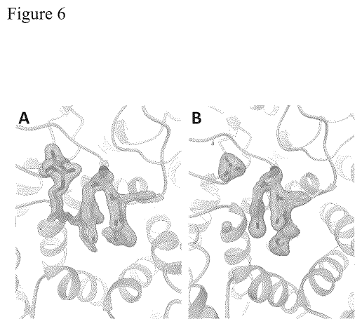

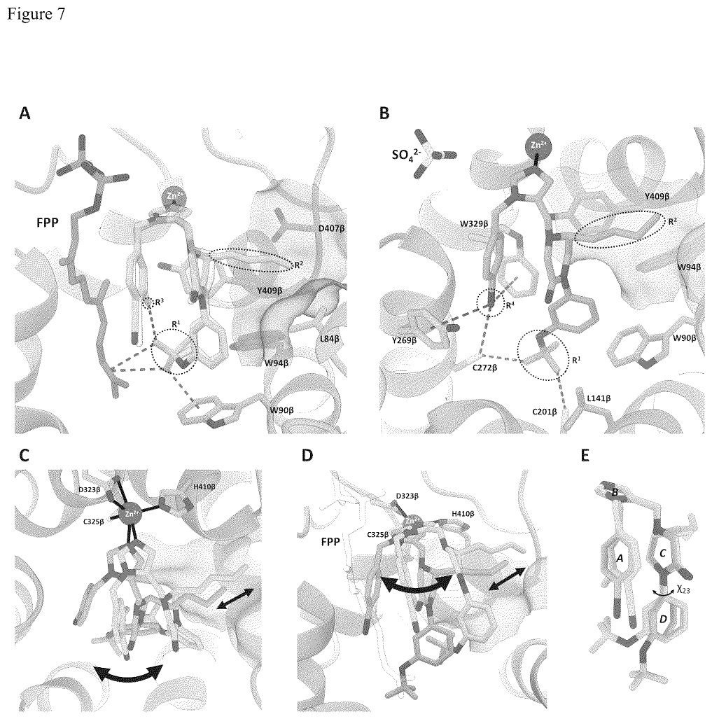

Inhibitors bind at two distinct active-site locations. Structures of seven new compounds bound to CnFTase were determined by X-ray crystallography at 1.8-2.2 Å resolution (FIG. 11). The bound compounds and the presence or absence of FPP substrate were identified unambiguously in electron density maps (FIG. 6). In six structures the new compounds, including Compound 2q, bound in the same location as L-778, 123 (site I). At this site, both inhibitor and FPP are present (FIG. 7A). The compounds retained extensive contacts with FPP via both their A and D rings. Binding at site I is consistent with the uncompetitive binding mode observed in the kinetic analysis of Compound 2q (see above).

In three structures of complexes, including Compound 2f, the compounds bound at a shifted location, site II (FIG. 7B). At this site, the compounds had pivoted by ˜40° around the active site Zn²⁺ (to which the B rings remained chelated) relative to I (FIG. 7C). The D ring adopts two different rotameric conformations in sites I and II, respectively (FIG. 7E; Table 1). These motions place the A ring within the FPP-binding site, thereby sterically preventing FPP from binding. Consequently, bound FPP was not observed if the inhibitor was bound at II. In two complexes (Compound 2e and Compound 2k) a mixture of sites I and II occupancies were observed, and a concomitant partial FPP occupancy. Binding at site II is consistent with the competitive binding mode observed for Compound 2f; the multiple occupancy is consistent with mixed inhibition modes.

### Discussion

Antifungal activity requires competitive inhibition. Steady-state kinetic analysis revealed that Compound 2f and Compound 2q adopt different inhibition modes, comprising uncompetitive (Compound 2q), a mixture of competitive and uncompetitive (Compound 2f in the absence of phosphate), or predominantly competitive (Compound 2f in the presence of 10 mM phosphate) inhibition. Structures of their complexes revealed that the uncompetitive and competitive inhibition modes are explained by binding at sites I or II, respectively. Crystal structures accordingly reveal the inhibition mode of bound compounds.

For the seven complexes that were determined, binding at site II correlates with good MIC values, whereas at site I it never does (Table 1). Antifungal activity therefore requires competitive inhibition, and cannot be achieved with uncompetitive inhibition, even if the inhibitor affinities are approximately the same, as is the case for Compound 2f and Compound 2q in the presence of phosphate (FIG. 5). As explained below, this counterintuitive result is the consequence of the effects of enzyme and substrate concentrations on the efficacy of these two inhibition mechanisms.

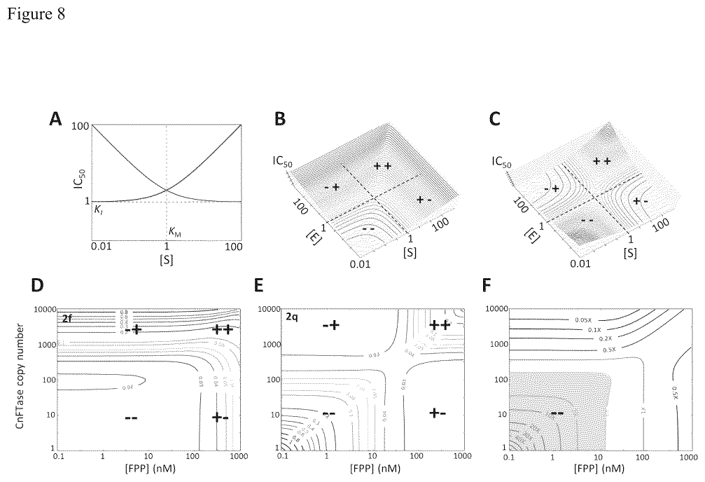

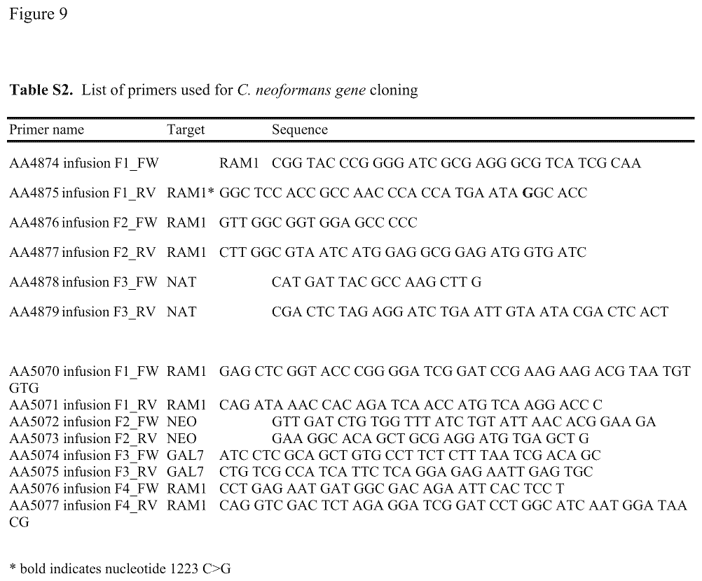

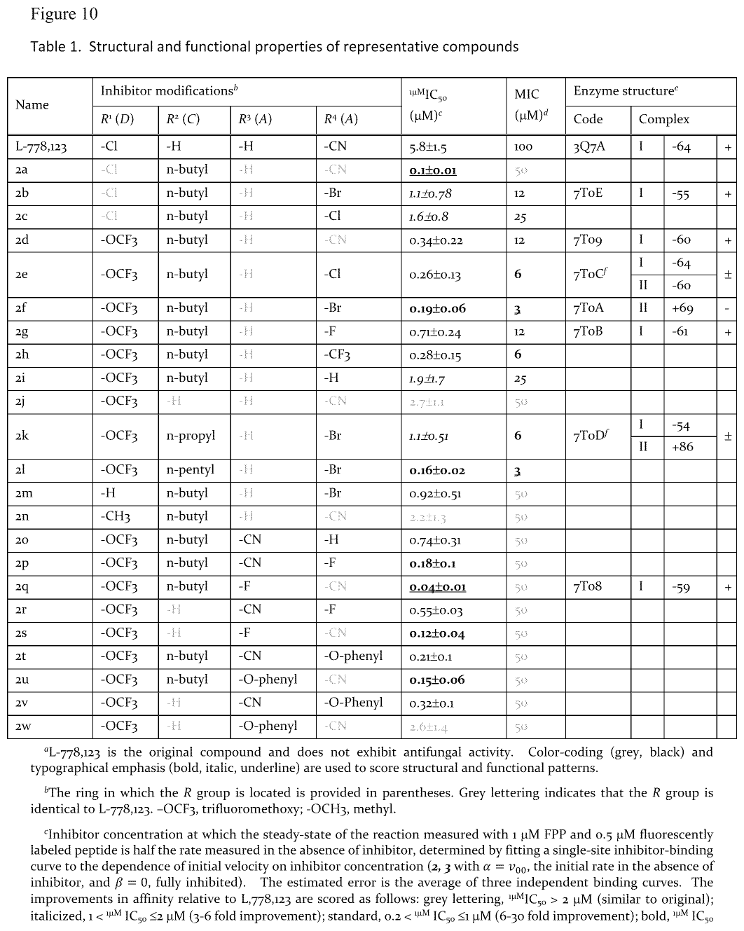

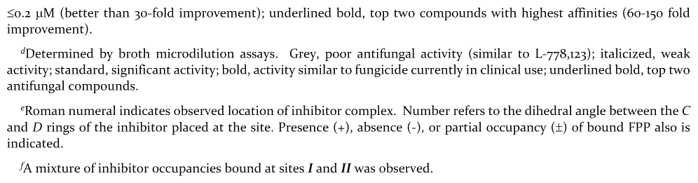

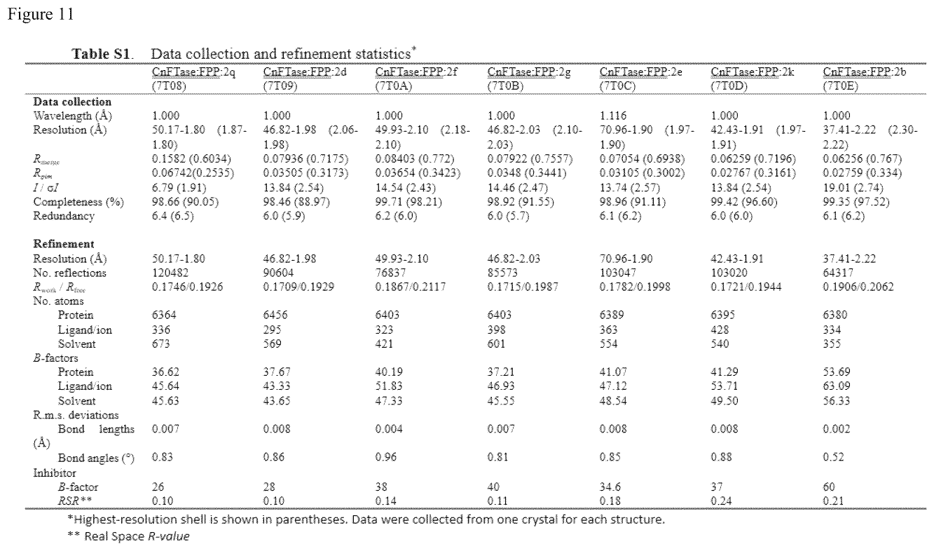

Inhibition mode determines the effect of substrate or enzyme concentration on IC₅₀ values. The key difference between uncompetitive and competitive inhibition is that the former requires substrate binding, and the latter does not. Simulations demonstrated that the two inhibition modes differ in their sensitivity to substrate and enzyme concentrations (FIG. 8).

Enzyme concentrations do not affect steady-state kinetics if the dissociation constants of substrate(s) and inhibitor(s) are much greater than enzyme concentration (9). Under such numerically “ideal” conditions, the efficacy of the two inhibition modes depend only on substrate concentration. At substrate concentrations above the K_{M} value of the enzyme, competitive inhibition becomes increasingly ineffective because the substrate outcompetes the inhibitor; conversely, uncompetitive inhibition loses effectiveness below the K_{M} value, because of loss of the required substrate complex. Competitive and uncompetitive inhibitors therefore are superior in complementary substrate concentration regimes (FIG. 8A).

If numerically “nonideal” conditions apply (9), inhibitor efficacies become dependent on both enzyme and substrate concentrations. Computational tools were developed for simulating non-ideal conditions of arbitrarily complex binding polynomials to model the two inhibition modes (11-13). The simulated multidimensional “phase space” of enzyme and substrate concentrations was divided into “efficacy quadrants” representing IC₅₀ values in the four combinations of pairwise concentration ranges falling above and above the K_{M} value of the enzyme. Competitive inhibition was most effective only at low enzyme and low substrate concentration (−/− quadrant; FIG. 8B). In the other three quadrants, the active enzyme-substrate complex is more stable than the inhibited complex at high concentrations of substrate (+/−), enzyme (−/+), or both (+/+). By contrast, uncompetitive inhibition is most effective in two quadrants (FIG. 8C): low substrate/high enzyme (−/+), and high substrate/low enzyme (+/−). In these two quadrants, the enzyme-substrate complex to which the inhibitor binds prevails, whereas at low substrate/low enzyme (−/−) the concentration of this complex falls below the inhibitor affinity, and at high substrate/high enzyme (+/+), there is a stoichiometric excess of active enzyme-substrate complex relative to inhibitor. Accordingly, if a competitive inhibitor exhibits greater efficacy than an uncompetitive inhibitor, then the combination of in vivo enzyme and substrate concentrations must fall into the −/− quadrant.

Compound 2f is a better inhibitor than Compound 2q at low FPP and CnFTase concentrations likely to prevail in vivo. The H conformation dominates in the presence of 10 mM phosphate, which corresponds approximately to known in vivo phosphate levels (van Eunen, K, et al., FEBS J, 2010, 277, 749-60). In this state, Compound 2f is predominantly competitive, whereas Compound 2q is uncompetitive. Using the substrate and inhibitor affinities of the H state as representative of CnFTase properties in vivo (FIG. 5), the IC₅₀ values of Compound 2f and Compound 2q were modeled over a plausible range of FPP and enzyme intracellular concentrations (FIG. 8D-E). The in vivo enzyme concentration is determined by its cellular copy number. A typical C. neoformans cell is 10 μm in diameter. In such a cell, the concentration of a single protein copy is ˜0.1 nM; 10,000 copies per cell is ˜1 μM. The intracellular FPP concentration is not known for C. neoformans; in cultured 3T3 mouse embryonic fibroblasts it has been estimated at ˜60 nM (Tong, H, et al., Green Chem 2011, 13, 928-933). Therefore FPP concentrations were modeled in the 0.1 nM to 1 μM range. Within these concentration ranges, Compound 2f competitive inhibition accesses the −/− and +/− efficacy quadrants (FIG. 8D), and Compound 2q uncompetitive inhibition the −/+ and +/− quadrants (FIG. 8E). Consequently, Compound 2f outperforms Compound 2q at low FPP levels (<20 nM) and low CnFTase copy numbers (<500) (FIG. 8F). If these limits correspond to upper values for in vivo FPP concentration and CnFTase copy number, then inhibition mode differences explain why Compound 2f is an effective antifungal and Compound 2q is not.

Non-Covalent Halogen Bonds Introduced at R¹ and R⁴ Stabilize Competitive Binding Required for Antifungal Activity

R¹-trifluoromethoxy modifications are necessary for competitive binding, but are ineffective in isolation. Replacement of the R¹—Cl with methyl or trifluoromethoxy (—OCF₃) breaks the halogen bond with the isoprene at the tip of FPP (FIG. 1C), enabling the mobile D ring potentially to form different contacts. By themselves the methyl (Compound 2n) and R¹—OCF3 (2j) modifications had little effect or were slightly deleterious (cf. Compound 2a and Compound 2d). However, in combination with modifications at R⁴, R¹—OCF3 enabled binding at site II.

R²-butyl modifications increase affinity at both sites by improving interactions in the exit-groove. Modifications at R² in the C ring improved affinity for both site I and II binding modes by positioning groups within the exit groove, increasing the interaction surface between inhibitor and enzyme (FIG. 7A). Installation of a butyl group improved affinity about 60-fold, resulting in the second highest affinity compound identified in the entire library (Compound 2a). The length of this modification matters: loss of one methylene unit (propyl) reduced affinity (cf. Compound 2f and Compound 2k); lengthening the group by one methylene unit (pentyl) showed little improvement (cf. Compound 2f and Compound 2l).

R³-halide modifications improve uncompetitive binding through interactions with bound FPP. The R³-proton is part of the interaction surface between the L-778,123 A ring and FPP (FIG. 1C). A halide (2q, 2s) or a bulky-O-phenyl group was introduced (Compound 2u, Compound 2w), within contexts that did (Compound 2q, Compound 2u) or did not (Compound 2s, Compound 2w) contain the R²-butyl group; all contained the R¹—OCF3 group. The R³—O-phenyl modifications did not alter affinities (cf. Compound 2u and Compound 2d or Compound 2w and Compound 2j). The A ring is free to rotate around χ₁₂ (FIG. 1B), enabling R³ modifications to interact with FPP, or point into solvent. It is therefore likely that R³—O-phenyl points into solvent. By contrast, the R³—F halide modification increased the affinities 9-fold (cf. Compound 2q and Compound 2d) or 20-fold (cf. Compound 2s and Compound 2j). This stabilized binding at the uncompetitive site I, and therefore did not result in antifungal activity.

R⁴-halide modifications in combination with R¹—OCF3 stabilize binding at site II. In the presence of R¹—OCF3, replacing the R⁴-nitrile group with halides improved affinity ˜5-fold and enabled binding at site II, with concomitant appearance of potent antifungal activity (cf. Compound 2b with Compound 2f and Compound 2c with Compound 2e). The R¹—OCF3 group on the tip of the rotated D ring replaces isoprene 3 of the now-absent FPP, forming halogen bonds with the surrounding amino acid side-chains (FIG. 7B). At site II, the R⁴ group of the A ring points into a small pocket. Replacement of the R⁴-nitrile group with a halide introduces halogen bonds with three residues in this pocket, intercalating the halide between two aromatic side-chains (FIG. 7B). The size and polarizability of the halide affects the apparent affinity: Br (Compound 2f)>Cl (Compound 2e)>F (Compound 2g). Neither R¹—OCF3 nor R⁴-halide is sufficient to stabilize binding at site II in isolation (cf. Compound 2f, Compound 2b, Compound 2d), but together these modifications successfully encode antifungal activity.

### Conclusions

The structure of the ternary FPP/L-778, 123 complex of CnFTase enabled the identification of four sites in this inhibitor for construction of a focused collection of analogues, within which five derivatives were discovered that, unlike L-778,123, exhibited potent fungicidal activity against C. neoformans with 3-6 μM MIC values that are superior to fluconazole (26 μM), an antifungal that is commonly used to treat this pathogen. Furthermore, these compounds developed resistance mutations less readily than fluconazole. However, their action spectrum is narrower than fluconazole, killing C. neoformans, but not C. albicans or A. fumigatus. The 2f compound was characterized in more detail. Unlike fluconazole, it inhibits C. neoformans biofilms. It still inhibits mammalian FTase, but at about 30-fold worse IC₅₀ values, and is about 30-fold less toxic to cultured mammalian cells relative to fungicidal concentrations.

Characterization of their structures and inhibition mechanisms revealed that the potency of these new antifungals is determined by biophysical effects that render inhibitor efficacies particularly sensitive to both substrate and enzyme concentrations in vivo. The L-778,123 scaffold comprises four flexibly linked rings, three of which (A-C) fold into a compact core, on the surface of which the fourth ring (D) rotates freely. This arrangement enables the inhibitor derivatives to bind at two locations within the enzyme active site, related by a rotation around the catalytic Zn²⁺ to which the B rings are always coordinated. At site I inhibitors form obligate complexes with FPP and therefore exhibit uncompetitive inhibition, whereas binding at site II displaces the FPP substrate resulting in competitive inhibition. L-778,123 and several derivatives bound only at I, whereas others bound at II, or both sites (with a concomitant mixed inhibition mode). Remarkably, only competitively inhibiting derivatives exhibited antifungal activity, whereas uncompetitive derivatives with similar affinities never did.

This phenomenon can be rationalized by the different effects that substrate and enzyme concentration exert on the efficacies of these two inhibition modes. Using solvers for binding polynomials that explicitly include enzyme concentrations in the description of inhibition modes and applying the substrate and inhibitor affinities determined by in vitro steady-state kinetics in the presence of phosphate, the competitive inhibitor 2f was shown to be more effective than the uncompetitive 2q at the low substrate and protein concentration ranges expected to prevail in vivo.

The importance of accounting for likely in vivo conditions was further underlined by the finding that phosphate binding shifts the enzyme into a conformation that improves catalytic activity, enhances FPP affinity, and greatly improves competitive binding at II, but not uncompetitive binding at I. At in vivo phosphate concentrations this conformation dominates; phosphate binding therefore is unlikely to represent a regulatory mechanism. The phosphate-mediated conformational change was an important finding for characterizing and understanding the enzyme, because in the phosphate-free conformation, 2f does not outperform 2q under any combination of enzyme or FPP substrate concentrations.

The affinities of all derivatives, regardless of binding location, were enhanced by introducing a butyl group at R² in the C ring, which introduced additional van der Waals interactions between the inhibitors and the enzyme in the exit groove. The key modifications that determined antifungal activity stabilize a conformation of the flexible D ring, capable of reaching into the FPP-binding site and displacing its substrate. These interactions involved halide modifications, because halogen bonds are compact functional groups able to introduce relatively strong interactions with few geometrical restrictions in tight spaces such as the FTase active site. The bromine at the R⁴ position of the A ring forms a “sticky knob” with a unique, halogen-bonded intercalation geometry between two aromatic amino acid side-chains. The halogen bonds formed by R¹—OCF3 present a “sticky tip” for stabilizing occupancy of the FPP-binding site by the D ring. Only when both these modifications were present was binding at site II achieved to establish competitive inhibition with concomitant antifungal activity.

These findings establish important design criteria for the development of urgently needed antifungals that target farnesyl transferases in C. neoformans and other pathogenic fungi.

### Materials and Methods

Fungal strains and media. The following C. neoformans strains were used: H99⁴⁶, MATα; KN99a⁴⁷, MATa; CBN96²⁶, MATαHIS3-GFP-RAS1-nat; SKE1⁹, MATαram1::neo; CBN121⁹, MATαpHIS3-mChRAS1-nat; CBN460 (this study), MATαram1::RAM1-nat; CBN462 (this study), MATαram1::ram1C325-nat, CBN482 (this study), MATαpGal7-RAM1-neo. The following S. pombe strains were used³³: MJ1682, h90 ade6 leu1 ura4-D18 caf5::bsdR pap1-del pmd1-del mfs1-del bfr 1-del; SAK1, h+ ade6-M210 leu1 ura4-D18. Strains were grown on yeast extract (1%)-peptone (2%)-dextrose (YPD) medium. To induce C. neoformans mating, strains of opposite mating types were co-cultured on MS medium at 25° C. for seven days (Murashige, T, et al., Physiol Plantarum 1962, 15, 473-497).

Molecular Biology. A strain of C. neoformans containing the catalytically inactive C325A allele of the farnesyl was constructed using In-Fusion cloning (Takara; primers in FIG. 9 to fuse ram1C325A to a C. neoformans nourseothricin resistance cassette; a RAM1 control construct was also generated. The ram1C325A:nat (CBN462: ram1Δ+ram1C325A:nat and RAM1:nat (CBN460: ram1Δ+RAM1:nat) constructs were introduced into a ram1Δ mutant strain (SKE1) using biolistic transformation (Toffaletti, D. L, et al., J Bacteriol, 1993, 175, 1405-1411), confirmed by PCR, and assessed for Ram1-dependent phenotypes including temperature sensitivity and mating. To induce C. neoformans mating, strains of opposite mating types were co-cultured on MS medium at 25° C. for seven days (Murashige, T, et al., Physiol Plantarum, 1962, 15, 473-497). A C. neoformans strain with a galactose-inducible RAM1 allele (CBN482) was similarly created with primers designed to create an overlap construct comprising the C. neoformans RAM1 5′ sequence-neomycin resistance cassette-GAL7 promoter-RAM1 coding sequence (FIG. 9) to precisely replace the RAM1 promoter with the GAL7 galactose inducible when introduced into the H99 genome by biolistic transformation.

Microscopy. To assess Ras1 localization, a C. neoformans strain expressing Gfp-Ras1 under a constitutive promoter (CBN96) was incubated overnight in YPD medium (yeast extract, 1% peptone, 2%-dextrose) at 30° C., diluted to OD₆₀₀ 0.1 in YPD, then incubated for 24 hours in serial dilutions of Compound 2f (0.078 μM-6.25 μM) at 37° C. A drug-free control sample contained 1% DMSO. Ras1 localization in a ram1Δ mutant strain was determined in a similar manner: a ram1Δ mutant and wild-type strain, each expressing mCherry-Ras1 under a constitutive promoter (CBN121 and SKE17) were incubated overnight in YPD medium at 30° C., diluted to OD₆₀₀ 0.1 in YPD, then incubated for 24 hours at 37° C. All cells were visualized and imaged by fluorescent microscopy at 1000× magnification using a Zeiss Imager A1 microscope and Zeiss Axio-Cam MRM digital camera. Images were processed using Zeiss Zen and Image J software (Schneider, C. A, et al., Nat Methods 2012, 9, 671-675).

Disc diffusion assay. Overnight cultures of C. neoformans (about 18 hours) were normalized to OD₆₀₀ 0.6, diluted 1/10, and 200 μL of each sample were spread onto either a YPD or YPGal (yeast extract-peptone medium with 3% galactose) plate using sterile glass beads. After drying, 10 μL of compound or solvent control (DMSO) was added to sterile 6 mm filter discs on each plate. Plates were incubated for 48 hours at 37° C.

Minimum inhibitory concentration (MIC) assays. C. neoformans: Overnight cultures (˜18 h) of C. neoformans strains incubated in YPD medium were diluted to 500 cells/ml in RPMI medium buffered with MOPS. Cells were aliquoted (200 μL/well) in black-wall, clear-bottom 96-wells plates (Greiner). Compound stock solutions in DMSO ranged from 9 μM to 20 mM, and added to 200 μL cell suspension aliquoted in 96-well plates. Final aqueous concentrations ranged from 200 μM to 0.09 μM, limiting DMSO to 1%. After 48-hr incubation at 37° C. or 30° C. for 48 h, 10% AlamarBlue (BioRad) was added. The end-point fluorescence intensity of reduced AlamarBlue was measured using a fluorescence plate reader (FLUORStar Optima plate reader) at the time point the indicator turned from blue to pink in healthy control cells.⁵¹ Assays were performed in duplicate. S. pombe MIC assays were performed in a similar manner as for C. neoformans with the modification of substituting YE medium (yeast extract (0.5%)-dextrose (3%)) for YPD and RPMI due to fungal specific growth restrictions.

Macrophage cytotoxicity assays. To measure macrophage cytotoxicity, the inhibitors were prepared in 2-fold serial dilutions in DMSO in a 96-well plate as described for MIC testing. 2 μL of the serially diluted compound were transferred to a 96-well plate containing 200 μL DMEM (100× dilution). J774A.1, a macrophage-like murine cell line, was incubated in DMEM (Dulbecco's Modified Eagle Medium, Sigma-Aldrich) to 70% confluency, harvested, washed in PBS, then transferred to black, clear-bottom 96-well plates (Greiner) at 10⁵ cells/well. Plates were incubated overnight at 37° C. and 5% CO₂. The macrophage medium covering the J774A.1 monolayer was replaced with 100 μL of DMEM containing the serially diluted inhibitors (50× dilution) and incubated for 24 h at 37° C. and 5% CO₂. Macrophage viability was assessed at 24 h by the addition of 10% AlamarBlue and incubating for 3 h at 37° C. and 5% CO₂ prior to fluorescence measurement (FLUORStar Optima plate reader). Assays were performed in duplicate.

Protein expression and purification. The protein was expressed and purified as described previously (Hast, M. A, et al., J Biol Chem, 2011, 286, 35149-62). The purified protein was concentrated (10 mg/ml using a 50 kDa cutoff centrifugal concentrator), exchanged into storage buffer (20 mM HEPES, pH 7.5, 5 μM ZnCl₂, 5 mM DTT), flash-frozen in liquid nitrogen, and stored at −80° C. in small aliquots.

CnFTase activity assay. The farnesyl transferase activity was measured by measuring the time dependence of the fluorescence emission intensity increase due to farnesylation of a dansylated peptide (Cassidy, P. B, et al., Methods Enzymol, 1995, 250, 30-43). Measurements were taken at room temperature in a 96-well black polystyrene, half area, flat bottom, non-binding surface plate using a SpectraMax M5e plate reader (excitation wavelength, 340 nm; emission wavelength 525 nm; emission high-pass filter, 455 nm). Reactions were initiated by addition of CnFTase to reaction mixes, containing variable components (FPP, inhibitor, phosphate) in one of two conditions (A for initial ^{1μM}IC₅₀ determination, and B for all other experiments): Tris-HCl pH 7.5 (A: 62.5 mM; B: 55.5 mM), ZnCl₂ (A: 12.5 μM; B: 11.1 μM) n-dodecyl-β-D-maltoside (A: 0.05% w/v; B: 0.11% w/v), and dansyl-GCVVM (A: 0.62 μM; B: 0.55 μM), TCEP (A: 1.25 mM; B: 1.11 mM). Final CnFTase concentrations (from 5× or 10× stocks in 20 mM Tris-HCl pH7.5, 20 mM NaCl, 5 μM ZnCl₂, 1 mM TCEP, 1 mg/mL BSA): A, 20 nM; B: 4.4, 6.7, 10 nM triplets. FPP (A: 0.125 μM; B, 0.22, 0.33, 0.44, 067, 0.89, 1.33, 2.66, 5.33, 7.10, 10.7, 14.2, 21.3 μM) and compounds stocks were prepared in DMSO; as needed, DMSO was added to reactions: A, 2.5%; B, 2.2%. For experiments that included phosphate, pH 7.5 stock was added to the desired final concentration. Data was analyzed using Python scripts for the Biomolecular Computing (BMC) package, included in the Dryad electronic repository. Data for determining ^{1μM}IC₅₀ values (condition A) was collected in triplicate. Initial rates, vₒ, were extracted by fitting a straight line to the initial to tₘₐₓ seconds of the reaction progress curves, where tₘₐₓ values were selected by inspection and entered into curator tables for automated analysis. _{1μM}IC₅₀ were determined by fitting a single-site Langmuir ligand-binding isotherm to the dependence of vₒ on inhibitor concentration (2, see below). All other experiments (condition B) all other experiments (condition B) used three different CnFTase concentrations, collecting progress curves as a function of FPP, inhibitor, and phosphate concentrations. The resulting triplet of progress curves was used to extract vₒ values automatically by minimizing

$${χ}_{k}^{2} = {\sum }_{i = 1}^{3} {\sum }_{j = 1}^{j = {f}_{i} T} {( {S}_{0 ik} + {v}_{0} {t}_{j} {D}_{i} - {}_{}^{obs} {S}_{jl}^{} )}^{2} \qquad 1$$

where χₖ² is the squared difference of the calculated and observed emissions in the k^{th} iteration of the minimizer, fᵢ the initial fraction (constrained in the interval [0.5,1]) of the T time points of the progress curve, Dᵢ the dilution factor of the i^{th} progress curve (0.44:0.66:1 for condition B), S₀ᵢₖ the fluorescence emission intensity of the i^{th} progress curve at t=0 at the k^{th} (this value is refined individually using a one-dimensional golden section search⁵² with fᵢ and v₀ values of the k−1 iteration), and ^{obs}Sⱼᵢ, the observed intensity of the i^{th} progress curve at the j^{th} time point. The target function 1 is minimized using conjugate gradients with vₒ and fᵢ as the degrees of freedom. The resulting rates were then fit to binding polynomials describing the inhibition mechanism (5-13, see below).

Statistical thermodynamic analysis and modeling. All calculations were carried out using the BMC package. Scripts, data, and results have been provided in the electronic repository.

Single-site binding isotherms. Signals, S, resulting from a binding reaction were fit to

$$S = α ( 1 - \overline{y} ) + β \overline{y} \qquad 2$$

where α and β are the baselines associated with the apo-protein and its ligand complex, respectively, and y̅ fractional occupancy of the binding site

$$\overline{y} = \frac{{K}_{a} x}{1 + {K}_{a} x} \qquad 3$$

where x is the ligand concentration, and Kₐ the association constant of the ligand for the binding site (note the corresponding reciprocal dissociation constant, K_{d}=Kₐ⁻¹, corresponding to the value of x at which y̅=0.5). If x is an inhibitor concentration, then IC₅₀=Kₐ⁻¹. For Michaelis-Menten analysis 2 is reduced to a standard form (Bahar, I.; Jernigan, R. L.; Dill, K. A. Protein Actions, principles & modeling. Garland Science: 2017) with S=vₒ (initial rates), α=0, β=Vₘₐₓ (maximal velocity), and K_{M}=Kₐ⁻¹ (substrate dissociation constant). In general, baseline signals, σ, (α or β) can be constant, or linear functions of ligand concentration x

$$σ = a + bx \qquad 4$$

Binding polynomials for inhibition models. The initial steady-state rates of a reaction involving a single-substrate x, and inhibitor y are given by

$${v}_{0} = \frac{{K}_{a} x}{Q} {V}_{max} \qquad 5$$

where Q is the binding polynomial for the inhibitory mechanism (Bahar, I.; Jernigan, R. L.; Dill, K. A. Protein Actions, principles & modeling. Garland Science: 2017). Three binding polynomials were used:

$$Competitive$$

$${Q}_{c} = 1 + {K}_{a} x + {K}_{z} y \qquad ( 6 )$$

$$Uncompetitive$$

$${Q}_{U} = 1 + {K}_{a} x + {K}_{U} xy \qquad ( 7 )$$

$$and Mixed$$

$${Q}_{M} = 1 + {K}_{a} x + {K}_{C} y + {K}_{U} xy \qquad ( 8 )$$

where Kₐ, K_{C}, and K_{U} are the affinities of the enzyme for substrate, competitive inhibitor, and uncompetitive inhibitor, respectively.

Solvers for ideal conditions. Ideally, free ligand concentrations are well approximated by their total concentrations added to the reaction (x≈x_{T}, y≈y_{T}). This condition holds if the total enzyme concentration p_{T} conditional is true

$${p}_{T} ≪ {K}_{a}^{- 1} , {K}_{C}^{- 1} , {K}_{U}^{- 1} \qquad 9$$

which is usually assumed. Under such ideal conditions the values of Kₐ, K_{c}, K_{U}, Vₘₐₓ in equations 5-8 can be fit using multivariate non-linear least-squares methods that minimize

$${{χ}_{k}^{2} = {\sum }_{1}^{N} {}_{}^{calc} {v}_{o} ( {x}_{i} , {y}_{i} \vert {K}_{a , k} , {K}_{c , k} , {K}_{U , k} , {V}_{max , k} ) - {}_{}^{obs} {v}_{o , i} )}^{2} \qquad 10$$

where for the i^{th} observed initial rate ^{obs}v_{o,i} of N data points, ^{calc}vₒ is calculated with 5 given parameter values K_{a,k}, K_{c,k}, K_{U,k}, V_{max,k} in the k^{th} iteration. Conjugate gradient and simplex methods singly or in combination were used (Allert, M. J.; Hellinga, H. W., J Mol Biol 2020, 432, 1926-1951).

Solvers for non-ideal conditions. In non-ideal conditions 9 is false, and the total protein concentration needs to be included as a degree of freedom, requiring the roots of the following multivariate polynomial to be found (see below for derivation):

$$( x + y ) Q + ( 2 {p}_{T} - {x}_{T} - {y}_{T} ) Q - {p}_{T} ( {Q}_{∼ x} + {Q}_{∼ y} + 2 ) = 0 \qquad 11$$

where Q is one of 6-8, Q_{˜x} and Q_{˜y} are the terms in Q that do not include x and y, respectively (for instance, if Q=Q_{M}, then Q_{˜x}=K_{c}y and Q_{˜y}=Kₐx). Rather than using a generalized solver for multivariate polynomials, the problem was decomposed into univariate simultaneous equations that can be solved individually using Brent's numerical method (Press, W. H. T.; Teukolsky, S. A.; Vetterling, W. T.; Flannery, B. P. Numerical Recipes. The Art of Scientific Computing, 3rd ed.; Cambridge University Press: 2007)

$$xQ + ( {p}_{T} - {x}_{T} ) Q - {p}_{T} ( {Q}_{∼ x} + 1 ) = 0 , \qquad 12$$

$$solve given {y}^{*} and parameters for Q$$

$$yQ + ( {p}_{T} - {y}_{T} ) Q - {p}_{T} ( {Q}_{∼ y} + 1 ) = 0 ,$$

$$solve given {x}^{*} and parameters for Q$$

iteratively solving for roots xᵢ* and yᵢ* roots in the intervals [0, x_{Y}] and [0, y_{T}] until a tolerance ratio, pₘₐₓ, condition has been satisfied

$$\vert log \frac{{x}_{i}^{*} + {y}_{i}^{*}}{{x}_{i - 1}^{*} + {y}_{i - 1}^{*}} \vert - 1 < log {ρ}_{max} \qquad 13$$

starting with estimates x₀*=x_{T} and y₀*=y_{T}. To solve for the parameters in Q (required for data fitting, but not for modeling), 10 is used with xᵢ=xᵢ* and xᵢ=xᵢ*, nesting solvers such that each iteration in 10 requires a complete solution of 11.

Parameter uncertainty estimates. Error estimation was done by bootstrapping, duplicating ˜37% of the observed data points, and re-solving the equations. Errors were estimated as the standard deviations of the resulting parameter distributions (Allert, M. J.; Hellinga, H. W., J Mol Biol, 2020, 432, 1926-1951).

Derivation of univariate non-ideal ligand-binding polynomial solution. For non-ideal conditions where x≈x_{T}. The total protein is the sum of free protein, p, and all liganded species

$$\begin{matrix}{p}_{T} = p + {pK}_{1} x + … \\ = pQ\end{matrix} \qquad 14$$

$$∴ p = \frac{{p}_{T}}{Q} \qquad 15$$

$$where Q = 1 + {K}_{1} x + … \qquad 16$$

a univariate binding polynomial that may contain multiple terms in x. Similarly, the total ligand concentration is the sum of the free ligand and all liganded species

$$\begin{matrix}{x}_{T} = x + {pK}_{1} x + … & \\ = x + p ( Q - 1 ) & ( substituting 16 ) \\ = x + {p}_{T} \frac{( Q - 1 )}{Q} & ( substituting 15 )\end{matrix} \qquad 17$$

$$\begin{matrix}⇒ xQ + ( {p}_{T} - {x}_{T} ) Q - {p}_{T} = 0 & ( rearranging )\end{matrix} \qquad 18$$

Derivation of multivariate non-ideal ligand-binding polynomial solution. For multiple ligands, e.g. substrate x and inhibitor y, 14 was generalized

$$\begin{matrix}{p}_{T} = p + {pK}_{x} x + {pK}_{y} y + {pK}_{xy} xy + … \\ = pQ\end{matrix} \qquad 19$$

$$∴ p = \frac{{p}_{T}}{Q} same as 15$$

where Q now is a multivariate binding polynomial that main contain multiple single or cross terms in x and y. The total concentration of x now is

$$\begin{matrix}{x}_{T} = x + {pK}_{x} x + {pK}_{xy} xy + … \\ = x + p ( Q - 1 - {Q}_{∼ x} ) \\ = x + {p}_{T} \frac{( Q - 1 - {Q}_{∼ x} )}{Q}\end{matrix} \qquad 20$$

$$⇒ xQ + ( {p}_{T} - {x}_{T} ) - {p}_{T} ( {Q}_{∼ x} + 1 ) = 0$$

where Q_{˜x} are all the terms in the binding polynomial that do not include x (e.g. K_{y}y). Similarly, for y,

$$yQ + ( {p}_{T} - {y}_{T} ) - {p}_{T} ( {Q}_{∼ y} + 1 ) = 0 \qquad 21$$

Combining 20 and 21 (which are equivalent to 12), provides the multivariate solution

$$( x + y ) Q + ( 2 {p}_{T} - {x}_{T} - {y}_{T} ) Q - {p}_{T} ( {Q}_{∼ x} + {Q}_{∼ y} + 2 ) = 0 \qquad 22$$

This approach generalizes for n free x₁, x₂, . . . xₙ and total x_{T1}, x_{T2}, . . . x_{Tn} ligands as

$$Q \sum {x}_{i} + ( {np}_{T} - \sum {x}_{Ti} ) Q - {p}_{T} ( n + \sum {Q}_{∼ i} ) = 0 \qquad 23$$

This equation is solved iteratively by decomposing into a system of n equations for each of the ligands, as illustrated for the two-ligand case (12).

Crystallization. CnFTase (10 mg/ml in storage buffer) was mixed with tris [2-carboxyethyl] phosphine (TCEP) pH 7.5 to a final concentration of 5 mM TCEP. Following incubation with 3-fold molar excess of FPP (Sigma) for 30 min on ice, crystals were grown at 17° C. by hanging-drop vapor diffusion (lul protein drop, 0.5 uL reservoir of 100 mM CAPSO pH 9.5, 50-75 mM Li2SO4, 200 mM NaCl, 16%-21% PEG4K). Small crystals formed after one day were crushed with seed beads (Hampton Research, HR2-320) to prepare a microcrystal seed stock, flash-frozen in liquid nitrogen, and stored at −80° C. To obtain crystals of CnFTase-FPP-compound complexes for data collection, the enzyme was pre-incubated with FPP (see above), 3-fold molar excess of compound was added, and incubated (30 minutes on ice). Crystals were grown by hanging-drop vapor diffusion (see above) adding 0.1 ul diluted crystal seeds to each crystallization drop. Resulting crystals were transferred stepwise into a cryoprotection solution (well solution plus ˜30% ethylene glycol) and flash-frozen in liquid nitrogen.

Data collection and structure determination. X-ray diffraction data were collected at SER-CAT Beamline 22-BM, 22-ID at the Advanced Photon Source, Argone National Laboratory, or Advanced Light Source, Lawrence Berkeley National Laboratory. The crystals belonged to the space group P43212 with the unit cell dimensions 141 Å×141 Å×130 Å and one CnFTase heterodimer in the asymmetric unit. CnFTase complex crystals diffracted to approximately 2.0 Å resolution. Structures were solved as described previously (Hast, M. A, et al., J Biol Chem, 2011, 286, 35149-62).

Example 25. The following illustrate representative pharmaceutical dosage forms, containing a compound of formula I (‘Compound X’), for therapeutic or prophylactic use in humans.

| (i) Tablet 1 | mg/tablet |
|---|---|
| Compound X= | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| Compound X= | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Compound X= | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/ml) | mg/ml |
|---|---|
| Compound X = (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/ml) | mg/ml |
|---|---|
| Compound X = (free acid form) | 10.0 |
| Monobasic sodium phosphate | 0.3 |
| Dibasic sodium phosphate | 1.1 |
| Polyethylene glycol 400 | 200.0 |
| 1.0N Sodium hydroxide solution | q.s. |
| (pH adjustment to 7.0-7.5) | |
| Water for injection | q.s. ad 1 mL |

| (vi) Aerosol | mg/can |
|---|---|
| Compound X= | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method comprising, contacting a fungus with an inhibitor of a fungal farnesyl transferase that is competitive with respect to a farnesyl disphosphate substrate.

2. The method of claim 1, wherein the contacting takes place in the presence of an allosteric activator.

3. The method of claim 2, wherein the allosteric activator is a phosphate.

4. The method of claim 2, wherein the allosteric activator is a nucleotide di- or triphosphate, a pyrophosphate, or a polyphosphate.

5. The method of claim 1, wherein the contacting occurs in vitro.

6. The method of claim 1, wherein the contacting occurs in vivo.

7. The method of claim 1, wherein the contacting inhibits the formation or growth of a biofilm.

8. The method of claim 1, wherein the inhibitor of farnesyl transferase binds at site II.

9. The method of claim 8, wherein the binding of the inhibitor of farnesyl transferase at site II displaces farnesyl diphosphate.

10. The method of claim 1, wherein the inhibitor of farnesyl transferase interacts through halogen-bonds with an FTase active site on farnesyl transferase.

11. The method of claim 1, wherein the inhibitor of farnesyl transferase is compound 2f: 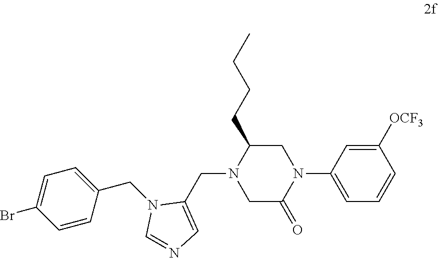 [SMILES: CCCC[C@H]1CN(c2cccc(OC(F)(F)F)c2)C(=O)CN1Cc1cncn1Cc1ccc(Br)cc1]
or a pharmaceutically acceptable salt thereof.

12. The method of claim 1, wherein the inhibitor of farnesyl transferase is: 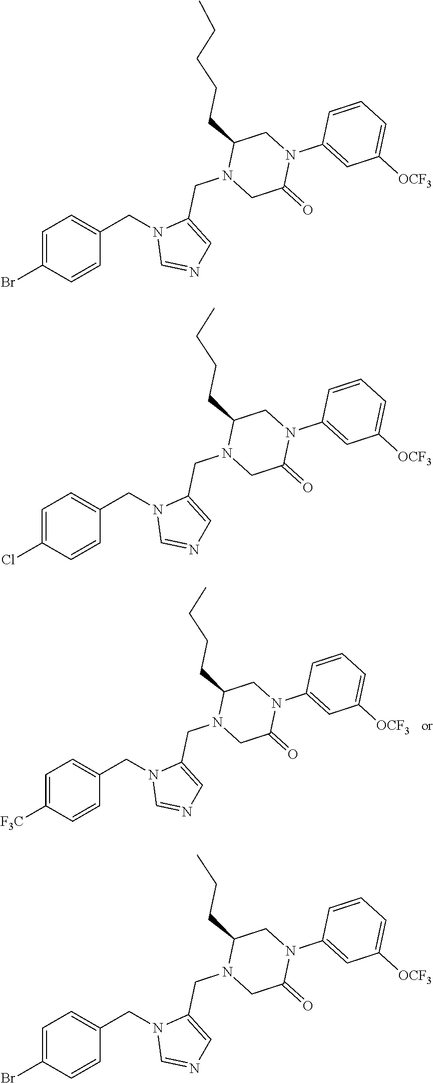
or a pharmaceutically acceptable salt thereof.

13. The method of claim 1, wherein the fungus is Cryptococcus neoformans.

14. The method of claim 1, wherein the inhibitor of a fungal farnesyl transferase that is competitive with respect to farnesyl disphosphate substrate is administered to a mammal to treat a fungal infection in the mammal.

15. The method of claim 14, wherein the inhibitor of farnesyl transferase is compound 2f: 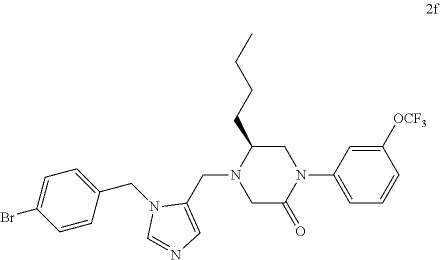 [SMILES: CCCC[C@H]1CN(c2cccc(OC(F)(F)F)c2)C(=O)CN1Cc1cncn1Cc1ccc(Br)cc1]
or a pharmaceutically acceptable salt thereof.

16. The method of claim 14, wherein the inhibitor of farnesyl transferase is: 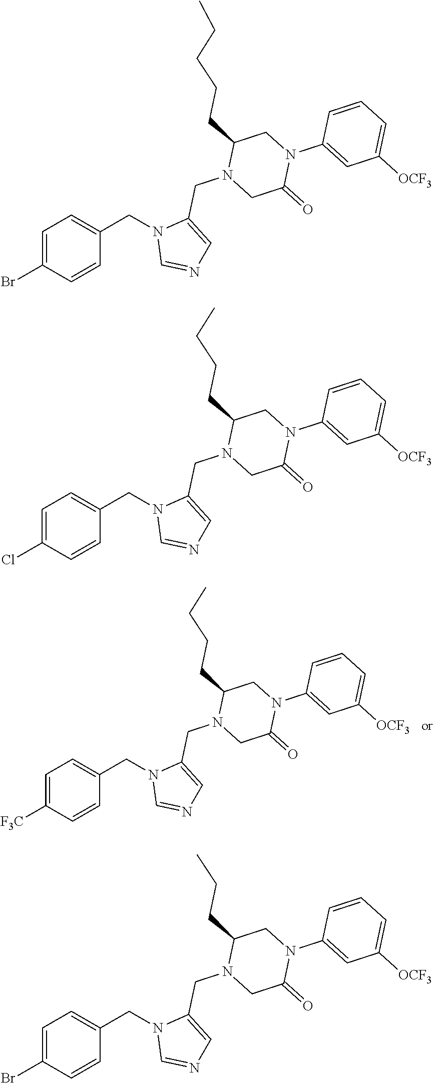
or a pharmaceutically acceptable salt thereof.

17. Compound 2f: 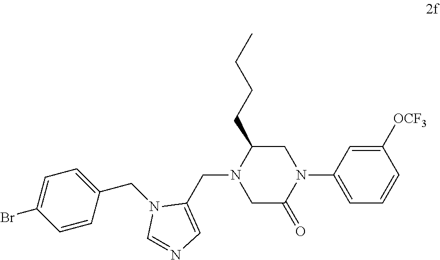 [SMILES: CCCC[C@H]1CN(c2cccc(OC(F)(F)F)c2)C(=O)CN1Cc1cncn1Cc1ccc(Br)cc1]
or a pharmaceutically acceptable salt thereof.

18. A pharmaceutical composition comprising Compound 2f as described in claim 17 or a pharmaceutically acceptable salt thereof and a pharmaceutically acceptable excipient.

19. The compound: 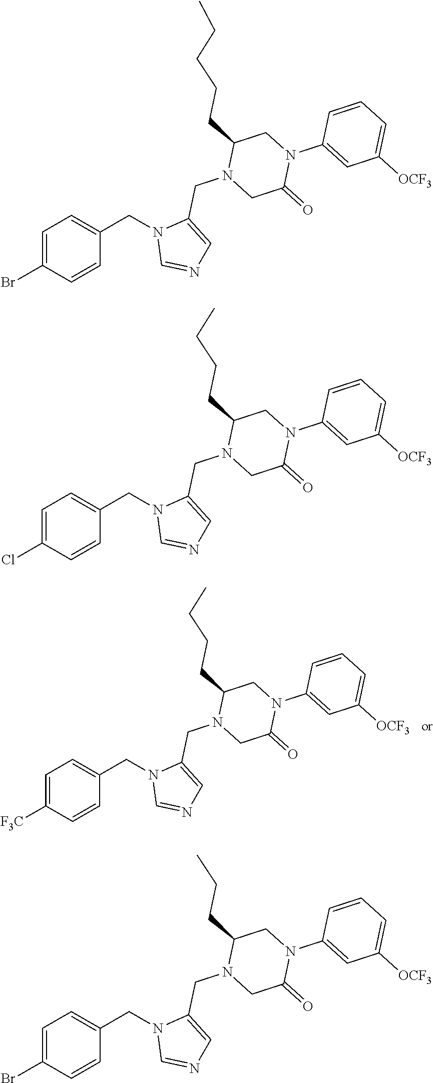
or a pharmaceutically acceptable salt thereof.

20. A pharmaceutical composition comprising a compound or pharmaceutically acceptable salt as described in claim 19 and a pharmaceutically acceptable excipient.
